(19) United States
(12) Patent Application Publication
(10) Pub. No.: US 2023/0044012 A1
(43) Pub. Date: Feb. 9, 2023

(54) DEVICE IMPLANTATION GUIDANCE

(71) Applicant: Navix International Limited, Road Town, Tortola (VG)

(72) Inventors: Shlomo BEN-HAIM, Milan (IT); Eli DICHTERMAN, Haifa (IL)

(73) Assignee: Navix International Limited, Road Town, Tortola (VG)

(21) Appl. No.: 17/789,199

(22) PCT Filed: Dec. 28, 2020
(86) PCT No.: PCT/IB2020/062487

§ 371 Date: Jun. 26, 2022

Related U.S. Application Data
(60) Provisional application No. 63/059,203, filed on Jul. 31, 2020.
(60) Provisional application No. 63/043,156, filed on Jun. 24, 2020.
(60) Provisional application No. 62/990,004, filed on Mar. 16, 2020.
(60) Provisional application No. 62/960,023, filed on Jan. 12, 2020.
(60) Provisional application No. 62/956,249, filed on Jan. 1, 2020.
(60) Provisional application No. 62/953,612, filed on Dec. 26, 2019.

(51) Int. Cl. A61B 34/20; A61B 17/12

(52) U.S. Cl. CPC A61B 34/20; A61B 17/12122; A61B 2034/2053; A61B 2017/1205; A61M 2025/0166

(57) ABSTRACT

Electrical field-guided positioning of a second device within a body cavity, using electrical field mapping information generated from electrical field measurements by electrodes of a first device. The first device, in some embodiments, is a catheter electrode probe, and the second device is an internally implantable and/or operated medical device. An exposed, electrically conductive portion of the second device is optionally configured to be used as an electrical field measuring electrode. A rule is applied to measurements made by this electrode to estimate its position within a body cavity. The rule is generated, in some embodiments, using measurements made by the first device. In some embodiments, electrical measurements are used to guide implantation verification. In some embodiments, electrical measurements are used to guide navigation at and through a septal wall between body cavities.

### RELATED APPLICATIONS

This application claims the benefit of priority of:

U.S. Provisional Patent Application No. 62/953,612, filed on Dec. 26, 2019;

U.S. Provisional Patent Application No. 62/956,249, filed on Jan. 1, 2020;

U.S. Provisional Patent Application No. 62/960,023, filed on Jan. 12, 2020;

U.S. Provisional Patent Application No. 62/990,004, filed on Mar. 16, 2020;

U.S. Provisional Patent Application No. 63/043,156, filed on Jun. 24, 2020; and

U.S. Provisional Patent Application No. 63/059,203, filed on Jul. 31, 2020; the contents of which are incorporated herein by reference in their entirety.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

### SUMMARY OF THE INVENTION

According to an aspect of some embodiments of the present disclosure, there is provided a method of guiding a transseptal crossing, including: accessing a 3-D structural model modeling at least a septum and a target region to be reached by a catheter after crossing the septum, and also modeling the septum and target region in their respective relative positions; accessing an estimation of an initial position of the catheter on a side of the septum opposite the target region, and accessing mechanical properties of the catheter; calculating a transseptal crossing location, based on the 3-D structural model, the target region, the estimation of the initial position, and the mechanical properties of the catheter, so that movements of the catheter from the estimated initial position of the catheter through the calculated transseptal crossing location are estimated to place the catheter on a trajectory to reach the target region; and presenting the calculated transseptal crossing position.

Also according to this aspect of some embodiments of the present disclosure, there is provided a system for guiding a transseptal crossing, the system including: a computer processor and memory containing instructions which instruct the computer processor to: access a 3-D structural model modeling at least a septum and a target region to be reached by a catheter after crossing the septum, and also modeling the septum and target region in their respective relative positions, access an estimation of an initial position of the catheter on a side of the septum opposite the target region, and accessing mechanical properties of the catheter, and calculate a transseptal crossing location, based on the 3-D structural model, the target region, the estimation of the initial position, and the mechanical properties of the catheter, so that movements of the catheter from the estimated initial position of the catheter through the calculated transseptal crossing location are estimated to place the catheter on a trajectory to reach the target region; and a display, wherein the processor is further instructed to control the display to present the calculated transseptal crossing position.

According to some embodiments of the present disclosure, the calculated transseptal crossing location is estimated to allow the movement of the catheter beyond the transseptal crossing location to the target region without adjusting a steering angulation of the catheter.

According to some embodiments of the present disclosure, the transseptal crossing location is a location of a fossa ovalis of an interatrial septum.

According to some embodiments of the present disclosure, the target region is a location within an ostium of a left atrial appendage.

According to some embodiments of the present disclosure, the mechanical properties of the catheter include a stiffness of the catheter.

According to some embodiments of the present disclosure, the calculating uses stiffness of the catheter adjusted for an initial steering angulation of the catheter.

According to some embodiments of the present disclosure, the calculating uses stiffness of the catheter adjusted for bending of the catheter.

According to some embodiments of the present disclosure, the 3-D structural model is generated using structural data obtained by measurements made using an electrode of an intracardiac probe.

According to some embodiments of the present disclosure, the estimation of the initial position of the catheter includes measured positions of the catheter.

According to some embodiments of the present disclosure, the estimation of the initial position of the catheter includes a simulated position of the catheter.

According to some embodiments of the present disclosure, the method further includes presenting adjustments to the catheter estimated to cause the catheter to assume the simulated position.

According to an aspect of some embodiments of the present disclosure, there is provided a method of guiding transseptal advance of a catheter, the method including: accessing a 3-D structural model modeling at least a septum and a target region to be reached by the catheter after crossing the septum, and also modeling the septum and target region in their respective relative positions; accessing an estimated initial position of the catheter on a side of the septum opposite the target region, and accessing mechanical properties of the catheter; calculating a transseptal trajectory of the catheter representing its advance from the estimated initial position across the septum, based on the 3-D structural model, the estimated initial position, and the mechanical properties of the catheter;

and presenting the calculated transseptal trajectory.

Also according to this aspect of some embodiments of the present disclosure, there is provided a system for guiding transseptal advance of a catheter, the system including: a computer processor and memory containing instructions which instruct the computer processor to: access a 3-D structural model modeling at least a septum and a target region to be reached by the catheter after crossing the septum, and also modeling the septum and target region in their respective relative positions, access an estimated initial position of the catheter on a side of the septum opposite the target region, and accessing mechanical properties of the catheter, and calculate a transseptal trajectory of the catheter representing its advance from the estimated initial position across the septum, based on the 3-D structural model, the estimated initial position, and the mechanical properties of the catheter; and a display, wherein the processor is further instructed to control the display to present the calculated transseptal trajectory.

According to some embodiments of the present disclosure, presenting the calculated transseptal trajectory includes presenting the 3-D structural model and the calculated transseptal trajectory registered to the model.

According to some embodiments of the present disclosure, the presenting includes presenting the target region along with the transseptal trajectory in a manner indicating how close the transseptal trajectory comes to the target region.

According to some embodiments of the present disclosure, the target region is a location within an ostium of a left atrial appendage.

According to some embodiments of the present disclosure, the estimated transseptal trajectory includes a trajectory of catheter advance beyond the septum without adjusting steering angulation of the catheter.

According to some embodiments of the present disclosure, the transseptal trajectory crosses the septum through a fossa ovalis of an interatrial septum.

According to some embodiments of the present disclosure, the mechanical properties of the catheter include a stiffness of the catheter.

According to some embodiments of the present disclosure, the calculating uses stiffness of the catheter adjusted for an initial steering angulation of the catheter.

According to some embodiments of the present disclosure, the calculating uses stiffness of the catheter adjusted for bending of the catheter.

According to some embodiments of the present disclosure, the 3-D structural model is generated using structural data obtained by measurements made using an electrode of an intracardiac probe.

According to some embodiments of the present disclosure, the 3-D structural model is generated using structural data obtained from a CT image.

According to some embodiments of the present disclosure, position cloud structural data obtained by measurements made using an electrode of an intracardiac probe are registered to the CT image, and the registration is used to estimate the position of the intracardiac probe.

According to some embodiments of the present disclosure, the estimated initial position includes measured positions of the catheter.

According to some embodiments of the present disclosure, the estimated initial position includes a simulated position of the catheter.

According to some embodiments of the present disclosure, the presenting includes presenting adjustments to the catheter to allow it to assume the simulated position.

According to an aspect of some embodiments of the present disclosure, there is provided a method of determining a degree of occlusion obtained by deployment of an occlusive device to divide a body lumen, the method including: accessing electrical impedance measurements measured using a portion of the occlusive device as an electrode during deployment of the device; estimating a degree of occlusion based on the measured electrical impedance; and presenting an indication of the estimated degree of occlusion.

According to some embodiments of the present disclosure, the estimating includes classifying, using a computer processor, the electrical impedance measurements to obtain an estimated degree of occlusion.

According to some embodiments of the present disclosure, the estimating includes analyzing a period of unstable electrical impedance measurements recorded after deployment.

According to some embodiments of the present disclosure, a period for assessing the electrical impedance measurement stability includes at least five seconds after an initial drop in electrical impedance during deployment of the occlusive device.

According to some embodiments of the present disclosure, the at least five seconds begin after a partial rebound in electrical impedance during deployment of the occlusive device.

According to some embodiments of the present disclosure, the classifying includes identifying an initial drop in impedance followed by a partial rebound in impedance as indicating deployment of the occlusive device within a confined location.

According to some embodiments of the present disclosure, the degree of occlusion is estimated based on a time course of the measured electrical impedance as the dielectric contrast agent redistributes.

According to some embodiments of the present disclosure, the occlusive device occludes an opening between a left atrial appendage and a remaining lumen of a heart left atrium.

According to an aspect of some embodiments of the present disclosure, there is provided a method of determining a degree of occlusion obtained by deployment of an occlusive device to divide a body lumen, the method including: injecting a dielectric contrast agent on at least one side of the occlusive device; measuring changes in the electrical properties of fluid on at least one side of the occlusive device as the dielectric contrast agent redistributes; and presenting an indication of the degree of occlusion, wherein the indication is based on the measured changes.

According to some embodiments of the present disclosure, the measuring is performed using an electrode within a compartment of the body lumen targeted to be closed off by the deploying.

According to some embodiments of the present disclosure, the electrode includes a portion of the occluding device.

According to some embodiments of the present disclosure, the injecting includes pressing the dielectric contrast agent across a membrane of the occlusive device into the chamber of the body lumen targeted to be closed off by the deploying.

According to some embodiments of the present disclosure, the method includes classifying the changes in the electrical properties, using a computer processor, to obtain an estimated degree of occlusion, and presenting the estimated degree of occlusion.

According to some embodiments of the present disclosure, the classifying includes identifying a timecourse with which the changes in electrical properties return to their values pre-injection.

According to some embodiments of the present disclosure, a timecourse including at least 40 seconds elapsed before returning to pre-injection values is classified as effective occlusion.

According to some embodiments of the present disclosure, the injecting includes injecting the dielectric contrast agent as a series of boluses, and wherein the changes measured are changes in the time course of measurements made after each bolus.

According to some embodiments of the present disclosure, the body lumen includes lumenal portions of a heart, and redistribution of the dielectric contrast agent is driven at least in part by beating of the heart.

According to some embodiments of the present disclosure, the occlusive device occludes an opening between a left atrial appendage and a remaining lumen of a heart left atrium.

According to an aspect of some embodiments of the present disclosure, there is provided a method of representing a deployment state of a medical device within a body, the method including: measuring, using the medical device as an electrode, an electrical parameter corresponding to a range of conformational states of the medical device; selecting a particular conformational state from the range of conformational states, based on the electrical parameter; and presenting an image indicating the selected conformational state.

According to some embodiments of the present disclosure, the measuring includes measuring an impedance, and the electrical parameter includes a decrease in impedance as the medical device expands.

According to some embodiments of the present disclosure, the measuring includes measuring a position of the medical device within one or more electrical fields, and the electrical parameter includes a decrease in impedance as the medical device expands.

According to some embodiments of the present disclosure, the presenting includes presenting a schematic representation of the medical device, wherein the schematic representation of the medical device indicates at least a current position of a distal tip of the device, and a width of the device.

According to some embodiments of the present disclosure, the presenting includes presenting a schematic representation of the medical device, wherein the schematic representation of the medical device indicates an estimated distal-most position of the medical device, once the medical device is fully deployed from a current position of the device by one or both of extruding the medical device from a device sheath, and retracting the device sheath from the medical device.

According to an aspect of some embodiments of the present disclosure, there is provided a method of controlling views shown during an intracardial catheterization procedure using a catheter, the method including: defining, for each of a first and a second display configuration, an updatable overview image of the catheter together with an updatable face-on view of a surface toward which the catheter travels; defining additionally, for the second display configuration, two updatable and mutually orthogonal lateral-side image views of the catheter; and showing and updating: the first display configuration during transseptal crossing of an interatrial septum, and then the second display configuration during navigation of the catheter to a position within the left atrium.

According to some embodiments of the present disclosure, a switch between the first and second display configurations is automatic, upon entering the left atrium with the catheter.

According to some embodiments of the present disclosure, a switch between the first and second display configurations is controlled by a user interface input.

According to some embodiments of the present disclosure, the image views of the second display configuration indicate the position of a device delivered by the catheter, in a current estimated position of the device.

According to some embodiments of the present disclosure, the lateral-side image views of the second display configuration display relative positions of the device and tissue of a body lumen around the device.

According to an aspect of some embodiments of the present disclosure, there is provided a method of displaying guidance for movement of a catheter within a body lumen, including: accessing a 3-D structural model of the body lumen; determining an entry region and a target region between which the catheter is to be navigated; and presenting an image including 3-D surfaces of the 3-D structural model for the entry region and the target region, in their modeled relative positions but disconnected from each other, along with a representation of the catheter occupying a region between the entry region 3-D surfaces and the target region 3-D surfaces.

Also according to this aspect of some embodiments of the present disclosure, there is provided a system for displaying guidance for movement of a catheter within a body lumen, the system including: a computer processor and memory containing instructions which instruct the computer processor to: access a 3-D structural model of the body lumen, and determine an entry region and a target region between which the catheter is to be navigated; and a display, wherein the processor is further instructed to control the display to present an image including 3-D surfaces of the 3-D structural model for the entry region and the target region, in their modeled relative positions but disconnected from each other, along with a representation of the catheter occupying a region between the entry region 3-D surfaces and the target region 3-D surfaces.

According to some embodiments of the present disclosure, at least 80% of an interior surface of the body lumen is not represented by the image.

According to an aspect of some embodiments of the present disclosure, there is provided a method of locating a fossa from an entry location of a catheter into a right atrium of a heart, the method including: determining a medial direction from the entry location, locating a wall of the right atrium in the medial direction; locating a region most distal from the entry location along the located wall; and presenting the located region with an indication that the location comprise a fossa of an interatrial septal wall of the heart.

Also according to this aspect of some embodiments of the present disclosure, there is provided a system for locating a fossa from an entry location of a catheter into a right atrium of a heart, the system including: a computer processor and memory containing instructions which instruct the computer processor to: determine a medial direction from the entry location, locate a wall of the right atrium in the medial direction, and locate a region most distal from the entry location along the located wall; and a display, wherein the processor is further instructed to control the display to present the located region with an indication that the location comprise a fossa of an interatrial septal wall of the heart.

According to an aspect of some embodiments of the present disclosure, there is provided a method of determining a degree of occlusion obtained by deployment of an occlusive device to divide a body lumen, the method including: accessing electrical impedance measurements measured using body surface electrodes; estimating a degree of occlusion based on the measured electrical impedance; and presenting an indication of the estimated degree of occlusion.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A is a flowchart schematically illustrating a method of navigating a device within a body cavity using electrical field measurements, according to some embodiments of the present disclosure;

FIGS. 1B-1E schematically illustrate an example of the method of FIG. 1A applied to device positioning (of a first device, and then a second device) in a body cavity (in the illustrated example, a left atrium—shown in cross-section—of a heart), according to some embodiments of the present disclosure.

FIG. 1F schematically illustrates shows an overview of left atrium in relation to a heart, according to some embodiments of the present disclosure;

FIG. 2 is a flowchart schematically illustrating a method of positioning a second device using a combination of a mimicking configuration measurements and changed-shape configuration measurements, according to some embodiments of the present disclosure;

FIG. 3 is a flowchart schematically illustrating a method of calibrating electrical field measurements made using a second device within a body cavity to electrical field measurements made using a first device within the body cavity, according to some embodiments of the present disclosure;

FIGS. 4A-4C schematically illustrate movement of an equivalent first-device electrode position of a cage-shaped second device as the second device deploys, according to some embodiments of the present disclosure;

FIGS. 4D-4G schematically illustrate conformational changes of a cage-shaped second device as the second device deploys, according to some embodiments of the present disclosure; FIGS. 4H-4J schematically represent sheath-withdrawal (FIG. 4H) and device-extruding (FIG. 4I) deployment of an LAAO device to the deployed state of FIG. 4J, according to some embodiments of the present disclosure;

FIGS. 5A-5C schematically illustrate movement of an equivalent first-device electrode position of an “umbrella”-shaped second device as the second device deploys, according to some embodiments of the present disclosure;

FIGS. 6A-6C schematically illustrate movement of an equivalent first-device electrode position of a bent-linear second device as the second device deploys, according to some embodiments of the present disclosure;

FIG. 7 schematically illustrates a system for navigating a device within a body cavity using electrical field measurements, according to some embodiments of the present disclosure;

FIG. 8 schematically represents an implantable device for modifying the circumference of a heart valve, and comprising a plurality of electrically conductive fasteners used to secure implantable device to the wall of a heart left atrium, according to some embodiments of the present disclosure;

FIG. 9A is a flowchart schematically representing a method of selecting a location for transseptal crossing by a device delivery sheath, according to some embodiments of the present disclosure;

FIGS. 9B-9C schematically illustrate configurations of elements involved in the method of FIG. 9A, according to some embodiments of the present disclosure;

FIG. 9D is a flowchart schematically representing a method of calculating a ballistic trajectory of a catheter sheath, according to some embodiments of the present disclosure;

FIG. 9E is a flowchart schematically representing a method of estimating transseptal crossing positions posing an elevated risk of aortic penetration, according to some embodiments of the present disclosure;

FIG. 10A is a flowchart schematically representing a method of verifying implant positioning, according to some embodiments of the present disclosure;

FIGS. 10B-10C are schematic graphs of impedance over time for stable and unstable closure by an LAAO device, according to some embodiments of the present disclosure;

FIG. 10D schematically represents an electrical measurement configuration related to the measurements of FIGS. 10B-10C, according to some embodiments of the present disclosure;

FIG. 11A is a flowchart schematically representing a method of verifying surgical closure of an opening between two fluid-filled compartments of a body lumen, according to some embodiments of the present disclosure;

FIG. 11B shows example time courses of the movement of an injected iodine solution across leaking and closed deployments of an LAAO device, according to some embodiments of the present disclosure;

FIG. 11C shows example time courses of the movement of an injected saline solution across leaking and closed deployments of an LAAO device, according to some embodiments of the present disclosure;

FIG. 11D illustrates closed vs. leaking timecourse measurements for a population of LAAO closure trials, according to some embodiments of the present disclosure;

FIG. 12A is a schematic flowchart of a method of showing a device deployment state based on measurements of its position, and parametric measurement of the device's degree of deployment, according to some embodiments of the present disclosure;

FIG. 12B illustrates a presentation of an estimated state of deployment of a LAAO device, according to some embodiments of the present disclosure;

FIG. 12C illustrates a schematic LAAO device representation from an oblique viewing angle, according to some embodiments of the present disclosure;

FIG. 12D schematically represents data sources which optionally provide parametric indications of degree of device deployment, and aspects of the state of deployment determined from the parametric indications according to some embodiments of the present disclosure;

FIGS. 13A-13B represent four-angle views of a-D scene presenting positioning (FIG. 13A) and deployment (FIG. 13B) of an LAAO device, according to some embodiments of the present disclosure;

FIG. 13C represents a two-angle view of a-D scene related to guidance of septal wall penetration, according to some embodiments of the present disclosure;

FIG. 14 is an overview flowchart of a method of deploying an LAAO (left atrial appendage occlusion) device, according to some embodiments of the present disclosure;

FIG. 15A schematically represents a cloud of position measurements made within a right atrium, from which the position of a fossa is estimated, according to some embodiments of the present disclosure.

FIGS. 15B-15C schematically represents a reconstructed-D view of a right atrium externally (FIG. 15B) and of a cutaway view of a right atrium looking toward the interatrial septal wall (FIG. 15C), according to some embodiments of the present disclosure;

FIG. 16A schematically represents a method of sizing an LAA, according to some embodiments of the present disclosure;

FIG. 16B schematically represents relative rates of dilution (on the vertical axis) of an impedance and/or dielectric contrast agent as a function of measurement position, according to some embodiments of the present disclosure;

FIG. 16C represents a cutaway view of a portion of the internal lumenal surface of a left atrium, according to some embodiments of the present disclosure;

FIG. 17A illustrates a segmented CT image of a portion of a heart, according to some embodiments of the present disclosure;

FIGS. 17B-17C illustrate registration of electrically-measured probe positions represented as a point cloud to a segmented CT image of a portion of a heart according to some embodiments of the present disclosure;

FIG. 18 is an overview flowchart of a method of deploying an LAAO (left atrial appendage occlusion) device, according to some embodiments of the present disclosure.

FIG. 19 schematically depicts a dilator inserted into a sheath assembly, according to some embodiments of the present disclosure;

FIGS. 20A-20B schematically depict views of a distal portion of a sheath assembly, according to some embodiments of the present disclosure;

FIGS. 21A-21B schematically depict views of a dilator distal portion of a dilator, according to some embodiments of the present disclosure; and

FIG. 22 schematically depicts a pigtail catheter, according to some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

An aspect of some embodiments of the present disclosure relates to the electrical field-guided positioning of a second device within a body cavity, using electrical field mapping information generated from electrical field measurements by electrodes of a first device. Herein, electrical field measurements may also be referred as “electrical measurements” or “electrical signal(s)”. Electrical field measurements optionally include, for example, measurements of voltage, current, impedance and/or another dielectric property. A typical parameter of electrical field measurement is impedance, which may itself be determined, e.g., from measurements of voltage made within one or more time-varying electrical fields.

Herein, the term “first device” refers to an electrode-carrying probe (a “probe”)—for example, an electrode catheter—insertable to and navigable within a body cavity; and configured to make electrical field measurements within the body cavity. In some embodiments, the first device carries a plurality of electrodes.

Herein, the term “second device” refers to medical devices, such as medical implements insertable to and navigable within a body cavity (e.g., to move the implement to a site of implantation and/or endolumenal operation). The medical implement (i.e., second device) may be any device insertable through a catheter, and/or another instrument such as an endoscope. Examples of medical implements (i.e., second devices) include: implantable pacemaker, stent, implantable ring, implantable valve replacement (e.g., aortic valve replacement, mitral valve replacement and/or tricuspid valve replacement), left atrial appendage (LAA) occluder, and/or atrial septal defect (ASD) occluder.

In some embodiments, electrical field mapping information comprises a map which associates measurements made by the electrodes of the first device to positions of those measurements (that is, the measurement data are position-mapped). Using electrical field mapping to guide movements of devices within a body cavity provides potential advantages, for example by reducing or eliminating a need for use of imaging techniques which use ionizing radiation (e.g., isotope and/or X-ray imaging) and/or access-limited imaging resources (e.g., MRI).

In some embodiments, using the electrical field mapping comprises application of a rule based on the map. The rule, in some embodiments, comprises direct lookup within the map, as if the electrical field measurement made by the second device was a measurement made by the first device. In some embodiments, the rule comprises transformation of the lookup result and/or the map to match second-device measurements to the map.

A rule optionally comprises other aspects. For example, it may comprise one or more of:
Steps of selection from among different maps and/or map transformations to suit particular conditions of the second device (e.g., different configurations, conformations, and/or degrees of unsheathing).
Particular conditions of electrical field generation (e.g., which fields are being generated at any given moment of a procedure).
Particular regions of navigation (e.g., there may be different transformations of the field mapping information, each suitable for finding the position of the second device in some region of the body cavity, but not necessarily suitable in another region; that is, a certain rule is not necessarily suitable for a complete and accurate transformation of all the field mapping information).

In some embodiments, the rule is defined so that the electrical fields measured using the second device are a subset of electrical fields used in measurements made by the first device (e.g., three or more electrical fields are measured during first-device mapping, but only two or three electrical fields are measured for positioning of the second device).

Herein an electrical field generated at two different times using substantially the same generating device parameters, e.g., of voltage, current, frequency, and generating electrode configuration, is considered to be practically the same electrical field. This is regardless of minor actual variations in the electrical field due to factors such as body movement and/or electrical component property drift, although these may be considered as contributing time-variability (or “noise”) to the electrical field. In some embodiments, the electrical field is continuously generated from electrodes which remain in place. However, these conditions are not strictly necessary to recreate the same electrical field parameters, and accordingly the same electrical field. For example, an electrical field generator can be briefly turned off and then on again, and the electrical field generated is still “the same” electrical field, as the concept of electrical field identity is used herein.

Furthermore, the definition herein of the identity of an electrical field over time discounts influences of measurement devices on the electrical field. To the extent that such influences occur (e.g., because the measuring device's impedance is not infinite), they are considered to modify an electrical field that still retains its “identity”.

In some embodiments, the first device comprises a sensing catheter or other device comprising 2, 3, 4, or more electrodes, and the electrical field mapping information is derived in part by using one or more known distances between the electrodes (inter-electrode distances) to set distance scaling of simultaneous electrical field measurements from each other. This scaling provides a constraint that allows estimation of relative position upon application of the constraint to a set having a suitably large number of measurements made as the first device moves about within the body cavity. The first device may itself be navigated and/or have its position determined using this estimation of relative position.

Construction and first-device use of maps of this type may be performed, for example, as described in International Patent Publication No. WO2018/130974 and/or in International Patent Publication No. WO2019/034944, the contents of which are included herein by reference in their entirety. Herein, measurement data that are position-mapped using the known inter-electrode distances are said to be “self-scaled”.

The inventors have realized that the self-scaled position-mapped measurement data (for example) provides a basis for navigation of a second device, even if the second device has only one electrode. Accordingly, in some embodiments, the method provides for navigation based on single-electrode measurements of a second device, based on self-scaled, position-mapped measurements made by a plurality of measuring electrodes of a first device—even though the second device is unsuitable for self-scaled position mapping.

This represents a potential advance, insofar as electrical field-based measurements by a single-electrode device as such are generally not susceptible to use with self-scaled position-mapped measurements (since there is only one electrode, there is no well-known inter-electrode distance).

While methods of electrical field-based guidance which do not use self-scaling are also known, their use is not always available and/or suitable. For example, these may rely on calculations of field geometry and/or careful configuration of how the electrical fields are generated, which are potentially unavailable, time-consuming, and/or prone to errors in setup and/or model assumptions.

Nevertheless, it should be understood that the electrical field mapping information used in some embodiments of the present disclosure to estimate second-device positioning is optionally obtained by a method that does not rely on self-scaling; for example, systems wherein electrical fields are generated to control their voltage gradient geometry (e.g., to be substantially linear within a certain region of interest), and/or systems wherein electrical field voltage gradients are calculable from other available information such as electrode positions and the dielectric properties and structure of body tissue through which electrical fields are generated.

Furthermore, the inventors have realized that when electrodes of both the first device and the second device are of sufficiently similar size and also of sufficiently similar impedance (e.g., pass minimal currents), any differences in how each “sees” the electrical field is potentially minimal enough that relative measurement differences made using what are essentially two different measurement systems can be ignored.

In some embodiments, an actual difference in one or more of these conditions (or another condition affecting measurement similarity) is compensated for by calibration, and/or accounted for as a limitation on the accuracy and/or precision of position estimation. For example, differences can optionally be reduced by the application of corrections which can be learned from the measurements themselves, and/or by calibration steps using equipment available at the time of the procedure, and/or pre-programmed for known devices.

For example, systematic offsets and/or scale differences in the measurements can be corrected by checking for measurement differences at positions which are well-defined outside of the electrical field measurements themselves. As examples, measurements from two different devices are estimated, in some embodiments, to originate from comparable positions if they are both made by one or more of:
At a same point of insertion (such as a heart interatrial septum).
While both the first and second device occupy adjacent positions, e.g., in contact with each other or at a known offset from each other.
Along a same path (for example along a same arc of a catheter as it advances into the body cavity).
At same extremes of available movement limited by lumenal walls of the body cavity.
At same landmarks such as blood vessel branching points, blood vessel ostia, heart valves, tissue folds, and/or appendages (such as the left atrial appendage).
At same electrically measured landmarks, such as local or global minima and/or maxima of measured voltage, or maximum- or minimum-amplitude slope (local or global) of voltage change; including features which include joint consideration of a plurality of voltage fields generated with different orientations, frequencies, and/or currents.
At any other “same position”, however this may be identified.

Potential advantages in using electrical measurements from a second device in order to guide its navigation and/or estimate its position include reducing or eliminating a need for imaging and/or position finding using exogenous measurement methods. For example, it may be possible to eliminate use of an esophagus-inserted (or other endoscopically positioned) ultrasound transducer. This may in turn obviate a need for the use of general anesthesia and/or the need for an attending anesthesiologist. Patient recovery time may also be faster for procedures performed without the use of general anesthesia. These effects also potentially decrease overall costs associated with a procedure.

An aspect of some embodiments of the present disclosure relates to the electrical field-guided positioning of a second device within a body cavity, using electrical field mapping information generated from electrical field measurements by electrodes of a first device, wherein the second device is quite different in shape and/or electrical characteristics from electrodes of the first device. For example, the electrodes of the first device may be relatively small—e.g., a millimeter or two in length and/or diameter.

The second device, in contrast, may be an implantable device or other medical implement comprising an electrically conductive portion (used as the electrode portion of the device) of several millimeters, (e.g., least 2×, 3×, or 4× as large as the measurement electrodes of the first device, and/or of at least 5 mm, at least 10 mm, 20 mm, or more) in one or more dimensions, having a surface of such dimensions which is exposed and/or configured to be exposed upon intralumenal deployment and/or operation of the second device. That conductive portion is wired by an insulated wire, in some embodiments, to form the electrode (electrode portion) used in making electrical field measurements.

The conductive portion may be secondarily an electrode, having a form provided primarily for a non-measurement function: for example, as a support, stent, plug, screw, needle (e.g., a transseptal needle), net, valve, frame, and/or other device function. In some embodiments, the conductive portion deploys through a shape alteration, e.g., from a compact form (e.g., such as may be deliverable via a catheter) to an expanded form taken upon a deployment of the device. In some embodiments, the exposed surface of the conductive portion is exposed by preparing the device with removal of an insulating and/or partially insulating surface. For example, a coated-metal stent may have a portion of the coating removed in order to form an electrode.

In some embodiments, guidance of movement of the second device is performed using a rule which treats measurements made actually using the second device as if made by an electrode of the first device placed in some particular spatial relationship with the second device, for example, in some particular position and/or orientation in relation to the second device. Herein, this is referred to as the equivalent first-device electrode position. For example, if the electrode portion of the second device is a linear conductor extending in a substantially linear (constant voltage gradient) electrical field, then the equivalent first-device electrode position is optionally at the geometrical middle of the second device. Optionally, the “first-device” may be considered as being sufficiently small that changing integration effects as a function of electrode geometry do not cause a measurable change in apparent position. In the limit, a notional “first device” electrode is optionally treated as a point electrode, that is, as if it measured from a point in space—although an actual electrode has spatial extent.

Optionally, the equivalent first-device electrode position is fixed relative to the second device. In some embodiments, the equivalent first-device electrode position relative to the second device is dynamic. For example, insofar as the voltage gradients are known (from the map made using the first device), and the shape of the second device's conductive electrode portion is also known (e.g., based on its design and deployment stage), it can be determined what measurement would be made as the second device moves to different positions, and, accordingly, what the equivalent first-device electrode position is for each of those positions, even if not always the same relative to the position of the second device.

In some embodiments, calibrations (e.g., using any of the reference position types described herein above) take into account relative differences between first device and second device geometry. For example, an expanded second device potentially encounters a wall physically, even though its equivalent first-device position remains remote from the wall. The distance being known or estimated, a contact with the wall provides a potential calibration point for aligning, scaling, and/or confirming the alignment/scaling of the original first-device measurement map with measurements now made by the second device.

An aspect of some embodiments of the present disclosure relates to the electrical field-guided positioning of a second device within a body cavity, using electrical field mapping information generated from electrical field measurements by electrodes of a first device, wherein:
The second device is susceptible to assume a plurality of conditions which cause it to act as an electrode with different properties in the different conditions, and
Among the plurality of conditions is a subset of conditions wherein it most closely approximates electrical field measurement characteristics of one or more electrodes of the first device.

For example, in some embodiments, the electrodes of the first device may be relatively small—e.g., a millimeter or two in length and/or diameter.

The second device, in contrast, may be an implantable device comprising an electrically conductive portion of several millimeters in one or more dimensions (e.g., in a range of 5-30 mm). That conductive portion is wired, in some embodiments, to form the electrode used in making electrical field measurements.

In some embodiments, the second device is delivered through and/or within an electrically insulating sheath. As the device leaves the sheath, in some embodiments, it gradually assumes different electrical properties affecting how it “sees” the electrical fields around it. In some embodiments, navigation of the second device takes place while it is in a partially un-sheathed configuration which provides to it electrical field measurement characteristics similar to those of one or more of the electrodes of the first device. Herein, any such configuration is referred to as a “mimicking” configuration relative to one or more electrodes of the first device. For a second device in a mimicking configuration, the rule for applying electrical field mapping information obtained using electrodes of the first device is optionally as straightforward as a direct lookup. Optionally (for example if the mimicking configuration mimics imperfectly), scaling and/or offset corrections (if applied at all) are nevertheless made relatively simple and direct by use of the mimicking configuration. The inventors have found that a useful degree of mimicry is optionally obtained, in some embodiments, by extruding a second device until electrical field measurements begin to be received at measurement value levels corresponding (e.g., within about ±20%) to levels of first device electrode measurements at about the same location—and then stopping. Optionally, magnitudes of measurement noise are used as an indicator. For example, upon reaching, during device extrusion, an impedance low enough to make measurements with a root mean square noise amplitude similar (e.g., within ±20%) to the noise amplitude of first-device electrodes is used as a signal that a mimicking device configuration has been reached.

In some embodiments, the second device is reconfigurable during navigation between the mimicking configuration and another (for example, a more-unsheathed, more-extended and/or more-deployed) configuration. Optionally, this is used to obtain a direct calibration of the non-mimicking configuration to a rule using the electrical field mapping information from the first-device measurements. For example, the second device, in a mimicking configuration, makes measurements at a first position in the body cavity, and a rule applied to determine its position. It is then converted to a non-mimicking configuration, resulting in a known change in position relative to the determined position. More measurements are made. This can be repeated at one or more additional positions. The non-mimicking configuration, in some embodiments, comprises exposure of more of the surface of the second device outside an insulating sheath such as a catheter sheath; for example, at least 2×, 3×, 4×, or more surface along a longitudinal axis of the sheath than is exposed by an electrode of the first device. In some embodiments, the non-mimicking configuration comprises a conformational change in a shape of the second device, for example, a radial expansion of part of the device (e.g., an expansion to either side of a longitudinal axis of the device). Optionally, the radial expansion increases a diameter of the device by at least 2×, 3×, 4×, or more. In some embodiments, the non-mimicking configuration comprises exposing a surface of the second device which is specially formed to perform a non-electrical function. For example, the second device optionally comprises a screw thread, serrations, tool-receiving surface (e.g., a hexagonal recess), or another surface which is shaped to perform a mechanical function such as fastening, cutting, and/or engaging with a mating surface.

In some embodiments, mimicking/non-mimicking configuration pairs of measurements provide calibration indications which allow the first-device mapping data to be recalibrated to measurements by the second device in the non-mimicking configuration. In some embodiments, these measurements, optionally with suitable extrapolations and interpolations, are used stand-alone as a new set of position-mapped measurements, which indicate and/or guide further positioning of the second device.

In some embodiments, the second device is optionally itself used to make measurements of the types described in relation to the first device, e.g., a cloud of measurements which is used to generate a rule converts electrical field measurements into positions. In embodiments wherein the second device is not addressable as a plurality of (electrically mutually isolated) electrodes, this potentially limits methods by which the rule is generated, for example to a non-self-scaled method, optionally based on methods, principles, and/or systems for position estimation without self-scaling mentioned hereinabove.

A broad aspect of some embodiments of the present disclosure relates to the use of dielectric property measurements of body tissue to guide implantation of an implantable medical device. The measurement of dielectric properties, in some embodiments, is used to identify body structures within the vicinity of implantation. The measurement of dielectric properties, in some embodiments, is used to validate implantation. The measurement of dielectric properties, in some embodiments, is used to determine device position. Measurement of the dielectric property can be performed using the implanted device itself, an intrabody probe, and/or body surface electrodes.

A broad aspect of some embodiments of the present disclosure relates to systems and methods for guiding implantation of a left atrial appendage occlusion (LAAO) device. In some embodiments, implantation of an LAAO device comprises one or more of the operations of: reaching the right atrium through the superior and/or inferior vena cava, imaging a portion of the right atrium including the fossa ovalis, performing a transseptal crossing (e.g., through the fossa ovalis) by puncturing the interatrial septum, imaging the left atrial appendage (LAA), sizing the LAA in order to assist planning of selection and/or placement of an LAAO device, deployment of the LAAO device in the LAA, and verifying sealing of the LAA after deployment of the LAAO device. These operations are optionally performed using one or more techniques for electrical measurement-based guidance, including electrical measurement based imaging, electrical measurement based position finding, and/or other electrical measurement based techniques, for example as described herein. Potentially, using electrical measurements performed during a procedure reduces or removes a necessity for pre-planning, or allows corrections for pre-planning of the procedure in aspects such as selection of an LAAO device size and/or selection of a predetermined crossing location across the interatrial septum. Optionally, the overall LAA implantation procedure is performed without use of either trans-esophageal echocardiography or X-ray imaging. It should be understood that any of the electrical measurement-based techniques described herein is optionally performed in isolation from any of the other electrical measurement-based techniques. Measurements for electrical measurement-based operations are optionally performed using electrophysiology catheters comprising one or more measurement electrodes—on the catheter sheath itself, and/or one or more measurement electrodes on a tool introduced over the catheter; for example, a device delivery sheath, an electrophysiology probe as such (straight or loop shaped, and optionally with multiple electrodes, for example), a dilator, and/or a “pigtail” device. Optionally, a catheter guidewire is equipped with one or more electrodes used to perform electrical measurements.

An aspect of some embodiments of the present disclosure relates to methods of guiding the choice of a transseptal crossing location for catheters used during a procedure performed by minimally invasive techniques. More particularly, in some embodiments, the aspect relates to guiding choice of a transseptal crossing location in order to assist in reaching the left atrial appendage with a delivery sheath (catheter) of a left atrial appendage occlusion (LAAO) device. The advance is optionally simple (that is, by pushing a catheter, guidewire, and/or dilator forward without use of other control mechanisms) or with use of actuation of a control mechanism to steer the advancing device.

In some embodiments, the transseptal crossing location is calculated using a 3-D structural model of the anatomy modeling the septum, including the fossa ovalis, and a target region to be reached by the catheter after crossing the septum (e.g., an ostial position of the LAA). In some embodiments, the 3-D structural model comprises a V2R model which models a structure in a spatial domain R, based on reconstruction from electrical field measurements obtained in an electrical field-measurement domain V (e.g., voltage, impedance, or another electrical field measurement). In some embodiments, the 3-D structural model is generated using measurements made using one or more data sources. In some embodiments, the measurements together comprise a 3-D image such as may be obtained via CT scan, MRI scan, bi- or multi-planar X-ray imaging, and/or another external imaging method; and the structural model is or is derived from the 3-D image obtained. Additionally or alternatively, in some embodiments, the measurements are measurements made from an intracardiac probe, processed to map out intracardiac structures. The measurements may comprise, for example, electrical measurements (e.g., of impedance and/or an impedance-related property such as locally sensed voltages and/or currents), magnetic measurements, ultrasound measurements, or another modality of measurement. Herein the term “structural data” may refer to any such measurement, optionally limited to a mentioned measurement modality, e.g., “structural data obtained using electrical measurements”.

In some embodiments, more than one form of structural data is used to generate a 3-D structural model. The different forms of structural data are registered to each other, for example as described for 3-D images (e.g., CT and/or MRI images) and clouds of electrical readings (e.g., electrical measurements performed so as to provide position information) in International Patent Publication No. WO2018/078540, the contents of which are included by reference herein in their entirety.

In some embodiments, the transseptal crossing location is calculated using mechanical properties of the delivery sheath; for example, its stiffness, bending radius, and/or steering capabilities. Optionally, the mechanical properties of the delivery sheath are combined with mechanical interactions with the heart wall, e.g., it is taken into account how compliant the heart wall is compared to the delivery sheath when the two encounter one another.

In some embodiments, the transseptal crossing location is calculated using one or more estimated initial configurations of the delivery sheath. An estimated initial configuration may comprise a measured configuration (e.g., measured positions of the actual delivery sheath), and/or a simulated configuration. The simulated configuration, in some embodiments, is used as a reference to provide guidance to an operator indicating how the actual delivery sheath configuration may be adjusted so that assumes the estimated initial configuration. This guidance may comprise an image (e.g., which the operator adjusts an actual delivery sheath to match) and/or instructions (e.g., which the operator follows in order to adjust an actual delivery sheath).

In some embodiments, a motion which would result from advancing a delivery sheath from an estimated (current and/or simulated) position of the delivery sheath is calculated. In some embodiments, the motion comprises advancing catheter from the transseptal crossing location to the target region without changing steering actuation of the catheter.

Presentation to an operator, in some embodiments, comprises showing a position and/or configuration of the delivery sheath with respect to the structural model of the heart. The position presentation comprises one or more of, for example:
Showing an estimated configuration of the delivery sheath pre-crossing; optionally measured and/or simulated. The simulated configuration is optionally also a targeted pre-crossing configuration.
Showing an estimated and/or targeted crossing location of the delivery sheath through the septal wall.
Showing an estimated and/or targeted trajectory of the delivery sheath after is crosses the septal wall.

An aspect of some embodiments of the present disclosure relates to methods of determining a degree of occlusion obtained by deployment of an occlusive device to divide a body lumen. In some embodiments, the occlusion comprises dividing the body lumen into separate compartments on either side of the occlusive device. In some embodiments, the occlusion comprises closure of a portion of the body lumen into a separate compartment. In some embodiments, the occlusive device is a LAAO device, deployed within a LAA, to convert the LAA from a portion of the left atrium into a separate, closed compartment. In some embodiments, the occlusive device is at least partially porous, at least during a stage of implantation and/or delivery.

In some embodiments, the degree of occlusion is determined based on impedance measurements made using the occlusive device as an electrode. Particular features of impedance measurements, in some embodiments, include an initial drop of impedance during device expansion, a rebound of impedance as the device approaches lumenal walls of the body lumen being divided, and/or a period of relative stability or instability following completion of device expansion (e.g., “instability” is identified when the impedance measurements are unstable with a noise level at least 2×, 4×, 5× or 10× an inherent measurement noise of the system). The period of relative stability, in some embodiments, comprises at least five seconds of stable measurements.

It is noted that a potential advantage of using the occlusive device itself as a measurement device is that the occlusive device is configured, by its nature, to be left in place once a sufficient degree of occlusion is confirmed. There is, accordingly, no requirement to introduce another measurement device to confirm a degree of occlusion, which could either mean leaving an extra device behind, or somehow finding a way to extract a measurement device from a compartment which has already been occluded, potentially disrupting the occlusion in the process. Additionally or alternatively, in some embodiments, occlusion is indicated by impedance changes measured from another device, for example, impedance between body surface electrodes, between which there is an electrical pathway including the body lumen targeted for occlusion.

In some embodiments, the degree of occlusion is determined based on measurements of dielectric properties of fluid in the vicinity of the occlusive device, into which has been injected a dielectric contrast agent comprising, for example: hypertonic saline, ice cold saline, hypotonic saline, warm saline, iodine-containing solution (e.g., Iodixanol), or another solution with dielectric properties distinct from the dielectric properties of the fluid (e.g., blood) that normally fills the compartments into which the body lumen is to be divided.

In some embodiments, the measurements comprise measurement of voltage, current, and/or impedance as a function of time, upon injection of the dielectric contrast agent.

An aspect of some embodiments of the present disclosure relates to methods of monitoring a deployment state of a medical device to be operated within a body, wherein operation of the medical device comprises the medical device undergoing a conformational change. In some embodiments, the medical device to be operated is a left atrial appendage occlusion (LAAO) device, and the conformational change comprises expanding the LAAO device to occlude an ostium of an LAA, and thereby also implant the device.

In some embodiments, the LAAO device is configured to act as an electrode, e.g., by wiring it to an electrical measurement device through a catheter used as a delivery sheath of the LAAO device. Upon expansion, impedance measured through the device will tend to drop (at least initially), e.g., as an electrically exposed surface area of the device increases. In some embodiments, this impedance measurement is used as a parametric indication of device deployment state. In some embodiments, an operator is presented with an image that visually displays what an LAAO device (or other medical device) is expected to look like, based on a currently measured impedance value from the medical device.

Moreover, in some embodiments, electrical field measurements made using the device will tend to integrate field potentials, potentially producing measurement results that correspond to sampling of the same electrical field(s) by a smaller electrode at some “equivalent position” with an offset relative to the LAAO device (e.g., typically an equivalent position somewhere inside the device). This equivalent position corresponds to the “equivalent first device electrode position”, described hereinabove, with the smaller device being the first device. The smaller electrode is optionally small enough that integration effects have a negligible effect on position determination; e.g., a still-smaller electrode would appear, on the basis of its measurements, to be located at the same location.

As the LAAO device undergoes conformational change, this offset changes. The changing offset in measurement values is followed, in some embodiments, and optionally correlated with a changing spatial offset, e.g., by treating the measurement results as equivalent to electrical measurements made while mapping a body lumen in which the medical device is being deployed. In some embodiments, the measurement offset and/or spatial offset are used as parametric indications of deployment state. These parametric indications are optionally used to select an image presented to the user, for example as just described for embodiments wherein impedance is used as a parametric indication of deployment state. Optionally, just one electrical field is used; for example, since the offset of the device may itself comprise translation along a single linear dimension. Optionally, a plurality of electrical fields are used, e.g., to allow specifying position changes in three-dimensional spatial coordinates.

An aspect of some embodiments of the present disclosure relates to a method of displaying the navigational status of a catheter device using selectable combinations of viewing angles, according to some embodiments of the present disclosure. In some embodiments, navigation of catheter to a target location (e.g., a location for deployment of an LAAO device or another implantable device) comprises navigation to and/or through a plurality of navigation targets.

Positions in the three dimensional space in which a catheter device navigates are potentially difficult to clearly discern from any single viewing angle. Furthermore, navigation to different navigation targets potentially benefits from differential presentation of image information which guides the navigation.

A 3-D overview image is useful for keeping a global perspective of a procedure; and particularly in the case of catheter procedures, where a catheter's previous bends and routings act to profoundly affect how it will behave as it continues to be advanced. However, a 3-D overview image may obscure details important for local navigation, e.g., by making them too small to easily discern, and/or presenting them at a suboptimal angle.

In some embodiments, an appearance of the 3-D overview image is simplified by reducing the illustrated 3-D surfaces of the body lumen in which the catheter is represented to surfaces of an entry region, and surfaces of a target region, with the two surface representations disconnected from each other, but otherwise in their normal respective positions. The catheter itself is shown within the 3-D overview image at one or more estimated and/or suggested positions. Suppressing surfaces between the entry region and target region (for example, at least 50%, 70%, 80%, or 90% of interior surfaces of the represented body lumen), potentially emphasizes the position and/or conformation of the catheter itself, by reducing cluttering of the view by overlying and/or background lumenal surfaces which are not themselves affecting catheter navigation. Supporting this view also allows operators to optionally map a smaller portion of the heart, potentially speeding up the procedure.

In some embodiments of the present disclosure, the 3-D overview image is supplemented by one or more additional images which assist detailed maneuvering of a catheter, particular as it approaches a target. There may be more than one type of target which a catheter navigates in the course of a procedure; for example, a fossa ovalis may be transiently targeted during transseptal crossing, while a later target of the procedure comprises an opening of a lumenal structure such as an appendage (e.g., the LAA), vein (e.g., a pulmonary vein), or valve interior (e.g., a mitral valve).

A supplemental image may show, for example a “face on” view of what is in front of the catheter, optionally including a representation of the catheter itself. For cases where the current target comprises a surface feature of a lumenal wall (e.g., a fossa ovalis), the two dimensions of the facial view may be enough for targeting. A two-view display is presented, in some embodiments, comprising the face-on view and the 3-D overview image.

Where the target is not a surface feature (that is, it “floats in space”), one or more additional views are provided, in some embodiments, to assist in targeting. Moreover, in some embodiments, a surrounding context of the device is important, so that it is a potential advantage to see, e.g., how a device contacts surrounding tissue. In some embodiments, a four-view display is presented, comprising orthogonal lateral-side views of the device (e.g., sagittal and transverse views), additional to the face-on and 3-D overview images. Thee lateral-side views provide the potential advantage of allowing the advance in depth of the catheter to be clearly seen, e.g., as movement across one direction of each image. Another potential advantage of the lateral-side view is to allow tissue contacts to be visualized with respect to four different quadrants of the device; e.g., top and bottom, left and right. This is particularly of potential value for the implantation of an LAAO device, or another device wherein circumferential contact with tissue is a part of the device's function.

It is, however, a potential distraction and/or cognitive load to an operator to show extra views—e.g., to show four views when only two are needed. Moreover, where fewer views are needed, more display space can be devoted to showing details useful for navigation. Showing an appropriate number of views for a certain procedure potentially serves as a cue to an operator (distinguishable even via peripheral vision) to help maintain their sense of the progress and phase of the procedure.

In some embodiments, software is configured to track a current phase of a procedure, and select an arrangement of views to show accordingly. In some embodiments, tracking comprises user indications such as foot pedal or other user interface commands; e.g., commands issued upon entry to the right atrium (in preparation for transseptal crossing), and again after crossing the interatrial septum (in preparation for positioning before deployment of the device). In some embodiments, the view shown is selected automatically.

Different automatically detectable cues can be used for this selection. In some embodiments, a distal tip of a catheter is tracked (e.g., by means of electrical field measurements). For example, upon entering a region previously mapped as “right atrium”, the transseptal-type two-view arrangement is shown; upon entering a region previously mapped as “left atrium”, the LAAO device positioning four-view arrangement is shown. Optionally, right-atrium entry is tracked by another method, for example “first entry into an open space”, or “first entry into a space where an electrocardiogram typical of the right atrium” is detected. Similarly, left-atrium entry is optionally tracked by another method, for example, a sudden change in electrical measurements made by an electrode as it pushes into and then suddenly through the fossa.

In some embodiments, one or more of the views is associated with an indicator of viewing angle, optionally comprising a small representation of a heart or portion thereof.

There are potential advantages in using electrical measurements (e.g., as described in relation to any of the foregoing aspects) to guide navigation, positioning, deployment, and/or validation of a device (for example, an LAAO device) delivered to a heart by catheter. The potential advantages include reducing or eliminating a need for imaging and/or position finding using exogenous measurement methods. For example, it may be possible to eliminate use of an esophagus-inserted (or other endoscopically positioned) ultrasound transducer. This may in turn obviate a need for the use of general anesthesia and/or the need for an attending anesthesiologist. Patient recovery time may also be faster for procedures performed without the use of general anesthesia. These effects also potentially decrease overall costs associated with a procedure, including costs associated with equipment (e.g., avoiding a need for procedure-dedicated imaging and/or anesthesia equipment), facility (e.g., avoiding the need for a shielded X-ray room), and/or personnel (e.g., reducing staff needed to prepare and/or monitor equipment and/or anesthesia).

An aspect of some embodiments of the present disclosure relates to designs of sheath, dilator, and/or pigtail catheter configured for use in minimally invasive intervention procedures, for example, in sealing the left atrial appendage (LAA).

In some embodiments, the procedure includes a step of puncturing the interatrial septum.

In some embodiments, a sheath, sheathing a dilator with a needle inside the dilator, is used to navigate the dilator and the needle to the interatrial septum. At the septum, the dilator may be extended out of the sheath to sense more closely (e.g., using electrodes with which the dilator is provided) the septum, so as to identify the proper place to puncture the septum for a trans septal. Alternatively or additionally, the sheath (and electrodes provided thereto) may be used for identifying the right spot for the trans-septal crossing.

Once the right spot is identified, a needle may be extended from inside the dilator to puncture the interatrial septum. Then, the dilator may be used to dilate (i.e., broaden) the aperture opened by the puncture. Optionally, after dilation with a first dilator, the first dilator is retracted and taken out of the patient's body; and a second dilator, larger than the first, is inserted in order to further dilate the trans-septal aperture.

In some embodiments, the procedure includes a step of evaluating the depth of the LAA, e.g., to assist in the selection of an occluding device of suitable size. In some embodiments, to evaluate the depth to which the occluding device should be matched, a pigtail catheter with irrigation holes at its curled end and electrodes spaced apart proximally to the curled end may be inserted to the appendage. Irrigating the appendage with a suitable dielectric contrast medium (e.g., saline) changes the impedance read by the electrodes located inside the LAA. Outside the LAA, irrigation fluid is quickly diluted and/or pumped away, so that it has no or minimal effect on sensing by electrodes which remain outside the LAA.

Irrigation may be performed after pushing the pigtail to touch the back wall of the LAA, allowing the known length of the pigtail to serve as a measurement reference. Impedance changes at electrodes placed at different distances along the pigtail, indicate which electrodes are inside the LAA and which outside. From the known relative positions of the electrodes, the depth of the appendage can then be estimated.

In some embodiments, after the puncture is properly dilated, the LAA occluding device (e.g., a Watchman™ by Boston Scientific) is navigated to the LAA. The LAA occluding device may cross, inside the sheath, the septum from the right atrium to the left. After passing, the occluding device may navigate to the LAA, using features of the sheath and/or the occluding device for position sensing and/or movement.

In some embodiments, after deployment of an LAA occluding device, the deployment quality may be assessed using the sheath.

In some embodiments, a fluid dielectric contrast agent that is more (or less) conductive than blood is pushed from the sheath through the occluding device into the LAA (e.g., via small holes in a fabric mesh covering a portion of the LAA). Impedance calculated from measurements at one or more of the sheath's electrodes and/or at the occluding device itself, can be monitored. The impedance first goes up (if the contrast agent is less conductive than blood) or down (if the contrast agent is more conductive than blood), and then returns to baseline values. The return is quicker, if the occlusion is bad enough to allow leakage, or slower, if the occlusion is good enough to prevent, or at least substantially reduce, leakage from the appendage out to the atrium. Examples of contrast agents include: saline solution (more conductive than blood), and iodixanol, commercially available from GE Healthcare as Visipaque 320 (less conductive than blood).

In some embodiments, for use in support of the trans-septal crossing, the sheath has distal, peripheral electrodes configured to measure impedance of internal tissue contacted by the distal end of the sheath; and a proximal electrode, configured to measure impedance inside the blood pool in the RA, as a reference to the tissue impedance.

In some embodiments, for use in support of LAA closure verification, the sheath may have irrigation holes that allow irrigating the LAA with the contrast agent and monitoring the impedance development over time after the irrigation. Alternatively, irrigation holes may be omitted, and the irrigation may be from the distal end of the sheath only.

Herein, a device and/or probe of which an electrode portion is used to obtain electrical measurements from within a body cavity is also referred to equivalently as an “electrode carrying” device and/or probe. Conversely, such an electrode portion is an “electrode of” the device and/or probe. This terminology applies both to a portion with a dedicated use as an electrode, and to a portion with another purpose (e.g., structural support) which is also configured for use as an electrode.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Methods of Second Device Navigation

Second Device Navigation with Selected Operations Illustrated

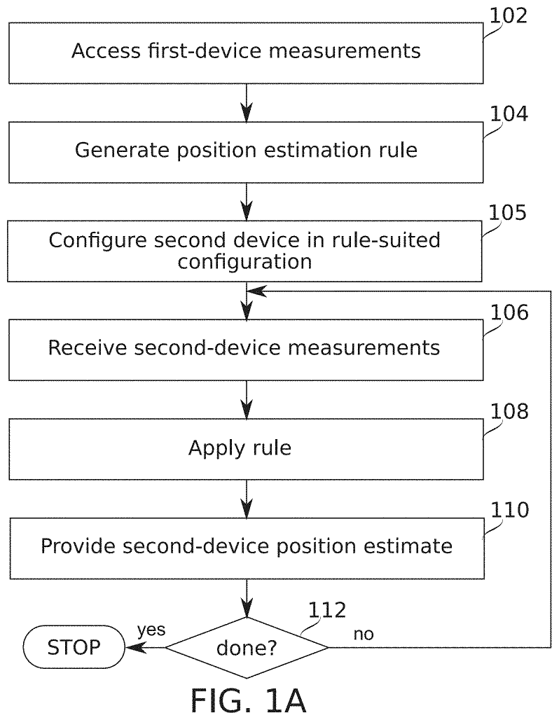

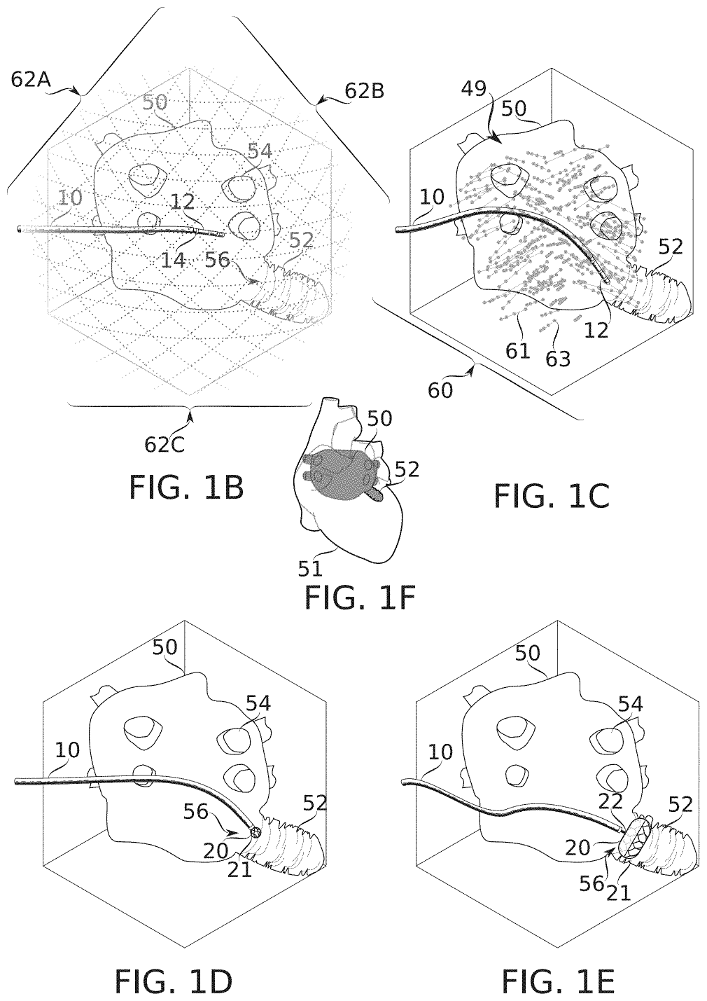

Reference is now made to FIG. 1A, which is a flowchart schematically illustrating a method of navigating a device within a body cavity 49 using electrical field measurements, according to some embodiments of the present disclosure. Further reference is made to FIGS. 1B-1E, which schematically illustrate an example of the method of FIG. 1A applied to device positioning (of a first device 12, and then a second device 20) in a body cavity 49 (in the illustrated example, a left atrium 50, shown in cross-section, of a heart 51), according to some embodiments of the present disclosure. Further reference is made to FIG. 1F, which schematically illustrates shows an overview of left atrium 50 in relation to a heart 51, according to some embodiments of the present disclosure.

The flowchart of FIG. 1A begins; and at block 102, in some embodiments, electrical field measurements are accessed which were obtained from a first device 12 comprising a plurality of electrodes configured to measure electrical fields generated to extend through the body cavity 49. In some embodiments, the measurements accessed are voltage measurements of electrical fields.

In some embodiments, first device 12 comprises an electrode catheter having two or more electrodes arranged at known distances from each other. A typical parameter of electrical field measurement is impedance, which may itself be determined, e.g., from measurements of voltage made within one or more time-varying electrical fields. First device 12 is illustrated in FIG. 1A as a straight-probe electrode catheter. However, it should be understood that an electrode catheter of another configurations is used in some embodiments, e.g., a lasso configuration (loop-ended probe).

In some embodiments, impedance is calculated from voltages and/or current measurements at electrodes of first device 12. In some embodiments, impedance is calculated from voltage measurements at electrodes of first device 12 and known current injected at electrodes of first device 12. In some embodiments, electrical measurement may include any dielectric parameter measured at one or more electrodes of first device 12. Electrical measurement may include voltage measurements, current measurements, impedance measurements and any combination thereof.

In some embodiments, the accessed measurements were obtained from a first device 12 when the first device 12 was used for mapping and/or reconstructing the body cavity; e.g., to obtain measurements for a map and/or reconstruction used in an interventional procedure. Mapping and/or reconstructing the body cavity may be performed, for example, as described in International Patent Publication No. WO2018/130974 and/or in International Patent Publication No. WO2019/034944, the contents of which are included herein by reference in their entirety. In some embodiments, measurements are accessed which were obtained from a first device 12 when the first device 12 is used solely for obtaining such measurements, e.g., for calculating field mapping information to be used in navigation of the second device.

FIGS. 1B-1C illustrate a first device 12 (introduced, for example, through catheter sheath 10) comprising a plurality of electrodes 14 and used to map a region of a left atrium 50 of a heart 51, including at least an ostial portion 56 of a left atrial appendage (LAA) 52, and/or one or more pulmonary vein ostia 54. The darkened region of FIG. 1F represents the location of a left atrium 50 and LAA 52 relative to heart 51.

At block 104, in some embodiments, a rule for transforming electrical field measurements to positions is generated, using the electrical field measurements made by the first device 12. Herein, such a rule is also referred to as a position estimation rule.

In some embodiments, the method begins with accessing (and not necessarily with the generating of) a position estimation rule. The position estimation rule may, for example, have been previously generated from measurements which were obtained from a first device 12 comprising a plurality of electrodes configured to measure electrical fields generated to extend through the body cavity 49.

Electrical fields 62A, 62B, 62C (represented as a crossing grid of dotted lines in FIG. 1B) represent three electrical fields generated to extend through a body cavity 49 (including, in the example, left atrium 50). In some embodiments, electrical fields 62A, 62B, 62C are generated to extend through a body cavity 49 by passing electrical currents between body surface and/or intrabody electrodes at different electrical potentials. In some embodiments, body surface electrodes are provided on body surface patches, e.g., 6 body surface patches. In some embodiments, an additional ground patch may be used, e.g., a ground patch positioned on the patient's right leg.

A plurality of electrical fields are generated (e.g., between different electrode sets, and optionally differentiated by being generated at different times and/or frequencies). Each electrical field may be treated as defining a different parametric coordinate of a coordinate system defined by the fields taken together. Measurement sets, each set measuring a plurality of electrical fields extending through an individual position within the body cavity 49, provide a distinctive set of co-ordinates for that position, and or a distinctive “tag”, insofar as each measurement set is distinct in its values from the other measurement sets.

Generating electrical fields having mutually orthogonal voltage gradient components helps ensure that each position is associated with a different combination of electrical field measurements. However, the electrical field gradients are not necessarily orthogonal. While three voltage fields are illustrated in FIG. 1B, this is for purposes of illustration, and the number of voltage fields used may be larger or smaller.

The marking dots 63 used to represent of measurement cloud 60 (FIG. 1C) represent different positions of first device 12 at which corresponding different sets of electrical field measurements are made. Sets of marking dots 63 joined by line segments 61 represent simultaneous measurement positions including each of the electrodes 14 of first device 12.

Nearby positions, in some embodiments, are distinguished by their own associated measurements in a fashion which varies continuously so that relatively nearby positions tend to be associated with correspondingly relatively similar sets of voltage measurements. However, relative positions of measurements 61 of measurement cloud 60 may not be initially known. For example, the distribution of electrical field gradients may include significant non-linearities with respect to their corresponding magnitudes and orientations in space. This potentially interferes with the use of electrical field measurements as a direct indication of position; e.g., isopotential lines of electrical fields bend through space, and may be closer or further apart depending on the distribution of structures with different dielectric properties through which the electrical fields extend.

However, position is potentially recoverable by using the measurement values themselves, along with additional information, to produce a reconstruction of the spatial distribution of the electrical fields 62A, 62B, 62C, herein, such a reconstruction is also referred to as a map.

In some embodiments, the rule optionally includes a rule for transforming a voltage and/or impedance measurements cloud (e.g., a group of measurements defined in a space with dimensions corresponding to measurements of different electrical fields and/or by different electrodes) to a position cloud (e.g., positions in a model of three-dimensional physical space associated with the measurements, which comprise a form of structural data). In some embodiments, the rule may include a rule for transforming voltage measurements to positions within the body cavity.

In some embodiments, the rule of block 104 comprises use of such a map.

The map is optionally created using one or more of several available methods. Methods of converting electrical field measurements made by an electrode catheter to a map of positions at which those measurements were made are described, for example, in International Patent Publication No. WO2018/130974 and/or in International Patent Publication No. WO2019/034944, the contents of which are incorporated herein by reference in their entirety. In some embodiments, known distances between electrodes on an electrode catheter or other electrode probe are used to constrain the relative positions at which simultaneous measurements are made. The mutual distance constraint (indicated by the connecting lines 61) provides a kind of “ruler” that allows local scaling to be determined, in some embodiments, even when the change in voltage and/or impedance measured as a function of position offset is variable in magnitude and/or direction. Additionally or alternatively (for example), the problem of non-linearity is managed by computational modeling of the electrical fields, and/or by the conditions of generating the electrical fields: e.g., electrodes are optionally placed at sufficient distances and relative angles relative to the body cavity 49) so that significant portions of the electrical field change about linearly as a function of position offset along some direction.

The rule of block 104, in some embodiments, may be considered equivalent to application of a mapping between values of electrical field measurements made using the first device 12, and corresponding positions in space. Optionally, the rule includes additional features, such as modification for calibration with measurements made by a second device 20 having different electrical measuring characteristics than the electrodes of the first device 12. In some embodiments, the mapping is supplemented by additional information; for example: constraints on where and/or when it is valid, and/or how it may be interpolated and/or extrapolated to positions which were not measured.

Optionally, the measurements from which a rule is generated are “over-determining”—that is, more measurements (e.g., of different electrical fields) are made at each position than are actually needed to uniquely identify positions. This potentially helps in reducing ambiguity and/or error in generating the rule. For example, there may be four or more electrical fields measured, even though the measurement values of only three crossing electrical fields may be sufficient to construct a rule which uniquely determines a position in space. A rule specifying spatial correspondences with just two, or even just one electrical field may be sufficient to determine position in a case where there are other constraints on position. For example, where position varies substantially along a single axis (e.g., because movement is constrained by advance out of or retraction into a catheter tube), position can potentially be determined from knowing the location of the axis in space (e.g., an orientation and a point of intersection), and a single electrical field measurement.

At block 105, in some embodiments, a second device 20 comprising an electrically conductive portion 21 is configured to a configuration which is suited to application of the rule of block 104, when the second device 20 is placed within the body cavity 49 in which the measurements of the first device 12 were made.

To be so-configured, the second device 20 is at least configured to act as an electrode within an electrical field measurement system. In particular, at least a conductive portion 21 of the second device 20 is itself electrically conductive (e.g., comprised of a metal or other low-electrical resistance material), and is furthermore connected to an electrical measuring device via a conductive member.

The conductive member, in some embodiments, is also a structural member to which the second device 20 is mechanically attached. The structural member may comprise, for example, a cable, tube, and/or strut which is operated to mechanically manipulate the second device 20; e.g., to extrude it from an electrically insulating catheter sheath.

The second device 20, in some embodiments, moreover comprises a deploying device; that is, a device which is delivered to the body cavity 49 in a first shape (for example, a shape suitable for movement along the lumen of a catheter tube), and deploys to a second shape (for example, a shape suitable for one or more functions of anchoring, blocking, moving, interconnecting, cutting, restraining, and/or affixing). In some embodiments, the second device 20 itself retains a substantially constant shape (e.g., a linear shape), but is optionally extruded more or less into the body cavity 49 from within a relatively insulating sheath. These changes in shape and/or extrusion distance also affect, in some embodiments, how measurements are made and/or how position-estimation rules are applied.

Two broad approaches by means of which a second device 20 and/or the position estimating system overall can be further configured to be suited to application of the rule of block 104 are (1) using and/or modifying the second device 20 so that it behaves more like electrodes of the first device 12, and (2) using calculations of how different physical and/or electrical characteristics of the second device 20 affect application of the rule of block 104. This is implemented, optionally, as modification of the rule of block 104 itself, and/or as further adjustments made upon application of the rule at block 108.

A third approach is to take measurements under special conditions which can be used to calibrate second device 20 measurements to first device 12 measurements. Any one, two, or three of these broad approaches are optionally used in some embodiments of the present disclosure.

Electrodes of the first device 12 may each comprise, for example, a relatively small ring, e.g., of 1-2 mm diameter and 1-2 mm length (these measurements refer to the portions of the electrode electrically exposed to the environment of the body cavity 49, and exclude, for example, insulated wire conductors used to conduct current to a measurement device). The electrically conductive portion 21 of the second device 20, in contrast, may be larger (e.g., having at least one dimension in a range of 5-30 mm or larger) and/or more complex in shape, potentially resulting in different electrical field measurement properties.

In some embodiments, the second device 20 is made suited to application of the rule by being placed in a configuration selected to mimic the electrical field measurement properties of an electrode of the first device 12. In a relatively simple example, the second device 20 is extruded from an insulating sheath to a small extent, e.g., so that only a portion of about the size of the electrodes of the first device 12 is electrically exposed to the environment of the body cavity 49 within which it is moving.

This “mimicking configuration” potentially helps to reduce complexity and/or increase accuracy of the rule for determining positions of the second device 20, as next explained. FIG. 1D shows an example of a second device 20 in a partially extended position which mimics an electrode 14 of first device 12. FIG. 1E shows the same device 20 in a fully deployed configuration, including an exposed portion of a connecting member 22, which is also a structural member used to advance and/or retract second device 20 relative to catheter sheath 10. The example of device 20 shown in FIG. 1D-1E is an LAA occlusion device (LAAO device), for which a goal of position estimation is to locate a tip of catheter sheath 10 at an ostium 56 of LAA 52 in preparation for deployment of device 20; and optionally another goal of position estimation is to monitor deployment of device 20.

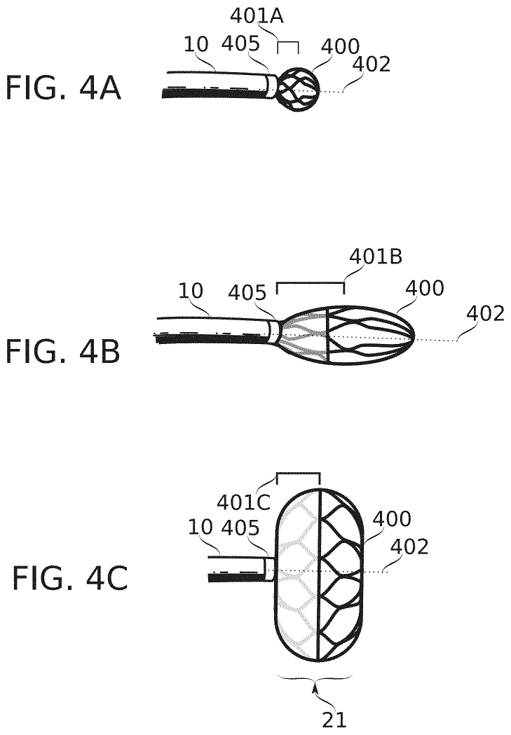

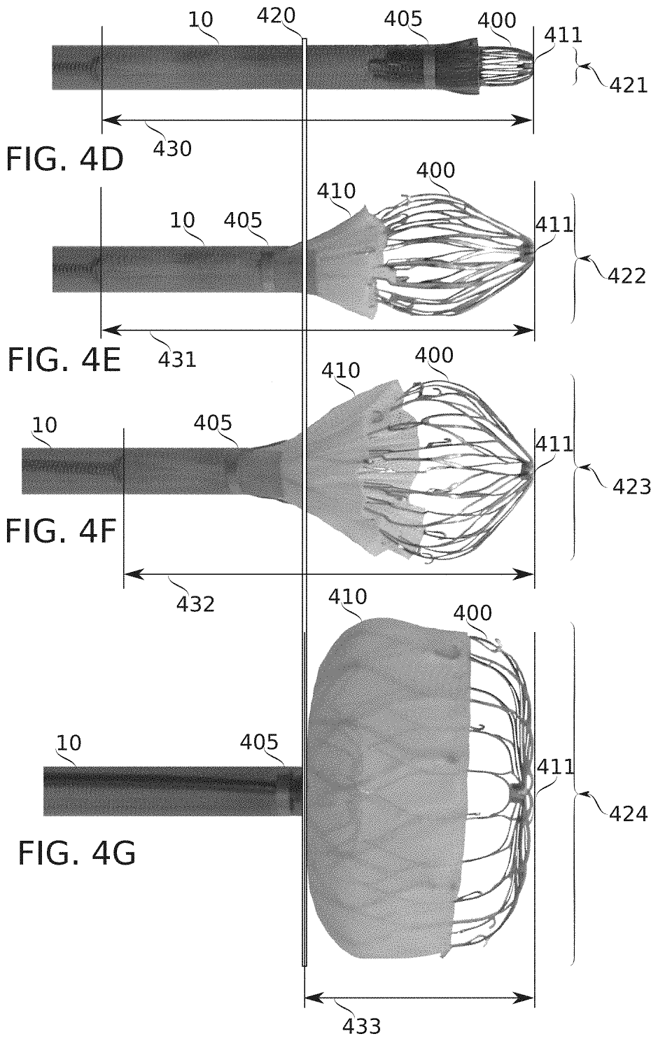

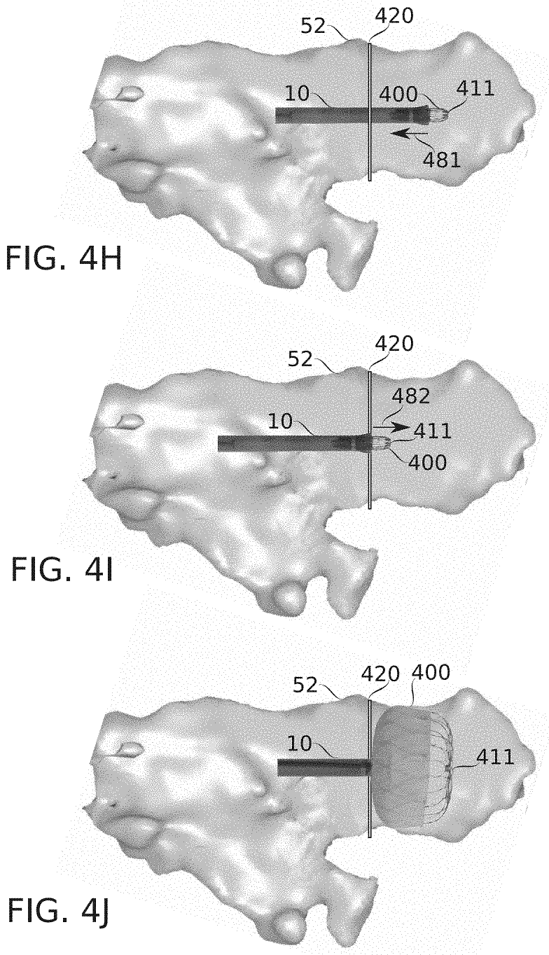

Examples of different devices and corresponding different configurations thereof are discussed, for example, in relation to FIGS. 4A-4G, 5A-5C, and 6A-6C.

When positions of the second device 20 are to be estimated while the second device 20 is not in a mimicking configuration, measurements made by the second device 20 are optionally subjected to further processing.

At block 106, in some embodiments, second-device measurements are received. At block 108, in some embodiments, the rule of block 104 is applied (optionally with additional corrections, for example, as next described; depending on whether the rule of block 104 is modified on the fly according to conditions of calibration and/or second-device configuration). At block 110, in some embodiments, the second-device position estimate is provided. In some embodiments, the second-device position estimate may be used for navigating the second device 20 within the body cavity, for example: during a medical procedure.

The operations of block 108-110 are optionally implemented by a rule as simple as a lookup performed on the map of first-device measurements to first-device position, using instead the second-device measurements of block 106. This is particularly well-suited to embodiments of the method wherein the second device 20 is operated in a mimicking configuration.

Alternatively, the rule includes additional corrections to adjust for differences between the second device 20 and the first device 12.

The electrically conductive portion 21 of the second device 20 is effectively at a single voltage potential (disregarding minor internal resistances and reactivity). More particularly, it is measured as having a single potential at any given time by the electrical measuring device to which it is connected. Accordingly, the second device 20, even when fully expanded, can be understood as having an equivalent first-device electrode position, given by applying the rule generated from first-device measurements to second-device measurements.

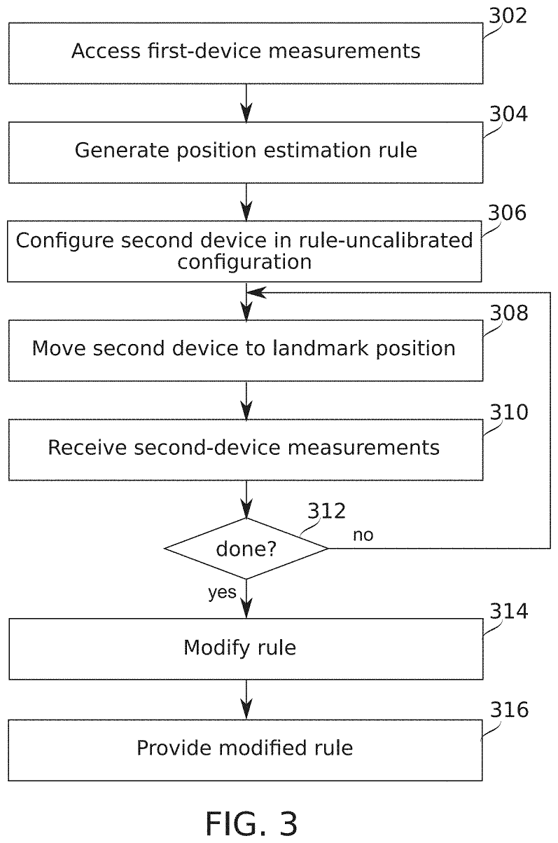

Second-device measurements of different electrical fields may, however, associate to different corresponding equivalent first-device electrode positions (even if measured simultaneously). This potentially complicates position determination, insofar as a set of simultaneous measurements made using the second device 20 may not, in fact, fully correspond to a measurement set made at any particular position of the first device 12. In some embodiments, this is overcome by calibration (methods of calibration are discussed herein, for example, in relation to FIG. 3). In some embodiments (with or without calibration), the rule of block 104 is adjusted from the basis of an initial lookup into the map of first-device measurements to first-device positions. For example, a measurement set made using the second device 20 is first associated to the closest available first-device measurement set (e.g., by a sum of absolute magnitudes of the difference, by least mean squares, and/or by another metric), and then to a position offset from the map position according to a calibration function that relates first-device positions to second-device positions.

The equivalent first-device electrode position of the second device 20 is likely to be within (or at least nearby) an envelope defined by the larger physical extent of the second device 20.

Accordingly, in some embodiments, the position of the second device 20 is also defined as an envelope around the equivalent first-device electrode position (this can be implemented as a suitable modification to the rule of block 104). This allows estimation, e.g., of distances to contacts with walls of the tissue cavity, even though the equivalent first-device electrode position itself never reaches such a contact.

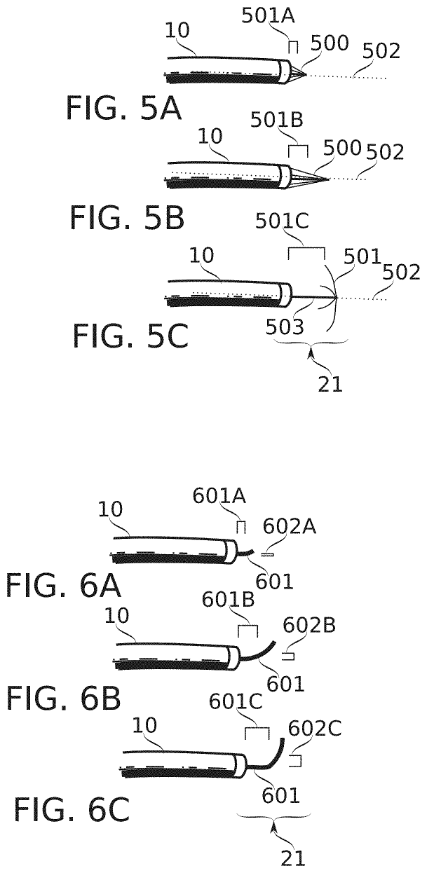

For second devices 20 that undergo a conformational change during use (e.g., expansion during deployment): both the envelope, and its relationship to the equivalent first-device electrode position, are optionally varied as a function of shape. In some embodiments, these variations are made part of the rule of block 104. Different methods may be used to estimate a relationship between a second-device shape envelope and the equivalent first-device electrode position. In some simple implementations, a geometric center of the second device 20 (e.g., of its envelope, and/or of its center of mass) is assigned as the equivalent first-device electrode position. Optionally, interaction of the second device 20 with the electrical fields is electrically modeled, which can take into account, e.g., effects of electrical field gradient non-linearities, and/or influences of the second device 20 itself on the distribution of electrical field potentials. Conversely, the behavior of the equivalent first-device electrode position as a function of manipulation of the second device 20 is optionally used as an indication of second-device state, for example as described in relation to FIGS. 4A-4G, 5A-5C, and/or 6A-6C, herein.

From block 112, in some embodiments, further measurements, rule applications, and position estimates may be performed until estimating of the position of the second device 20 ends. The second device 20 may be placed into different configurations in different loops, which can affect, for example, how the rule is applied at block 108.

### Second Device Navigation in Two Configurations

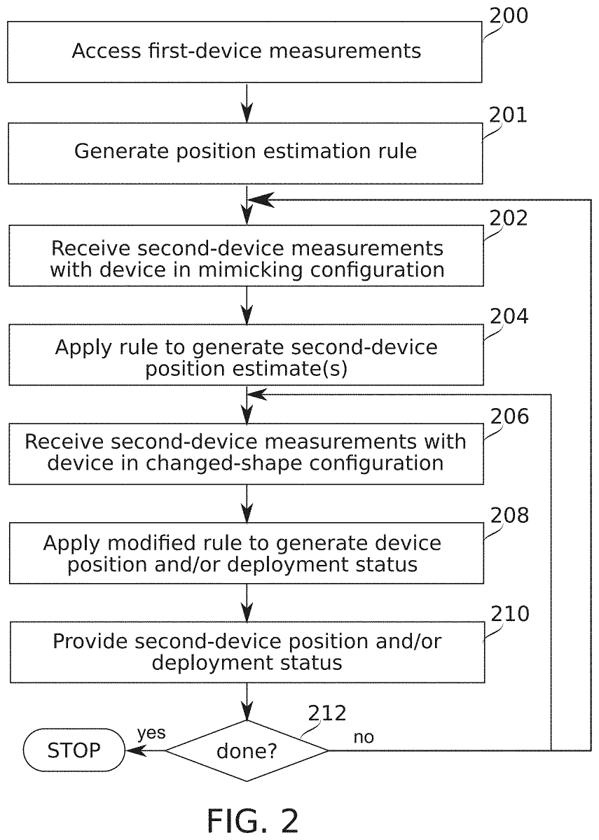

Reference is now made to FIG. 2, which is a flowchart schematically illustrating a method of positioning a second device 20 using a combination of a mimicking configuration measurements and changed-shape configuration measurements, according to some embodiments of the present disclosure. In some embodiments, the method of FIG. 2 is more particular example of the method of FIG. 1A.

At block 200, in some embodiments, first-device measurements are accessed, and at block 201, a position estimation rule is generated. These blocks correspond, in some embodiments, to blocks 102 and 104 of FIG. 1A. In some embodiments, the method begins with accessing (and not necessarily with the generating of) a position estimation rule. The position estimation rule may, for example, have been previously generated from measurements which were obtained from a first device 12 comprising a plurality of electrodes configured to measure electrical fields generated to extend through the body cavity 49.

At block 202, in some embodiments, second-device measurements are received, with the second device 20 in a mimicking configuration (for example, as described in relation to FIG. 1D and/or block 105 of FIG. 1A). At block 204, in some embodiments, the rule of block 201 is applied to generate a position estimate for the second device 20.

At block 206, in some embodiments, second-device measurements are received, with the second device 20 in a changed-shape configuration (for example, as described in relation to FIG. 1E and/or block 105 of FIG. 1A). At block 208, in some embodiments, a modified rule based on the rule of block 201 is applied to generate a position estimate for the second device 20. The rule may be modified to a compensate for a difference in the shape of the device at block 206 as opposed to the block 202—for example, an offset may be added to the rule, and/or a change in “gain”. At block 210, the second-device position and/or deployment status is provided.

From block 212, in some embodiments, further measurements, rule applications, and position estimates may be repeated until estimating of the position of the second device 20 ends. The repeating may include a return to block 202, or to block 206. The two alternative for the repeating are distinguished in that blocks 202 and 204 are performed with the device 20 in a different configuration than blocks 206 and 208.

In some embodiments, an additional distinguishing feature is that the type of motion for which position estimations are generated is different. In some embodiments, movements during the position estimation in blocks 204 and 206 are free in three dimensions. This may be suitable, for example, for positioning to reach the deployment-ready position shown in FIG. 1D. Since second device 20 is in a mimicking configuration, its position can be determined using a position-estimating map as if it carried an electrode like that of the first-device measurement device.

In some embodiments, position finding in blocks 206 and 208 is optionally performed while motion is constrained to operations which alter a deployment status of second device 20: for example, extrusion of second device 20 from catheter sheath 10 by advancing of connecting member 22, and/or actuation/deployment by other methods and/or control members. In some embodiments, second device 20 is elastically biased to self-expand or otherwise change shape as constraint from catheter sheath 10 is removed.

In cases where motion is limited to deployment movements, the position last estimated at block 204 is used, in some embodiments, as a base position. Subsequent position estimates are made as estimates of change from this position. For example, as second device 20 is extruded from catheter sheath 20, its equivalent first-device electrode position also advances, at least initially. With further expansion, there can also be a reversal of the direction of motion of the equivalent first-device electrode position, or another motion; for example, as described in relation to FIGS. 4A-4G, 5A-5C, and/or 6A-6C. Even though the overall shape and/or absolute position of second device 20 is not recorded by the measurements directly, it can optionally be calculated, with the understanding that movement of the equivalent first-device electrode position reflects a change in deployment status (shape) along a constrained range of possibilities. The movement can be understood in different ways, depending on the conformational changes undergone by the second device 20 as it deploys, for example as described in relation to FIGS. 4A-4G, 5A-5C, and/or 6A-6C, herein.

Additionally or alternatively, in some embodiments, interconversion between a mimicking configuration and a changed-shape configuration is performed several times, at different positions. Optionally, the positions of interconversion are used as calibration anchoring locations, allowing a rule which generates first-device positions from first-device measurements to be transformed (e.g., by interpolation between calibration anchoring locations) to generate second-device positions from second-device measurements, even when the second device 20 is in a configuration which does not mimic first-device electrodes. The calibration, in some embodiments, is between second-device measurements and first-device measurements; determining, e.g., adjustments in offset and/or scale of second-device measurements so that they can be used like first-device measurements in determining device positions.

### Second Device Measurement Calibration to First Device Measurement/Position Rules

Reference is now made to FIG. 3, which is a flowchart schematically illustrating a method of calibrating electrical field measurements made using a second device 20 within a body cavity 49 to electrical field measurements made using a first device 12 within the body cavity 49, according to some embodiments of the present disclosure.

The method of FIG. 3 shows a method of obtaining calibration anchoring locations, in which the position of the second device 20 is known from information other than its own electrical field measurements. This information is used to assign the electrical field measurements obtained using the second device 20 to a position, and through this assignment, determine what measurements the first device 12 would have made at the same position. From using several such calibration anchoring locations it can be determined how first-device electrical field measurements at that same position vary from the second-device electrical field measurements. Even two calibration anchoring positions are potentially sufficient e.g., to set a calibration scaling factor and/or offset along an axis of motion. As more calibration anchoring positions are obtained, the quality of the calibration is potentially improved.

The calibration, in some embodiments, is between second-device measurements and first-device measurements; determining, e.g., adjustments in offset and/or scale of second-device measurements so that they can be used like first-device measurements in determining device positions. Optionally, the calibration anchoring locations of the method of FIG. 3 are used along with calibration anchoring positions determined as described in relation to FIG. 2 to produce a calibration of second-device measurements to first-device measurements.

At block 302, in some embodiments, first-device measurements are accessed, and at block 304, a position estimation rule is generated. These blocks correspond, in some embodiments, to blocks 102 and 104 of FIG. 1A. In some embodiments, the method begins with accessing (and not necessarily with the generating of) a position estimation rule. The position estimation rule may, for example, have been previously generated from measurements which were obtained from a first device 12 comprising a plurality of electrodes configured to measure electrical fields generated to extend through the body cavity 49.

At block 306, in some embodiments, a second device 20 is configured to a “rule-uncalibrated” configuration; that is, it is deployed into a shape which does not mimic an electrode of the first device. At block 308, the second device 20 is moved to a landmark position. This can be, for example, a position of catheter access into the body cavity 49 (e.g., a fossa ovalis or other point of transseptal penetration), and/or a position of farthest travel (e.g., a place at which advancement of the second device 20 is blocked by an encounter with a lumenal wall).

At block 310, in some embodiments, second-device measurements are received, which allows defining of a calibration anchoring position. From block 312, in some embodiments, the collection of calibration anchoring positions continues with repetition of block 308 and block 310 as necessary. Optionally, measurements at positions between well-defined landmarks (or offset from a single landmark in a known direction) are also collected for use as calibration anchoring positions, with their own positions being calculated according to the distance of advance along the path of travel. Once enough calibration anchoring positions have been collected, the method continues with block 314, wherein the position estimation rule of block 304 is modified, by use of the collected calibration information. At block 316, in some embodiments, the modified rule is provided. The modified rule becomes an optional basis for further position estimations, for example as described in relation to FIG. 1A, herein.

### Second Device Examples

Cage Shaped Device—LAAO Device

Reference is now made to FIGS. 4A-4C, which schematically illustrate movement of an equivalent first-device electrode position of a cage-shaped second device as the second device deploys, according to some embodiments of the present disclosure. In some embodiments, the cage-shaped device is a left atrial appendage occluder device (LAAO device), for example as described in relation to FIGS. 1D-1E.

In each of FIGS. 4A, 4B, and 4C, a second device 400 is shown in a different state of deployment (shape) as it is extruded from a catheter sheath 10. It should be understood that the deployment states of the device itself are the same in case of withdrawing catheter sheath 10 rather than advancing the second device 400. In some embodiments, second device 400 corresponds to the example of second device 20 illustrated, e.g., in FIGS. 1D-1E. Second device 400 corresponds, in some embodiments, to an LAA closing device. Such devices are used to close off the lumen of LAA from the general circulation. This potentially prevents blood clots which may form within the relatively static flow environment of the LAA from dislodging and entering the general circulation where they can create blockages and corresponding ischemia. In some embodiments, second device 400 is a self-expanding device; e.g., comprising nichrome struts compressed to a delivery configuration, and self-expanding once freed from confinement within catheter sheath 10.

In FIG. 4A, second device 400 is deployed by a small amount. Bracket 401A shows an estimated distance of the equivalent first-device electrode position from a distal tip of catheter sheath 10; equal to about half the overall distance of second device 400's deployment, or about where the center of gravity of the unsheathed and electrically conductive portion 21 of second device 400 is located. Local anisotropies in the electrical field environment may modify this estimate somewhat. In some embodiments, the modification is within a range small enough to be disregarded. Optionally, the modification is calculated and corrected for using the previously determined map of first-device electrical field measurements to positions.

For purposes of illustration, it may be presumed that the equivalent first-device electrode position is located along a central longitudinal axis 402 defined by the orientation of the distal tip of catheter sheath 10. However asymmetries of the second device 400 and/or the electrical field environment can potentially draw the equivalent first-device electrode position away from this central axis.

In FIG. 4B, deployment of second device 400 has proceeded further, and bracket 401B shows a corresponding increase in the distance of the equivalent first-device electrode position from the distal tip of the catheter sheath 10.

In FIG. 4B, second device 400 fully freed from within catheter sheath 10. As second device 400 finished expanding in radial directions, it also shortened again, causing its equivalent first-device electrode position to draw back toward the distal tip of catheter sheath 10. Bracket 401C indicates this shortened distance compared to bracket 401B. This forward-then-backward movement is potentially characteristic of deployment for devices of this type, and is optionally used as a marker to help a physician track the stage of deployment. For example: before the reversal, the physician can be reasonably confident that the device is collapsed enough to allow easy repositioning; while after the reversal, the physical can be reasonably confident that the device is expanded enough to anchor.

Reference is now made to FIGS. 4D-4G, which schematically illustrate conformational changes of a cage-shaped second device as the second device deploys, according to some embodiments of the present disclosure. In some embodiments, the cage-shaped device is a left atrial appendage occluder device (LAAO device), for example as described in relation to FIGS. 1D-1E.

The sequence of FIGS. 4D-4G shows sheath 10 (bearing an electrode 405) being withdrawn from around a device 400 (corresponding, in some embodiments, to an LAAO device), as a distal tip 411 of device 400 remains stationary. Even when it is the sheath moving (e.g., while the device 400 remains stationary), this movement is still referred to herein as “advance” of the device from the sheath (that is, the “advance” refers to their relative motion), and the same deployment configurations of the device itself are obtained whether it is the sheath 10, the device 400, or both which are moving. Overall lengths 430, 431, 432, 433 of device 400 change during the sequence. Impedance (not shown) generally drops, and an electrical center of the device moves, for example, as explained in relation to FIGS. 4A-4C. Device 400 also changes through different widths 421, 422, 423, 424 during deployment (corresponding, e.g., to about 4 mm, 8 mm, 12 mm, and 24 mm, respectively). While device length and/or width is optionally not directly measured, it may be inferred; e.g., by using previously measured associations of certain degrees of advance and/or certain impedances with certain device shapes.

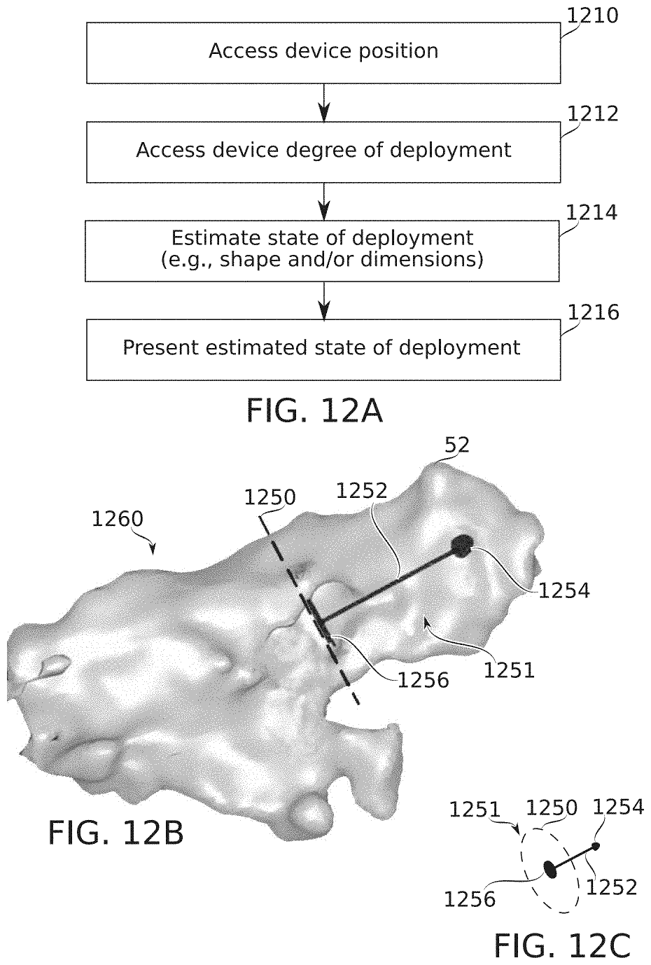

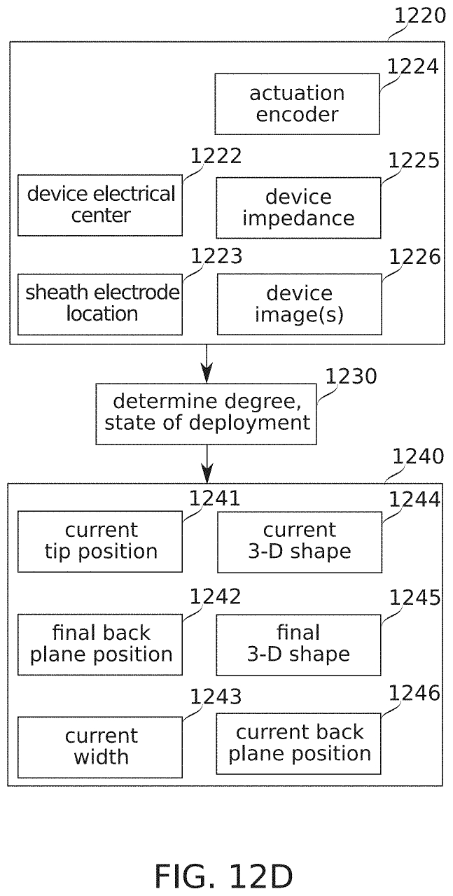

Also shown in the sequence is the expansion of netting 410. Backplane 420 is also indicated; adjacent to the proximal-most position along a proximal-distal axis which device 400 will occupy when fully deployed. It is noted that as long as distal tip 411 remains stationary, the position of backplane 420 can be estimated before deployment actually occurs. Herein, the term “back plane” refers to a plane transverse to a proximal-to-distal axis of a distal tip of a device delivery sheath 10 which intersects and/or is tangent to the most portion of a device 400 at some stage of deployment. Unless stated otherwise, the stage of deployment is completed deployment of the device 400. Presentation of the current and/or anticipated position of backplane 420 is discussed, for example, in relation to FIG. 12B, herein.

Reference is now made to FIGS. 4H-4J, which schematically represent sheath-withdrawal (FIG. 4H) and device-extruding (FIG. 4I) deployment of an LAAO device 400 to the deployed state of FIG. 4J, according to some embodiments of the present disclosure.

FIG. 4H shows an early position of sheath-withdrawal type deployment, with the tip 411 of LAAO device 400 initially placed relatively deep within LAA 52 in order to achieve deployment against a targeted backplane 420. Deployment continues with withdrawal of sheath 10 in the direction of arrow 481, until device 400 is allowed to fully expand within the available confines of LAA 52 (FIG. 4J; in FIG. 4J, relative to FIG. 4H, the position of sheath 10 represents a slight re-advance after deployment). A potential advantage of this mode of deployment is that the available depth for deployment can be fully confirmed by placement of the device tip 411 before deployment begins, since tip 411 will advance no further into the LAA during deployment.

FIG. 4I shows an early position of device-extrusion type deployment, with the tip 411 of LAAO device 400 initially placed at a relatively shallow position within LAA 52, with a distal tip of sheath 10 positioned at a targeted backplane 420. LAAO device 400 is then advanced in the direction of arrow 482 until device 400 is fully expanded at the position shown in FIG. 4J. A potential advantage of this mode of deployment is that deep penetration into the LAA only occurs while device 400 is mostly expanded. This is a potential advantage for reducing a risk of dislodging clotted blood into the circulation, since a partially loosened thrombus may still be retained by the mostly expanded body of LAAO device 400. However, a maximum distance of penetration into the LAA may be slightly greater than is the case for sheath-withdrawal type deployment, during a period when the device 400 is mostly extruded, but still has not fully expanded.

Optionally, the two modes of deploying are combined to obtain all or part of the relative potential advantages of each (e.g., early deployment is by device extrusion; later deployment is by sheath withdrawal). This, however, potentially requires judgement and/or measurement of the moment to switch between modes, so that final deployment of device 400 is in the correct position relative to targeted backplane 420.

Method of Showing Device Deployment

Reference is now made to FIG. 12A, which is a schematic flowchart of a method of showing a device deployment state based on measurements of its position, and parametric measurement of the device's degree of deployment, according to some embodiments of the present disclosure. The device, in some embodiments, corresponds to the “second device” 400, e.g., the left atrial appendage occluder device of FIGS. 4A-4G.

Reference is also made to FIG. 12D, which schematically represents data sources 1220 which optionally provide parametric indications of degree of device deployment, and aspects of the state of deployment 1240 determined from the parametric indications according to some embodiments of the present disclosure. In some embodiments, a current and/or predicted state of deployment of the device is presented to an operator—wherein the state of deployment presented comprises an indication of the device's current and/or predicted dimensions and/or shape.

At block 1210, in some embodiments, a position of device is accessed. The accessed position is obtained, for example, by position sensing using the device as an electrode, and/or using an electrode co-located with the device, for example, an electrode 405 on a catheter sheath 10 which delivers the device. Optionally, any other position-determining method is used, e.g., magnetic tracking, ultrasound imaging, and/or X-ray imaging.

At block 1212, in some embodiments, information about a degree of deployment of the device is accessed, to provide a parametric measure of the device's degree of deployment. Data sources 1220 (FIG. 12D) represent some of the optional sources of data which may be used to determine a degree of device deployment 1230.

In some embodiments, degree of deployment corresponds to an advance distance of the device out of the catheter sheath.

In some embodiments, advance distance is measured using movements of a device electrical center 1222 (e.g., the movement of an “equivalent first-device electrode position” of an LAAO device). For example, if a deployment sequence such as is shown in FIGS. 4D-4G is used, the device electrical center 1222 will appear, for some stages of deployment, to move proximally as catheter sheath 10 is pulled back proximally (e.g., between positions of FIG. 4D and FIG. 4E; and between positions of FIG. 4E and FIG. 4F). Additionally or alternatively, the parametric measure of the advance distance corresponds, in some embodiments, to the lengths of brackets 401A, 401B, 401C. Since the same bracket length may correspond to more than one deployment state (e.g., if the device electrical center 122 reverses course mid-deployment, as shown between FIGS. 4B-4C), the parametric measure is optionally supplemented by additional information. The additional information comprises, for example, a time history of how the parametric measure has been changing; and/or information about the impedance 1225 of the device, which may be lower in a more expanded state than in a less expanded state sharing the same apparent advance distance. In some embodiments, device impedance 1225 alone is sufficient to indicate a degree of device deployment.

Additionally or alternatively, a measure of the state of deployment is based on deployment-triggering actuation, such as movement of a deploying control which is actuated to advance the device, and/or change a shape of the device upon advancing from its delivery sheath. In some embodiments, the advance distance is measured using an output of an actuation encoder 1224 which encodes, for example, a distance a control member is moved.

Optionally, one or more images 1226 of the device provides information about the degree of deployment; for example, based on distances in an X-ray image of radioopaque markers on the device and/or a delivery sheath for the device. Foreshortening is optionally determined, for example, based on the apparent distance of markers at a fixed distance from each other.

In some embodiments, relative positions and/or distances of a plurality of electrodes are compared to estimate a degree of deployment of the device. For example (referring to FIG. 4A-4C), a position 1223 of electrode 405 and an equivalent “first-device electrode position”/device electrical center 1222 (described above) of second device 400 are compared. In some embodiments, what is measured and used as an estimate of deployment state is the (e.g., electrically measured) change in position of the device from an initial degree of deployment (e.g., the state of FIG. 4A) to its current degree of deployment (e.g., as illustrated in FIGS. 4B-4C). This estimate is optionally corrected for movements of the whole catheter, for example, by tracking the catheter position separately from a position of the device.

At block 1214, in some embodiments, a state of deployment (e.g., the dimensions and/or shape of the device) corresponding to the parametric measure(s) of the device's degree of deployment is estimated. The corresponding state of deployment is optionally estimated by selection from a set of predetermined associations of modeled and/or measured shapes (such as the shapes of second device 400 in FIGS. 4A-4C) to different parametric measures of deployment (e.g., a degree of advance and/or another actuation). Optionally, the set of predetermined associations encodes the states of deployment as distinct images, measurement sets and/or shape models of the device. Optionally, the association is through a dynamic model of the device which is itself parametrically defined to set a particular state of deployment. Blocks 1212 and 1214 together correspond to block 1230 of FIG. 12D.

At block 1216, in some embodiments, the state of deployment is presented to the operator, for example as an image of the device as it is currently estimated to exist in situ, and/or as a schematic representation such as the schematic representation 1251 of FIG. 12B. The device can be presented, for example in its current estimated 3-D shape 1244, in an estimated final 3-D shape 1245 (i.e., the shape of the device estimated if it were deployed at the current position), a current tip position 1241, a current width 1243, a current backplane position 1246 (before deployment completes, this is optionally a position of the distal tip of the sheath 10) and/or a final backplane position 1242 (for example, as described in relation to FIG. 12B). In some embodiments, the presentation is live-updating: when an operator moves the device position and/or changes its state of deployment, and the state of the deployment presented changes correspondingly.

It should be noted that use of the position accessed at block 1210 is optional (as is block 1210 itself). In some embodiments, the presentation of block 1216 omits showing a position of the device relative to its surroundings, and simply shows an estimated state of deployment, e.g., an image similar to one of the deployment states of second device 400 shown in FIGS. 4A-4C, or another image. That image can be presented, in some embodiments, separately from a presentation of the model.

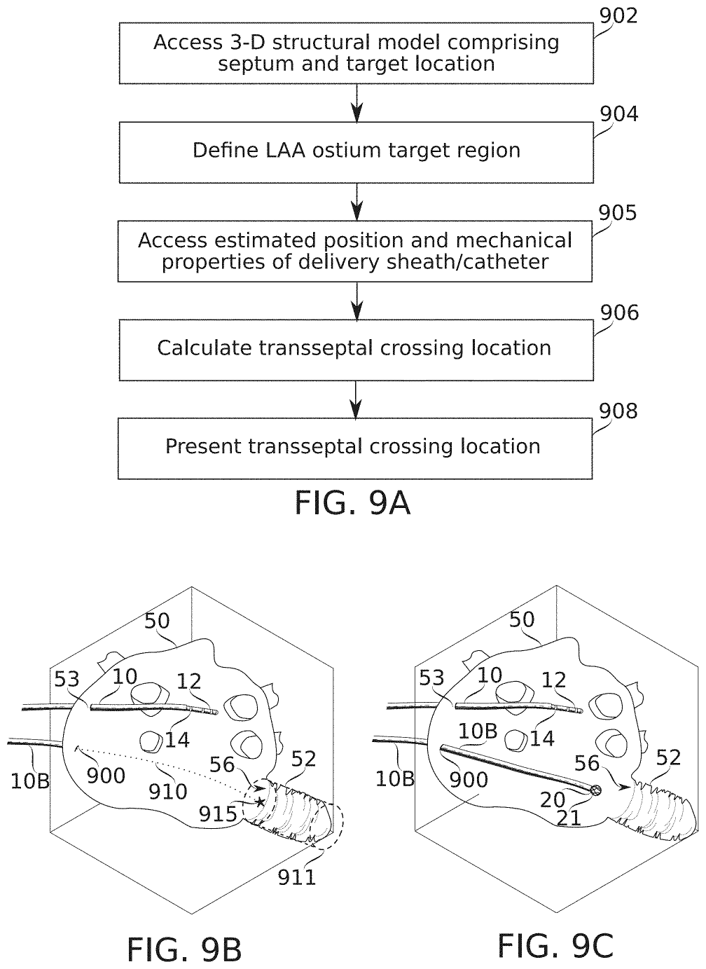

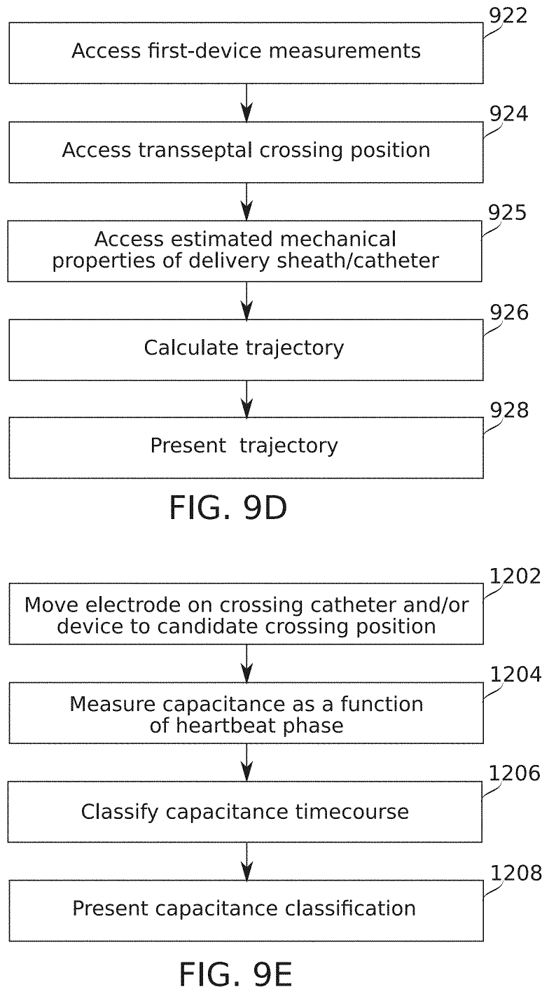

It should also be noted that the parametric degree of device deployment accessed at block 1212 is not necessarily a measured degree of deployment. In some embodiments, an operator may select (e.g., by use of a user interface) viewing how a device is estimated to appear at a particular position (e.g., a current position) if it was deployed at that position (e.g., partially or fully deployed). The position is not necessarily the current position; for example, the position is optionally a position at an end of a trajectory of a deployment sheath and/or device. The trajectory is optionally calculated and/or presented as described in relation to FIGS. 9A-9D, herein. Optionally, the presented state of deployment takes into account interference with nearby tissue walls which a device expanded from the current or selected position would encounter. For example, the presentation of the device is modified to estimate how the tissue walls would deflect it upon expansion.

Reference is now made to FIG. 12B, which illustrates a presentation of an estimated state of deployment of a LAAO device, according to some embodiments of the present disclosure. Reference is also made to FIG. 12C, which illustrates a schematic LAAO device representation 1251 from an oblique viewing angle, according to some embodiments of the present disclosure.

Partial heart image 1260, in some embodiments, comprises a view of a 3-D structural model of a portion of a heart, including left atrial appendage 52.

In some embodiments, a position of an LAAO device relative to LAA 52 is represented schematically; e.g., as schematic LAAO device representation 1251. Representation 1251 represents the position of a device tip by tip mark 1254, and a (current, e.g., partially deployed) device width by the length of a width mark 1256. Axis mark 1252 indicates the position of a central axis of the LAAO device, extending proximally from the tip mark 1254.

Base plane mark 1250 indicates an estimated position of a proximal side of the LAAO device upon full deployment, while tip mark 1254 remains in the current position. Optionally, base plane mark corresponds to the position of base plane 420, for example as described in relation to FIGS. 4D-4G, herein. Optionally, base plane mark 1250 is selectably shown upon a command from the device operator.

In some embodiments, a size of the indication of base plane mark 1250 indicates how wide the fully deployed device would be if it deployed in an unconstrained space. For a closure device such as an LAAO device, it is a potential advantage to see that the unconstrained closure device is somewhat larger in diameter than the aperture which it is to close off. When it actually deploys to a fully closing state, the device is optionally expected to stretch tissue and/or be compressed by the tissue; if it does not have an unconstrained shape big enough for this, then selection of a device with a larger size may be indicated.

Schematic LAAO device representation 1251 is shown from a full side view in FIG. 12B. A full side view (that is, a view from an angle orthogonal to axis mark 1252) has a potential advantage of illustrating LAAO dimensions without distortion due to foreshortening. In a 3-D view from another angle, base plane mark 1250 and/or width mark 1256 optionally appear as (e.g., foreshortened) disks, for example as shown in FIG. 12C.

Multi-Direction Viewing of Device Status

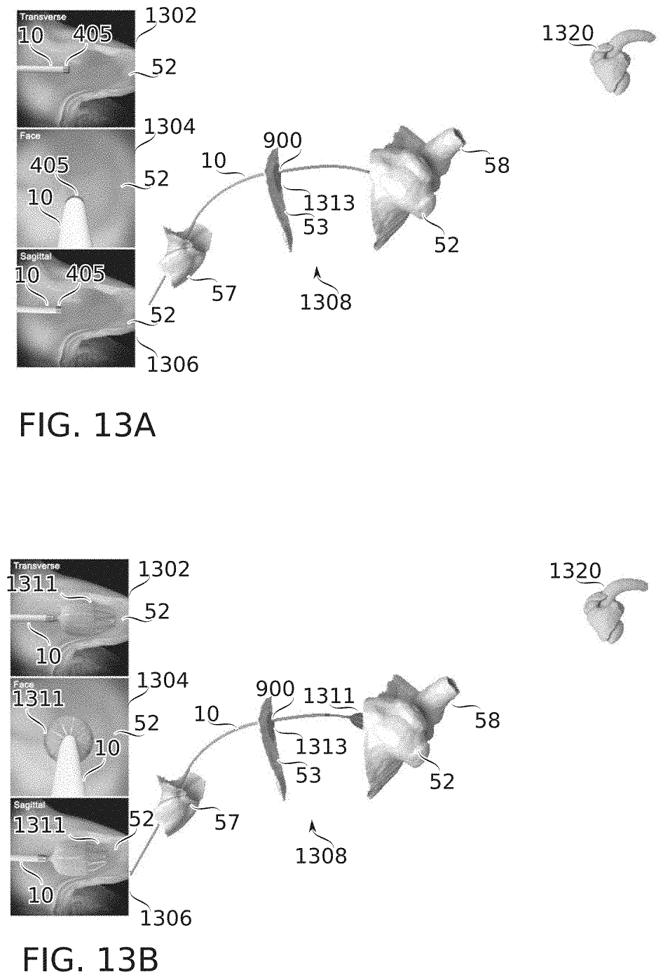

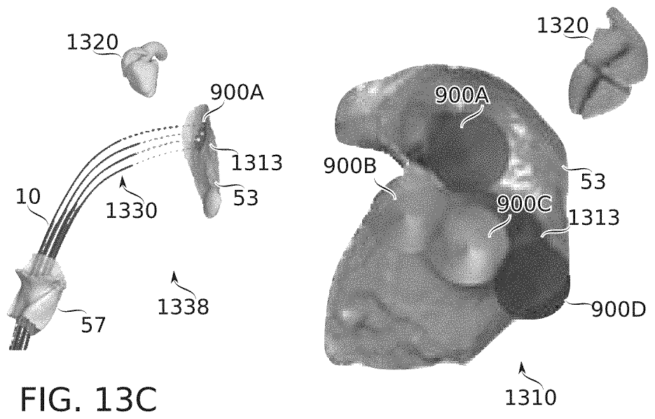

Reference is now made to FIGS. 13A-13B, which represent four-angle views of a 3-D scene presenting positioning (FIG. 13A) and deployment (FIG. 13B) of an LAAO device 1311, according to some embodiments of the present disclosure.

In some embodiments, LAAO device 1311 is a second device such as described, for example, in relation to second device 20 of FIGS. 1D-1E and/or device 400 of FIGS. 4A-4C. In some embodiments, presentation of a current device positioning and/or state of deployment is provided on a display from a plurality viewing angles simultaneously. FIGS. 13A-13B illustrate the use of four views—transverse view 1302, face view 1304, sagittal view 1306, and external perspective view 1308. All views are synthesized, in some embodiments, using a 3-D structural model of portions of a heart; and position and deployment data for LAAO device 1311 and/or catheter sheath 10, for example as described in relation to FIG. 12A.

Face view 1304 is useful, for example, in aiming a motion of the catheter 10. If the motion occurs straight along the body of the catheter sheath 10, the region which can be seen beyond the tip of catheter sheath 10 and/or LAAO device 1311 is the position to which it will move upon further advance. Alternatively, if the motion is curved (e.g., because external forces acting on the catheter 10 cause it to bend), forward motion will generate a shift in field of view which can be noted and corrected for as necessary.

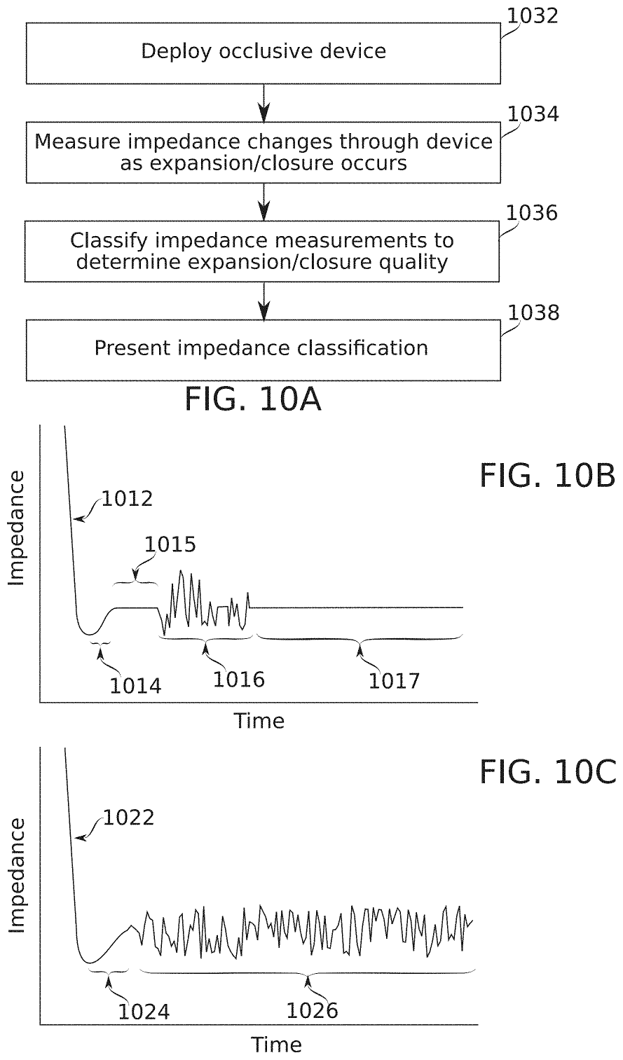

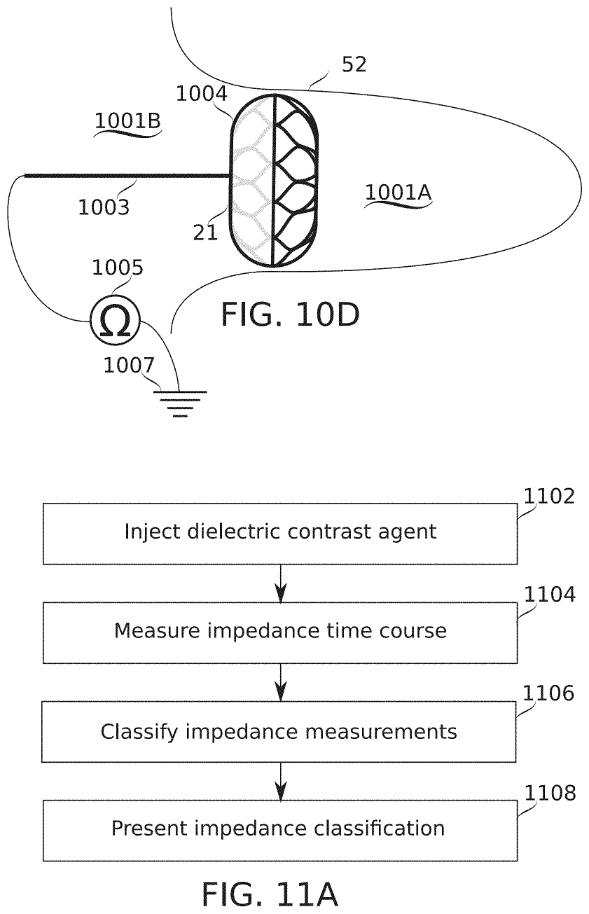

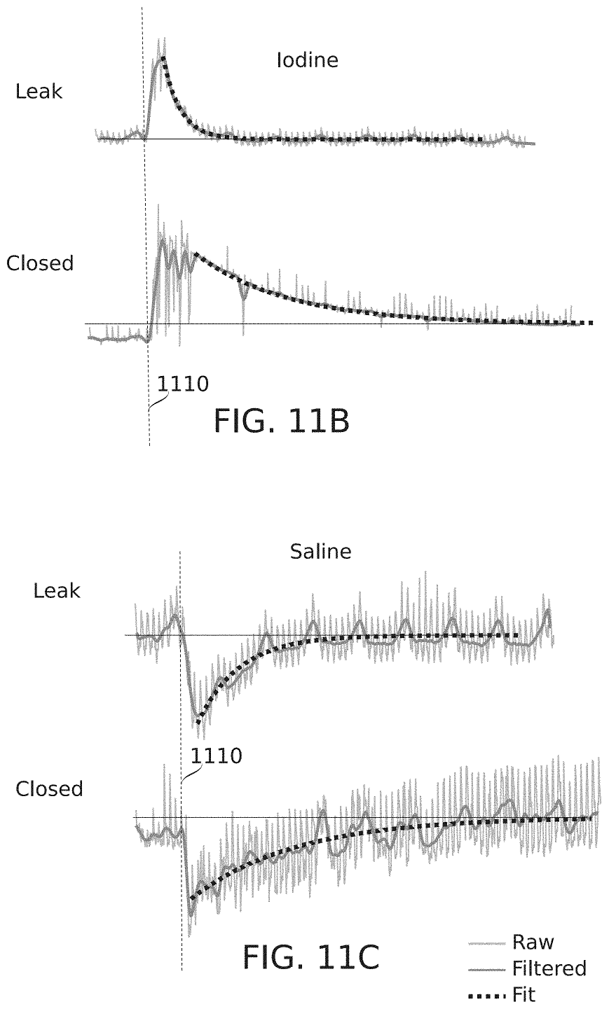

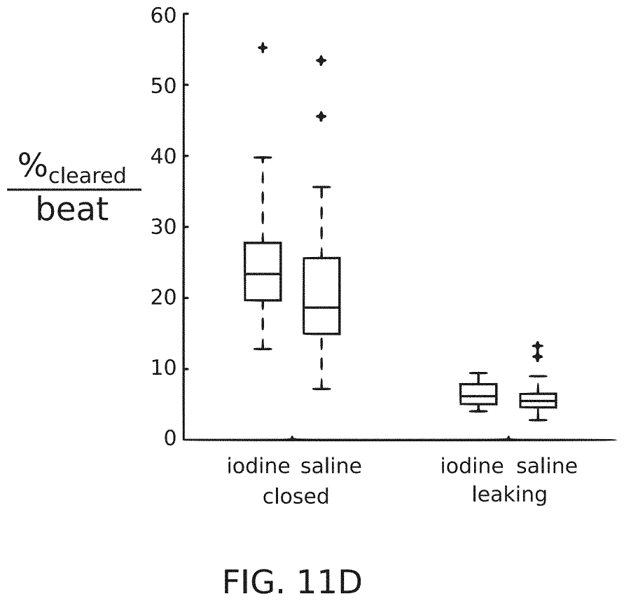

The transverse view 1302 and sagittal view 1306 together are useful in particular, e.g., as references for the positioning (centering) of catheter sheath 10 in LAA 52 before deployment (FIG. 13A). They show the scene from orthogonal angles, and help to overcome uncertainty due to perspective parallax which face view 1304 alone may introduce. Views 1302, 1306 are also useful for presenting the progress of device deployment; and optionally for presenting information indicative of LAA closure by LAAO device 1311, for example information as described in relation to FIGS. 10A-11D, herein.

External perspective view 1308, in some embodiments, comprises a partial view of heart structures associated with the deployment of LAAO device 1311, for example as waypoints (e.g., inferior vena cava 57, and septal wall 53 including crossing location 900 of fossa ovalis 1313, marked in a darker shade), and/or as a target region for device deployment (e.g., LAAO 52). Pulmonary vein 58 is also shown. This fragmented 3-D view provides some potential advantages; for example, it removes distracting features unrelated to navigation, and moreover can be presented even if structural data showing the overall shape of the navigated space is unavailable. Optionally an overview image 1320 of the heart or a portion thereof is shown to help orient the operator to the perspective shown in view 1308. Optionally, external perspective view 1308 (optionally along with overview image 1320) is shown without any of the other views, or with fewer of the views, for example as shown in FIG. 13C.

It should be understood that in some embodiments, a computerized system is provided comprising processor, memory instructing the processor, and display; wherein the memory instructs the computer to carry out the generation of images as described in relation to FIGS. 13A-13B, as well as presentation of the images on the display.

Reference is now made to FIGS. 5A-5C, which schematically illustrate movement of an equivalent first-device electrode position of an “umbrella”-shaped second device 500 as the second device deploys, according to some embodiments of the present disclosure.

The device of FIGS. 5A-5C is radially symmetric, like that of FIGS. 4A-4C, but with a different shape. The device shown has a central strut 503 with secondary struts 501 that expand to umbrella-like configuration. This could be, for example, an anchoring device (e.g., expanding after penetration of a lumenal wall by the central strut), and/or a device for deploying sensing nodes and/or ablation terminals. Regardless of function, second device 500 differs also from second device 400 during deployment in that it shows a different “final expansion” behavior. As secondary struts 501 are freed enough from catheter sheath 10 to expand, they tend to draw the equivalent first-device electrode position rapidly forward. Compare, for example, brackets 501A and 501B (wherein the equivalent first-device electrode position is about half the total advance of the second device 500) to bracket 501C, wherein the expanded struts have drawn the electrical center of the second device 500 much closer to its distal end.

Similarly to the situation in FIGS. 4A-4C, the equivalent first-device electrode position of second device 500 is expected to lie on or close to longitudinal axis 502. Any substantial deviation from this during deployment might indicate a problem, for example, an impediment to arm expansion, for which problem a physician may choose to take corrective action.

Reference is now made to FIGS. 6A-6C, which schematically illustrate movement of an equivalent first-device electrode position of a bent-linear second device 601 as the second device deploys, according to some embodiments of the present disclosure.

The device of FIGS. 6A-6C unsheathes into a bent-linear shape. Brackets 601A-601C show longitudinal advance of the equivalent first-device electrode position, substantially as described for the other devices, except that there is no position of sudden increase or reversal.

Brackets 602A-602C show increasing offset during unsheathing from a central longitudinal axis defined by the orientation of a distal tip of catheter sheath 10. This is due to a predefined bend in second device 601 which it assumes upon unsheathing. Such a bend might allow, for example, sideways access to a surface, and/or be a feature of a guidewire allowing selection of an off-axis aperture to advance into.

A physician monitoring the increase in radial offset optionally uses this to learn the direction of radial offset, and/or to help gauge if the device is expanding unimpeded, or if it is being impeded, for example, by contact with a lumenal wall instead of a targeted aperture.

System for Second Device Position Estimation

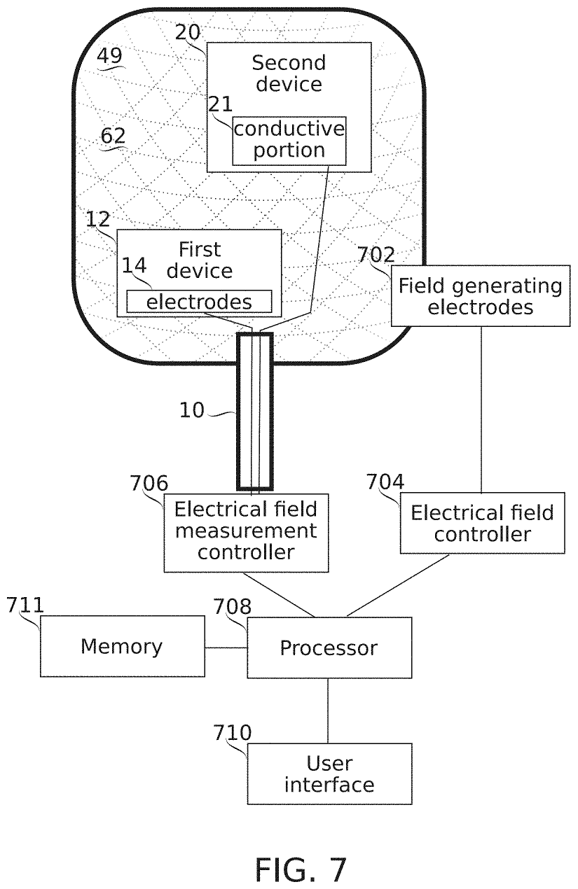

Reference is now made to FIG. 7, which schematically illustrates a system 700 for navigating a device within a body cavity 49 using electrical field measurements, according to some embodiments of the present disclosure. In some embodiments, the system of FIG. 7 is applicable, for example, to perform the method of any one or more of FIGS. 1A, 2, and/or 3.

In some embodiments, system 700 comprises elements in one, two or three of the following three categories, including at least the processing equipment category. The categories are:

Probes and Electrodes:

Probes may be adapted particularly for use with system 700, for example by modification to include electrodes. Probes and electrodes may be useful with system 700 as-is. In either case, system 700 is optionally provided along with any one or more of the probe and/or electrode devices next listed, e.g., as a kit, or separately (e.g., as accessories). Examples of such probes and electrodes include:
Catheter sheath 10, for example as described in relation to FIGS. 1B-1E,4A-4C, 5A-5C, and/or 6A-6C. In some embodiments, first device 12 and second device 20 are delivered through the same catheter sheath 10 (e.g., in alternation). In some embodiments, first device 12 and second device 20 are delivered through different catheter sheaths 10.
First device 12—which may be, more particularly, an electrode catheter insertable to a body cavity 49—comprising a plurality of measurement electrodes 14, for example as described in relation to FIGS. 1B-1C.
Second device 20, which is a device insertable to a body cavity 49; for example as described in relation to FIGS. 1D-1E, 4A-4C (second device 400), 5A-5C (second device 500), and/or 6A-6C (second device 600).
Electrical field generating electrodes 702, comprising a plurality of electrode which may be external (i.e., body surface) electrodes and/or electrodes insertable to a body to generate electrical fields (e.g., electrical fields 62, 62A, 62B, and/or 62C).

Electrical Field Generating and Recording Equipment:

Electrical field generating and recording equipment for use with system 700, may be standard equipment, or it may be adapted to it particularly, for example by modification to include additional channels, frequency generation ranges, and/or communication protocol capabilities. In either case, system 700 is optionally provided along with any one or more of the electrical field generating and recording devices next listed, e.g., as a kit, or separately (e.g., as accessories). Examples of such probes and electrodes include:
Electrical field controller 704, configurable to generate a plurality of electrical fields 62 (e.g., electrical fields 62A, 62B, 62C as illustrated in FIG. 1B) within a body cavity 49, wherein the electrical fields are optionally distinguished from each other by frequency, time of generation, and/or selection of electrode field generating electrodes 702 used to generate the electrical field. Optionally, a plurality of electrical field controllers combine to perform the functions of electrical field controller 704.
Electrical field measurement controller 706, configurable to receive electrical signals from an electrically conductive portion 21 of second device 20 and/or electrodes 14 of first device 12, optionally at the same time or alternately. In some embodiments, electrical field measurement controller comprises a voltmeter, ammeter, ohmmeter, impedance meter, or other electrical property measuring device. Optionally, a plurality of electrical field measurement controllers combine to perform the functions of electrical field measurement controller 706.

Processing Equipment:
Processor 708, configured (e.g., via access to instructions stored in memory 711) to receive to and/or access electrical field measurements from the first device 12 and/or the second device 20, perform processing operations including position estimation rule generation and application, position estimation rule calibration, and/or position estimation rule modification. Optionally, processor 708 exerts control over electrical field controller 704 and/or electrical field measurement controller 706, e.g. to set and/or select parameters.
Memory 711, comprising a computerized storage device configured with data and instructions that instruct processor 708 to perform functions described herein, according to one or more of the particular embodiments. In some embodiments, memory 711 is more particularly configured with calibration and/or configuration data appropriate to devices provided to system 700 from one or both of the other categories; for example, data which allow conversion of electrical measurement data into positions according to the probe and/or electrode type(s) (e.g., types of the first device 12 and/or second device 20) interconnected with electrical field measurement controller 706. In some embodiments, a computer storage medium (or media) is/are provided which contains the instructions and/or data for use by a processor 708, and the rest of system 700 is provided separately; e.g., assembled from components which are then configured to operate according to the instructions and data stored on the computer storage medium/media
User interface 710, optionally provided along with system 700 or separately provided; and used to display position estimation results and/or receive user input controlling operations of processor 708.
   Second Devices with a Plurality of Conductive Portions

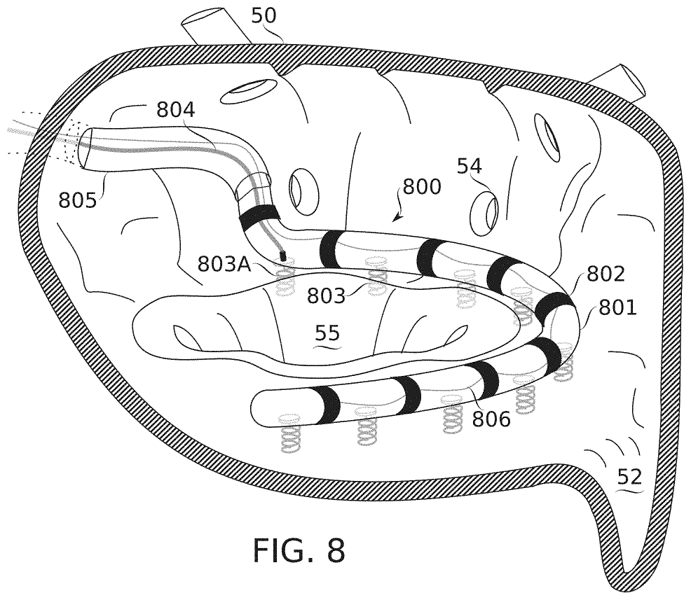

Reference is now made to FIG. 8, which schematically represents an implantable device 800 for modifying the circumference of a heart valve 55, and comprising a plurality of electrically conductive fasteners 803 used to secure implantable device 800 to the wall of a heart left atrium 50, according to some embodiments of the present disclosure. Heart left atrium 50 is shown in cross-section.

Implantable device 800, in some embodiments, comprises a flexible member 801, which is configured to be secured to tissue around the perimeter a heart valve 55 using fasteners 803 (optionally screws). Upon being secured, the device can be cinched, shortening flexible member 801 and drawing the perimeter tissue together. This potentially treats leakage/regurgitation through heart valve 55 (a heart mitral valve, in the example shown) by bringing leaflets of the valve closer together.

Implantable device 800 is shown during a late stage of implantation, with the flexible member 801 still partially within catheter sheath 805 (from which it has been partially extruded). Several fasteners 803 have already been inserted to tissue, and fastener 803A (also electrically conductive) is in the midst of being inserted. Insertion is performed, in some embodiments, by rotation of cable 804 to which fastener 803A is removably attached. Cable 804 is itself also electrically conductive, and in electrical contact with fastener 803A.

In some embodiments, each fastener 803 is brought separately from a proximal end of the catheter sheath 805 to be secured into place by mechanical operation of cable 804. In some embodiments, cable 804 is attached to an electrical field measuring device, thereby converting fastener 803 (e.g., fastener 803A) into an electrical field measuring electrode. In some embodiments, this allows fastener 803 to act as a second device, for example as described in relation to FIG. 1A. Optionally, manipulation of fastener 803 by cable 804 produces changing measurements during fastening. These measurements may indicate the position of fastener 803, for example as described in relation to FIG. 1A. Additionally or alternatively, information about the progress of implantation may be inferred from the measurements. For example, as more of fastener 803 embeds in tissue, it may acquire a higher impedance and/or a changed equivalent first-device electrode position. Optionally, this information is used to track progress of implantation.

Radio-opaque markers 802 are optionally comprised of a conductive material (e.g., a radio-opaque metal). Optionally one or more of radio-opaque markers 802 are connected to a conductive wire 806 which extends to a proximal side of catheter sheath 805, where it is optionally connected to an electrical field measuring device. Radio-opaque markers 802 are optionally connected to a single wire 806, or to a plurality of separately conducting wires 806. When separately wired, positions of individual radio-opaque markers are optionally determined according to their equivalent first-device electrode position. When wired to a same conductor in common, the relationship of the equivalent first-device electrode position to the positioning of the radio-opaque marker 802 is potentially offset, e.g., to near a geometrical “center of gravity” of the electrically joined-together radio-opaque markers 802, or another location.

Movements of the equivalent first-device electrode position during implantation may provide additional information about device status (which is optionally used to track progress of implantation). For example, as each new electrically joined-together radio-opaque marker 802 is exposed upon extrusion from catheter sheath 805, there is potentially a relatively rapid (e.g., step-function) jump in estimated position. As the radio-opaque marker 802 approaches the wall of the left atrium 50, there may be an additional rapid change in estimated position, e.g., insofar as field density lines may be more concentrated near the body cavity wall, so that the contribution of the radio-opaque maker 802 to the overall measurement changes more rapidly as a function of its change in position.

It is noted that one or more radio-opaque markers 802 wired as one or more electrically isolated electrodes and/or a fastener 803 electrically isolated from the radio-opaque markers 802, optionally together form an electrode system comprising a plurality of electrodes. By controlling distances of extrusion of flexible member 801 and fastener 803, the distances of these elements (considered as electrodes) is optionally controlled so that it is known, and this system, in some embodiments, then used as a plurality of electrodes for self-scaled mapping of the body cavity, for example, as described for a first device in the overview, and/or in relation to FIGS. 1A-1C, herein.

Also shown in FIG. 8 are left atrial appendage 52 and pulmonary vein roots 54.

### Planning/Presentation of Ballistic Sheath/Catheter Movements

Planning of Sheath/Catheter Movements

Reference is now made to FIG. 9A, which is a flowchart schematically representing a method of selecting a location 900 for transseptal crossing by a device delivery sheath 10B, according to some embodiments of the present disclosure. In some embodiments, the location 900 is selected so that further advance of the delivery sheath by pushing brings it to the position from which a device it delivers (delivered device) can be deployed and/or activated. By a “pushing” motion is meant motion induced along a path by a simple advance of the device, without use of additional actuations/commands for steering. Simple retraction along the same path is a pulling motion. Use of pushing/pulling motion to reach a target has potential advantages, for example to reduce a complexity and/or risk of a procedure. Pushing motion to actually reach a target begins from a suitable initial configuration. Optionally, another navigation element of the catheter is actuated as part of targeting, e.g., to adjust a steering angulation of the catheter. In some embodiments, predicted and/or simulated advance of the catheter is without steering, which is a potential advantage for simplicity, but this does not preclude use of steering in the actual advance, for example, to keep the forward motion on target. In some embodiments, steering angulation of the catheter is also predicted and/or simulated, accepting that this may make the overall motion more complex.

Reference is also made to FIG. 9B-9C, which schematically illustrate configurations of elements involved in the method of FIG. 9A, according to some embodiments of the present disclosure. In some embodiments, device delivery sheath 10B is a delivery sheath for a LAAO device, for example, LAAO device 21.

The method of FIGS. 9A-9C is described in relation to use of an LAAO device 21, targeted to an ostium of the LAA. However, it should be understood that the method is optionally adjusted to guide transseptal crossing (by puncture of the interatrial septum, optionally at or near the fossa ovalis) preliminary to motion across the left atrium by another device, and optionally to another target of the left atrium.

For example, in some embodiments, a transseptal crossing location for a sheath of a cryoablation catheter is selected to reach a root of a pulmonary vein, and/or a position from which the cryoablation catheter is readily steered to all or a selected plurality of pulmonary vein roots.

At block 902 (FIG. 9A), in some embodiments, measurements by a first device are accessed which provide a 3-D structural model of a left atrium of a heart. The measurements are optionally accessed indirectly as the 3-D structural model itself, as images from which the 3-D structural model is generated, and/or as electrical measurements from which a spatial representation of the space in which the measurements were taken is reconstructed. Herein, a “V2R” model refers particularly to a 3-D structural model, which models a structure in a spatial domain R, based on reconstruction from electrical field measurements obtained in an electrical field-measurement domain V (e.g., voltage, impedance, or another electrical field measurement).

In some embodiments, the first device is an intracardiac measurement device used for electrical field, ultrasound, and/or magnetic field-based mapping. In some embodiments, the first device comprises an electrode probe delivered by catheter to the heart, e.g., via a first transseptal crossing location. The electrode probe is optionally a multi-electrode probe, wherein the electrodes are positioned, for example, along a: straight line; closed or partially-open loop (e.g., lasso or circle); and/or a plurality of straight or curved segments arranged to approximate the outline of a sphere, hemisphere, umbrella spokes, or another shape.

The 3-D structural model comprises indications of at least the shape of the septal wall 53 from the left atrial side, the shape of an aperture (the LAA ostium) into the left atrial appendage (or another target, according to the device being used and/or procedure being performed), and the relative positions of each. In some embodiments, the 3-D structural model comprises indications of other features of the lumenal wall of the left atrium, for example, positions of the pulmonary veins and/or the mitral valve.

At block 1904, in some embodiments, a target region within the LAA ostium (for example) is defined. The defining optionally is performed manually (e.g., by means of an operator selecting a position on a 3-D display of the 3-D structural model), and/or automatically. In some embodiments, an operator can command the system (e.g., by pressing of a foot pedal, or by providing another input) to indicate an estimated shape and position of the LAA, e.g. (FIG. 9B), a representation 911 which approximates the shape of LAA 52, e.g., as a cylinder. Optionally, a preferred portion of the target region is marked by the operator on the estimated shape representation 911. In some embodiments, the defining comprises selection of a point 915 (and the target region comprises a region surrounding the point). In some embodiments, the defining comprises selection of a line segment (and the target region comprises a region surrounding the line segment, e.g., a cylindrical or ovoid-shaped region). Trajectory 910 represents a trajectory between a targeted crossing location 900 and a preferred portion of the target region 915.

Automatic determination, in some embodiments, comprises defining a geometrical center (“center of gravity”) of a 2-D cross-section of the 3-D structural model, wherein the 2-D cross-section includes a circumference of the LAA ostium. Determination of the LAA ostium in turn may comprise, for example, automated registration of the 3-D structural model to a predefined reference model of the LAA, selection of the LAA ostium using a user interface input from an operator, or another method.

Optionally, the target region (or a preferred portion thereof) is selected to be approximately centered within the LAA ostium and/or the walls of the LAA. Optionally, the target region (or a preferred portion thereof) is deliberately offset from a centered position; for example, to allow for a larger range of insertion depth of the delivery sheath 10B without puncturing, e.g., the LAA wall. This is a potential advantage when the LAA itself is curved and/or oriented at an angle significantly oblique to the direction between it and the septal wall 53.

The target region of the LAA optionally has a depth; e.g., a depth within about 1 cm of a preferred portion of the region targeted for deployment of the LAAO device. However, where such a depth is unavailable (e.g., because of curvature of the LAA), a shallower depth may be targeted.

As noted already, the target region is not necessarily a single point, even if initially defined (e.g., by the user) using a single point. For example, it can be defined as a region surrounding a point or one-dimensional selection, for example, expanded from the selection by some distance (e.g., 1-5 mm) in two or three dimensions. The target region, in some embodiments, comprises any position within the ostium circumference (optionally offset from the ostial wall, e.g., by a safety factor of 2 mm or more). This may be acceptable in view of the optional capability of an LAAO device (for example) to self-center upon expansion. Nevertheless, in some embodiments, there may be “more preferred” and “less preferred” portions within the target region, with the more preferred portion being what is actually targeted, and the less preferred portions being acceptable in case the more preferred target portion cannot be reached, entails a certain level of risk that it will not be reached as planned, and/or is in fact not reached.

At block 905, in some embodiments, estimated initial (that is, pre-crossing, within the right atrium) position and mechanical properties of delivery sheath 10B (a catheter) and/or the delivery sheath 10B with a device such as LAAO device 21 are accessed.

In some embodiments, the initial position of delivery sheath 10B is determined using measurements from one or more electrodes of delivery sheath 100 and/or a portion of the device it delivers (e.g. an LAAO device 21), and referencing those measurements to a V2R model which supplies the structural model. The position is optionally determined along a distal portion of the delivery sheath shaft; for example, it includes the position and orientation of a tip of the delivery sheath, and the position and orientation of a more proximal portion of the delivery sheath. Optionally, position and orientation in between these locations is determined by measurement and/or modeling, e.g., modeling based on constraints defined by the more distal and more proximal positions, optionally together with the use of mechanical stiffness/bending properties of the catheter sheath. Positions of any part of the delivery sheath 10B are optionally determined in part by use of geometrical constraints (e.g., it may be known that delivery sheath 10B enters the right atrium from the inferior vena cava).

The pre-crossing position of the delivery sheath 10B within the right atrium is defined relative to the septal wall 53 so that an orientation of its distal end may be calculated for each position on the septal wall 53 from which penetration into the left atrium may be initiated. The mechanical properties of the delivery sheath 10B optionally include its stiffness and/or range of steerable angulation (e.g., steering by actuation of a mechanism provided on the delivery catheter). Stiffness optionally varies with steering angulation direction and/or degree of bending. In some embodiments, a delivery sheath 10B is biased to bend in a particular direction with a certain force and/or radius of curvature, and the accessed mechanical properties includes estimated quantification of this bending bias. Optionally, the mechanical properties include variations in mechanical properties as a function of parameters such as the degree to which another tool (and optionally, for example, the LAAO device 21 itself) is advanced and/or actuated within sheath 10B.

In some embodiments, the initial position of the LAAO device delivery catheter is selected “in reverse”, based on initial positions that lead to a targeted outcome, e.g., reaching the target region of block 904 by an advancing motion after septal crossing. This can be done, for example, by simulating many different initial positions, and choosing one or more initial positions that lead to the targeted outcome. In some embodiments, these initial positions themselves become “targeted” for a user, for example by presentation in block 908; and the user is optionally prompted to make a measured configuration of the delivery catheter match the targeted configuration.

At block 906, in some embodiments, at least one transseptal crossing location is selected, based on the first-device measurements of block 902, the LAA ostium target region of block 904, and the position and mechanical properties of block 905. Optionally, the trans septal crossing location is selected as a range of crossing positions and/or crossing angles.

The position and mechanical properties of delivery sheath 10B potentially limit the range of positions at which the septal wall 53 can be crossed, and/or the range of crossing angles which can be achieved at each crossing point. From within these ranges, more preferred crossing points are optionally selected as those which allow the target region of block 904 (and optionally a preferred sub-region thereof) to be reached by advance of the delivery sheath across the left atrium from the crossing point; that is, a simple advance of the delivery sheath without a requirement for the adjustment of steering control. A simple advancing movement (that is, advance of the catheter performed without lateral steering adjustments) is preferred, in some embodiments, because it allows achievement of a confirmed crossing point configuration to be predictive of final position, optionally without a need for trial and error navigation to the LAA ostium. This in turn is a potential advantage since repeated probing in the region of the LAA may entail an elevated risk of damaging the heart wall, and/or of dislodging a blood clot into the circulation where it may cause an ischemic event.

At block 908, in some embodiments, a selected crossing location (and/or range of selected crossing locations) is indicated to the operator. Optionally a more complete starting configuration of the delivery sheath 10B is indicated to the user (e.g., as an image), which also details an approach configuration to the selected crossing location, e.g., by shape, and/or in the form of descriptions of how to adjust the catheter sheath 10B.

The indication optionally comprises an indication on a 3-D image of the heart which shows one or more locations and/or orientations (e.g., in the vicinity of the fossa ovalis) from which an advancing motion to the LAA ostium is calculated to be successful. Optionally, the crossing position is indicated together with other aspects of the configuration of the delivery sheath, e.g., a particular orientation and/or steering angulation of the delivery sheath at the crossing position. Optionally, a single crossing position is associated with only a single set of other crossing configuration parameters. Optionally, a crossing position is reached from a plurality of different crossing configuration parameters, e.g., if the delivery sheath is a steerable delivery sheath.

Optionally, all crossing positions and/or crossing configurations leading to the targeted location are considered available to the operator. For example, as the operator adjusts the sheath in preparation for crossing, the assumption of any starting position of the sheath (e.g., its tip, or an extended region of the sheath) which is within the acceptable range for target-reaching advancing motion is responded to by an indication (e.g., tone, color, text, vibration, or other sensory indication) of this condition. Sensing of crossing position is further described, for example, in relation to block 924 of FIG. 9D. Optionally, a range of acceptable crossing positions is indicated on a 3-D image comprising a display of the septal wall 53.

Where there is considered to be a certain degree of targeting risk (e.g., due to uncertainty in relative positions, movements due to heartbeat, or another risk), the allowable crossing positions and/or crossing configurations of the delivery sheath 10B are optionally reduced to those where the tolerance range remains acceptably within and/or close to the position of the target location. Optionally, such crossing positions/configurations are allowed, but indicated as less preferable.

In some embodiments, the ranges of crossing positions and/or crossing configurations (e.g., ranges of delivery sheath angulation at crossing) are further refined by a weighted evaluation function, optionally implemented by computer processor instructions. The evaluation function optionally takes into account parameters affecting likely success of the crossing itself (e.g., a crossing which is more nearly perpendicular to the septal wall 53 and/or uses the sheath in a stiffer configuration is potentially more likely to succeed). Optionally, the evaluation takes into account predicted accuracy of targeting; e.g., a crossing which will allow advancing motion to a more preferred region of the target is weighted higher than a crossing which will reach a less preferred region of the target. Crossing configurations which are potentially more confounded by risk (e.g., due to motion, position uncertainty, or another factor) are optionally weighted as less preferable.

It is noted that the target and/or a preferred sub-region of the target may itself vary somewhat as a function of crossing position and/or crossing configuration; for example, according to how close the catheter tip could come to a wall of the LAA during advancement, and/or if advanced more than planned. Additionally or alternatively, these factors are included in the weighting evaluation.

In some embodiments, e.g., if it appears that no crossing position/configuration allows a simple advancing motion that will effectively and/or safely reach the target location, a range of crossing positions/configurations is indicated to the user which can reach the target with actuation of a catheter's steering mechanism. Optionally, these are weighted according to an estimate of how much additional steering is required to reach the target location, e.g., according to how much position change is needed, and/or how complex the steering command is (for example, if the steering command is variable in amplitude, and/or requires two or more separate inputs). Optionally, the indication of these regions includes indications of suggested steering inputs to adjust the otherwise advancing motion. Potentially, using these indications as guidance increases a likelihood of reaching the target location without error.

FIG. 9B illustrates a pre-crossing configuration, wherein delivery sheath 10B is being moved about within a right-atrium side of the atrial septal wall 53, subsequent to mapping left atrium 50 using electrodes 14 of electrode probe 12 of mapping catheter 10.

Prediction of Ballistic Sheath/Catheter Movements

Reference is now made to FIG. 9D, which is a flowchart schematically representing a method of calculating an advancing trajectory of a catheter sheath 10B, according to some embodiments of the present disclosure.

Additionally or alternatively to the method of FIG. 9A, a calculated advancing path of a delivery sheath (all the way to about the position of the target region in the left atrium) is shown at each new position and/or configuration of the delivery sheath. The operator thus may optionally determine for themselves whether a simple advance from the current position or a steering-adjusted advance from the current configuration is likely to be successful.

At block 922 (FIG. 9D), in some embodiments, first-device measurements are accessed, for example as described also in relation to block 902 of FIG. 9A.

At block 924, in some embodiments, a transseptal crossing position of a delivery sheath 10B is accessed—that is, a position of delivery sheath 10B as it is (e.g., estimated to be from data), will be (e.g., is planned to be), or could be (e.g., as an available planning option) in preparation for making a transseptal crossing into the left atrium. The transseptal crossing position, in some embodiments, is determined from information available that describes a current position and/or configuration of the delivery sheath.

This information optionally includes a current state of the delivery sheath with respect to steering actuation, advancement into the right atrium, approach direction, and/or rotation (this information is optionally provided using one or more encoders configured to measure positioning and/or movements of the delivery sheath). The information optionally includes a measured position of the delivery sheath, e.g., based on X-ray and/or ultrasound imaging. Additionally or alternatively, the measured position of the delivery sheath comprises determination of a position based on measurements of electrical fields and/or magnetic fields sensed by an electrode or magnetic sensor co-located with (e.g., mounted to) the delivery sheath. In some embodiments, the current state of the delivery sheath is determined at least in part using mechanical properties of the delivery sheath and/or delivered device accessed at block 925, for example as described in relation to block 905 of FIG. 9A.

At block 925, in some embodiments, mechanical properties of the delivery sheath 10B and/or delivered device are accessed, for example as described in relation to block 905.

At block 926, in some embodiments, the trajectory is calculated. The trajectory calculation is a portion of the information described as being calculated in relation to block 906 of FIG. 9A.

At block 928, in some embodiments, the trajectory is presented, e.g., as part of a 3-D or 2-D image that indicates relative positions of the transseptal crossing position and other structures of the heart, such as an ostium of the LAA. Optionally, the trajectory is presented along with a target region; defined, for example, as described in relation to block 904. Optionally, an indication of a likelihood to reach or not reach the target region is provided. For example, the target region may not be reached if the delivery sheath is currently positioned to penetrate the interatrial septum at a wrong trajectory, and/or is too short to reach the target region.

Optionally, trajectory predictions are updated to match position information available for the delivery sheath even after it has crossed the septum, still taking into account, e.g., the estimated crossing position and other inputs described such as mechanical properties of the delivery sheath.

Device delivery sheaths have particular reference to device delivery sheaths, as these tend to have well-defined targets, as opposed, e.g., to mapping catheter probes which are anyway scanned through a range of positions. However, it should be understood that the methods and descriptions relating to FIGS. 9A-9D optionally apply to intracardiac catheters other than device delivery sheaths. For example, the trajectory of a mapping catheter is optionally useful as a reference, e.g., to avoid inserting the catheter to a place such as an aperture of an LAA, where it might accidentally dislodge a blood clot.

Trajectory presentations are not necessarily shown only for post-crossing navigation; in some embodiment, trajectories are presented pre-penetration, e.g., to assist in targeting of the fossa itself.

### Example of Presentation of Pre-Crossing Trajectories

Reference is now made to FIG. 13C, which represents a two-angle view of a 3-D scene related to guidance of septal wall penetration, according to some embodiments of the present disclosure. A two-angle view is optionally used for one or both of planning and prediction of movements of a catheter sheath 10. FIG. 13C in particular shows a planning example for positioning of a catheter in preparation for transseptal penetration of the fossa.

In some embodiments, trajectories 1330 indicate alternative available trajectories for crossing at or near a fossa 1313, e.g., in regions 900A, 900B, 900C, and/or 900D. View 1338 is an external perspective view, showing relationships of the trajectories 1330 to portions of the heart including inferior vena cava 57 and septal wall 53. View 1310 shows a face view of septal wall 53. The inset overview images 1320 indicate the orientation of the heart shown in the respective associated view.

As illustrated, a plurality of trajectories 1330 are shown simultaneously, which potentially helps an operator decide which configuration of a catheter sheath 10 to select/cause it to assume. Optionally, just one trajectory is shown at a time, e.g., the trajectory which corresponds to the current configuration of the catheter sheath 10. The two angle view of FIG. 13C is useful in particular for illustrating overall conditions within the region of navigation, focusing on relevant structures (view 1338), and to clearly indicate selected (or optionally selected) target areas for crossing (view 1310).

Compared to the four-image view of FIGS. 13A-13B, the transverse and sagittal views are omitted, since, e.g., the target of transseptal crossing is flush to a surface (of the septal wall), so that there is no perspective parallax to contend with. In some embodiments, switching between different arrangements of views (e.g., between the two-view arrangement of FIG. 13C and the four-view arrangements of FIGS. 13A-13B) is performed automatically according to the requirements of the next procedure action. For example, if the next action is reaching a surface target (e.g., a fossa of an interatrial septum), the two-view arrangement is automatically selected; if the next action is reaching a target offset in space from a surface (e.g., a center of a LAA ostium), a four-view arrangement is optionally selected.

In some embodiments, accordingly, there are provided first and second display configurations: a first, two-view configuration like that of FIG. 13C, and a second, four-view configuration like that of one of FIGS. 13A-13B. The two- and four-view displays are updated to reflect changes in the position of the catheter as new data is acquired, allowing them to be used to guide procedure actions which control positioning of the catheter.

The first, two-view display configuration comprises a face-on view 1338 of a surface (e.g., right side of FIG. 13C), and an overview image view 1310 that shows a device such as a catheter from the side, so that a path of its travel toward a target on the face-on viewed surface is visible. The first, two-view display configuration is potentially of particular use in particular for approaching, e.g., a substantially flat target. It may optionally show an indication of the catheter itself, or another indication, e.g., of an area which the catheter is approaching.

The second, four-view display configuration adds to these two views (represented in inn FIGS. 13A-13B by views 1304 and 1308) two mutually orthogonal lateral-side image views 1302, 1306, which show the catheter in an estimated position relative to tissue near it. This display configuration is potentially useful, in particular for approaching and/or interacting with a target that curves to at least partially surround a catheter distal tip and/or tool deployed from the catheter distal tip.

The first display configuration is shown, e.g., for use during transseptal crossing of an interatrial septum, and the second display configuration is shown, e.g., during navigation of the catheter to a position with the left atrium, for example, a left atrial appendage.

In some embodiments a computer processor which is instructed by instructions stored in a computer memory (e.g., a storage medium) is configured thereby to switch between the first and second display configurations according to the situations described. The switch may occur upon an explicit selection of a user interface option, and/or it may occur automatically upon receipt of a suitable input signal, e.g., as a result of detecting that tools being used have been switched to a device designated for use in the left atrium (as opposed to use for transseptal crossing as such), upon detection that positioning data indicates that a second atrial lumen has been entered after first exploring a first atrial lumen, and/or upon receiving and interpreting any other explicit or heuristic signal associated with the crossing which may be provided.

### Guidance to Avoid Aortic Puncture During Transseptal Crossing

Reference is now made to FIG. 9E, which is a flowchart schematically representing a method of estimating transseptal crossing positions posing an elevated risk of aortic penetration, according to some embodiments of the present disclosure.

A potential complication of an atrial septal crossing is accidental damage to the aorta, e.g., by penetration. The risk arises because a portion of the aorta passes between the right and left atrium, adjacent to the interatrial septum. Due, for example, to mispositioning and/or anatomical variation, it is possible that a candidate crossing position is too close to the aorta for safety. The aorta undergoes pulsatile motions, and this pulsatility has a characteristic phase which may be used, in some embodiments, to classify the candidate crossing position as endangering or non-endangering of the aorta.

At block 1202, in some embodiments, a catheter with an electrode on it and/or an electrode-equipped device delivered through the catheter is brought into a candidate crossing position (i.e., a position believed to be at the atrial septal wall).

At block 1204, in some embodiments, indications of capacitance (e.g., capacitance as a contributing component of overall impedance) are measured using the electrode. Capacitance contributions need not be isolated, e.g., from a resistance of the impedance measurements; the relevant portion of the signal is its time varying value as a function of heartbeat movements. The capacitance potentially varies over the course of one or more heartbeat cycles, e.g., due to movements of the heart causing changes in the proximity of the heart wall to the measurement electrode. In some embodiments, the measurements are synchronized to a measure of heartbeat phase, e.g., a body surface ECG. In some embodiments, measurements made from several candidate crossing positions are compared with each other to align them each to a same phase of heartbeat motion. In some embodiments, measurements are aligned to a characteristic phasic feature of the measurement timecourse.

At block 1206, in some embodiments, the timecourse of the measurements from the electrode are classified (e.g., by calculations performed using a computer processor) as indicating an aortic-like pattern of pulsatility, or not. In some embodiments “aortic-like” means that the measurements include a signal component which is the opposite “fossa-like” (septal wall-like) pulsatility. More particularly, the septal wall-like signal component tends to include pulsations synchronized to the time of atrial contraction, while the aortic-like signal component teds to pulse synchronized with ventricular contraction. Optionally, both types of signal component occur together in measurements from some positions.

For example, the aortic-like pattern includes a signal component due to pulsatile bulging of the aorta during ventricular contraction. The signal component optionally comprises, for example, a transient rise in capacitance as aortic bulging presses the heart wall toward the electrode. However, irrespective of the polarity of the change, the phase timing of the aortic transient relative to the time of atrial contraction is itself indicative. Where this transient is detected (optionally, detected above a threshold value, e.g., above about 20% of the peak amplitude detected in any location), the candidate crossing position is optionally considered aorta-endangering.

At block 1208, in some embodiments, the capacity classification is presented. Optionally, the presentation is in the form of a warning against proceeding with crossing, or another signal that indicates aortic proximity. Additionally or alternatively, in some embodiments, the crossing position is indicated on a 3-D representation of the heart, along with display coding (e.g., color coding, optionally in a warning color such as red) that indicates aortic proximity. The operator is then able to correct to move away from the aorta.

In some embodiments, the measurement electrode is moved over a portion of the septal wall with classifications performed at several locations within the portion to generate a map of aortic endangering and non-endangering candidate crossing positions. Optionally, selection of a final crossing position is delayed until a region having risky aortic proximity is characterized, as a way of providing additional confidence that the crossing position will not damage the aorta. Optionally, the final crossing position is selected to avoid the region of risky aortic proximity by a further safety margin, e.g., a margin of at least 5 mm, or another distance.

### Verification of Device Deployment

Deployment Verification Using Closure-Induced Impedance Changes

Reference is now made to FIG. 10A, which is a flowchart schematically representing a method of verifying implant positioning, according to some embodiments of the present disclosure. Reference is also made to FIGS. 10B-10C, which are schematic graphs of impedance over time for stable and unstable closure by an LAAO device, according to some embodiments of the present disclosure. Reference is also made to FIG. 10D, which schematically represents an electrical measurement configuration related to the measurements of FIGS. 10B-10C, according to some embodiments of the present disclosure.

FIG. 10D illustrates elements of the electrical configuration underlying the method/measurements of FIGS. 10A-10C. During closure by an LAAO device 21, two blood pools 1001A (e.g., inside the LAA 52), and 1001B (e.g., outside the LAA 52) become relatively electrically isolated from each other due to the interposition of metallic device 21 and the relatively insulating membrane 1004 (e.g., a fabric membrane) attached thereto. Device 1003 is attached to an electrical impedance measuring device, allowing an impedance 1005 to be measured, e.g., relative to the voltage of an electrode 1007 configured for use as a ground reference (e.g., an electrode attached to a leg body surface).

At block 1032 (FIG. 10A), in some embodiments, an occlusive device is deployed (e.g., LAAO device 21 is expanded to about the configuration shown in FIG. 10D). Measurement may be performed using the occlusive device itself as an electrode. Additionally or alternatively, in some embodiments, measurement is performed using body surface electrodes; the occluder device potentially modifies electrical paths between body surface electrodes differentially, based on whether it fully occludes the LAA, or only partially (and, more particularly, modifies the paths for impedance measured, e.g., at RF frequencies).

As the occlusive device expands, impedance is measured through an electrically conductive portion of the device at block 1034. Increasing size (e.g., increasing exposure of surface area of the occlusive device and/or volume through which the occlusive device extends), leads to a drop in impedance 1012, 1022 (FIGS. 10B, 10C) measured using the device itself as an electrode.

Additionally or alternatively, in some embodiments, impedance changes are measured through body surface electrodes. Further expansion begins to create a closure, cutting off conductive pathways through blood, eventually resulting in a rebound in impedance, e.g., as shown during time periods 1014 and 1024. Contact with the heart wall itself may also contribute a component of impedance change. If the rebound 1014 is missing or attenuated in amplitude, it potentially indicates (e.g., is classified as indicating) that deployment seriously failed, i.e., it apparently has not taken place within a closely confined space as expected.

As expansion continues, closure is potentially formed, e.g., during period 1015. Potentially after interruption by one or more periods of reconfiguration/settling 1016 having relatively noisy impedance (e.g., a period with noise amplitude at least 2×, 4×, 5×, or 10× the amplitude of the background measurement noise), the impedance value settles to a stable value 1017, which provides an indication of apparently stable deployment.

In an alternative scenario, the period of post-expansion noise in the impedance 1026 does not settle to a quiet state, providing an indication that deployment may not be well-closing and/or stably anchored.

In some embodiments, observation of the timecourse of impedance (e.g., as displayed on a screen) is used to qualitatively estimate deployment status.

Additionally or alternatively: at optional block 1036, in some embodiments, a processor configured to analyze impedance operates to classify impedance measurements as they are taken. The processor is optionally configured to distinguish between and identify any two or more of the states described in relation to block 1034. For example:
A period 1012, 1022 of rapid impedance decline down to a minimum is identified as a period of device expansion.
A period 1014, 1024 of impedance rebound is identified as indicating that the device is expanding within the confines of an aperture of about the dimension of the device.
A subsequent period of unstable impedance 1016, 1026, or of only brief stability 1015 (e.g., stable for less than a heartbeat cycle, less than five seconds, or less than another brief period) is identified as indicating that device anchoring is itself unstable and/or incompletely closing. Periods of instability may be further characterized by correlation with the heartbeat phase; for example, device anchoring may loosen during a certain phase of heart contraction, at which noise occurs.
A period of longer stability 1017 (e.g., stable for at least several second, e.g., 5 or more seconds) is identified as indicating that the device anchoring is adequately stable and/or closing. Optionally, the device is deliberately wiggled and/or tugged at in order to test that it is firmly seated; partial loosening which results potentially interrupts the period of stability 1017 with a return to instability.

At optional block 1038, in some embodiments, the identification is presented as guidance for an operator. The guidance optionally comprises an indication which converts one or more of the features automatically identified in the impedance timecourse to an indication of device status; e.g., an indication corresponding to one or more of the identifications described in relation to periods 1012, 1022, 1014, 1024, 1015, and 1017.

In case that the occlusion device 21 fails to achieve a targeted level and/or stability of closure, the operator optionally takes a corrective action. For example, the operation may re-collapsing device 21 and attempt deployment again (e.g., at a slightly different position).

Optionally, a current phase of device 21 opening is estimated using the impedance drop phase 1012, 1021; and displayed, for example, as described in relation to FIG. 12A.

Dielectric Dilution-Based Deployment Verification

Reference is now made to FIG. 11A, which is a flowchart schematically representing a to method of verifying surgical closure of an opening between two fluid-filled compartments of a body lumen, according to some embodiments of the present disclosure. Reference is also made to FIG. 11B, which shows example time courses of the movement of an injected iodine solution across leaking and closed deployments of an LAAO device, according to some embodiments of the present disclosure. Further reference is made to FIG. 11C, which shows example time courses of the movement of an injected saline solution across leaking and closed deployments of an LAAO device, according to some embodiments of the present disclosure.

In some embodiments, a correctly deployed implant device is a closure device that creates a characteristic level of closure that slows (e.g., the closure creates a porous “seal”) or prevents (e.g., the seal is non-porous) the exchange of fluid across it (that is, it affects the redistribution of fluid). A left atrial appendage occluder is an example of such a device. Examples of other such devices in the heart include devices for closure of an atrial septal defect, a patent (open) foramen ovalis, and/or a ventricular septal defect. It should be noted that a body lumen, for purposes of the descriptions of FIG. 11A, includes any two compartments in fluid communication with each other; for example, a left and right atrium together may comprise a “lumen”, insofar as they are interconnected by a patent foramen ovalis.

Additionally or alternatively, closure comprises use of a method other than implantation of a closure device; for example, glue and/or suturing.

In some embodiments, closure closes an opening in another organ, for example, an organ of the gastrointestinal tract (e.g., the stomach, for example to form a gastric sleeve).

At block 1102, in some embodiments, a dielectric contrast agent is injected into one of the two fluid-filled compartments. A dielectric contrast agent comprises, for example: hypertonic saline, ice cold saline, hypotonic saline, warm saline, iodine solution, or another solution with dielectric properties distinct from the dielectric properties of the fluid (e.g., blood) that normally fills the compartments. In some embodiments, the contrast agent is injected as a bolus. In some embodiments, the contrast agent is injected continuously or nearly continuously.

In some embodiments, the injected dielectric contrast agent has a higher or lower viscosity than the normal-filling fluid. For example, a solution iodine contrast agent (e.g., comprising about 320 mg I/ml) may have a viscosity (e.g., at 20°) of about 26.6 mm²/sec, compared to about 1 mm²/sec for water/saline. A range of typical viscosities of blood (at about 37°) is, for example, about 2.8-3.8 mm²/sec. Use of a higher-viscosity material potentially slows or prevents contrast agent migration, potentially making it easier to distinguish between faster and slower washout/dilution times.

Either of the two fluid-filled compartments are optionally used, in any combination of one or both compartments, for injecting from, injecting to, and measuring contrast agent in.

In some embodiments, injection from one compartment to the other (in particular) is achieved by using relative high pressure injection (e.g., across a porous membrane of the closure device), needle injection (e.g., using a needle inserted across the closure device from one chamber to the other), and/or port injection (e.g., using a port of the closure device, optionally a one-way valved port). In the case of an LAAO device, for example, the delivery catheter is optionally pressed against a fabric or other porous membrane of the deployed closure device from the side of the atrium, and contrast agent forced across the membrane. Contrast material injected under pressure across a membrane, once across the membrane, may return to the other side primarily by a slower diffusion mechanism (unless there is a sealing leak that allows return flow, e.g., by passing around the LAAO device). The amount of injected contrast agent fluid is, for example, about 10-20 ml. The injection is optionally continuous, or timed to coincide with phases of the heartbeat cycle, e.g., to a first 50%, a final 50%, and/or portions of either the first or final 50% of the heartbeat cycle. Optionally, separate injections are pulsatile with a repetition cycle that is longer than the cardiac cycle

At block 1104, in some embodiments, a timecourse of dielectric property changes is measured over a period of, e.g., the 5-60 seconds after a bolus of dielectric contrast agent is delivered. Dielectric change is optionally measured as a period of redistribution of the contrast agent: for example, dilution of the contrast agent within the compartment to which it was injected, and/or as a period of wash-in of contrast agent to the opposite chamber. In the timecourse data of FIGS. 11B-11C, the onset 1110 of contrast agent injection is followed by a rise (iodine, FIG. 11B) or fall (saline, FIG. 11C) in impedance which ends when injection ends. Afterward, impedance returns with greater or lesser speed towards its baseline value. In the raw (light-colored) data, High-frequency spikes correspond to changes in impedance (used as an indication of local dielectric properties) due to heart movements, one spike per heartbeat. The spikes can be filtered out (darker-colored), emphasizing the long-term trend. For analysis, the data are optionally fit by a decaying exponential, for example of the form:

$$\frac{{\%}_{c l e a r e d}}{beat} = 100 ( 1 - {e}^{- \frac{H . R .}{{k}_{2}}} )$$

Where H.R. is the heart rate, and k₂ corresponds to a time constant with which impedance returns to baseline. Closure corresponds to a value of k₂ larger than about, for example, 10 seconds, or larger than about 20 seconds.

Brief reference is now made to FIG. 11D, which illustrates closed vs. leaking timecourse measurements for a population of LAAO closure trials (in pig heart), according to some embodiments of the present disclosure

Clearance of a percentage of remaining contrast agent per beat

$$\frac{{\%}_{clea r e d}}{beat}$$

is a way of expressing the rate of contrast agent redistribution after injection. 109 trials were performed in total (divided among the different conditions). The ranges of population quartiles are shown by the dotted bars (top and bottom quartiles) and boxes (middle two quartiles). Outlier examples (beyond the nominal top quartile) are shown as + marks. A clearance time of below about 10 seconds (with either iodine or saline) also corresponded to a leaking device deployment. Iodine clearance was slightly slower than saline clearance.

With continuing reference to FIG. 11A: additionally or alternatively, in some embodiments, dielectric contrast agent is delivered steadily (e.g., in several small amounts optionally timed to the heartbeat cycle and/or continuously). The steady delivery potentially results in an increasing dielectric change (in the receiving chamber) once closure is achieved, while before that the dielectric change is prevented from increasing as much by dilution of the dielectric contrast agent into a larger pool of surrounding fluid. Additionally or alternatively, dielectric change in a non-receiving chamber is reduced and/or delayed as closure improves, due to less dielectric contrast agent passing into it, and/or passing in less quickly.

Detection of dielectric change is performed using an electrode positioned in the detection chamber (which can be either or both of the injected-to chamber, or a chamber which is potentially leaked-to after injection). The reference electrode is optionally, for example, another intra-body electrode (optionally, one located within the detection chamber, or on an opposite side of the closure device), or a body surface electrode.

In some embodiments (e.g., embodiments where the closure device is an LAAO device), the electrode is optionally an electrode of a sheath used to position and deploy the LAAO device, an electrode mounted on the LAAO device, and/or a portion of the LAAO device itself, configured or use as an electrode. Optionally, the reference electrode is another electrode selected from among any of these electrode types.

At block 1106, in some embodiments, the timecourse of dielectric property changes is optionally classified. The classification, in some embodiments, is performed automatically, e.g., by a suitably programmed computer processor. In some embodiments, the classification is between two categories: “leaky” and “non-leaky” (and optionally on a scale between the two). The criteria of classification are selected depending on the type of leak which is to be detected; e.g., some leaking is potentially permissible through and/or around an implant, as long as the rate of leakage is maintained below some threshold. For example an LAAO device, in some embodiments, comprises a fabric or other porous membrane that allows contrast agent to pass through it, but at a rate which is distinguishably slower than leakage through tissue-device gaps at a rate great enough to be of concern.

In some embodiments, for example, a well-closed LAAO device creates an at least partial seal wherein after an initial impedance change from baseline to its maximum amplitude of impedance change (post-injection), return to a baseline level (optionally, a level near baseline within 10% of the maximum amplitude) takes about 40 seconds or more. For a poorly-closed LAAO device, the return to baseline or near-baseline takes, for example, less than half that time, e.g., less than about 20 seconds, or less than about 10 seconds. Optionally, a certain time to return to baseline (e.g., 20 seconds) is selected as a threshold cutoff between “leaky” and “non-leaky” deployments. Optionally, a return to baseline/near baseline faster than some lower threshold (e.g., 10 seconds) is classified simply “leaky”; a return to baseline/near baseline slower than some higher threshold (e.g., 40 seconds is classified simply “non-leaky” and anything in-between is classified as intermediately leaky, optionally according to how close it is to the leaky or non-leaky threshold.

In some embodiments, dilution and/or leakage of the contrast agent (as measured by impedance) is classified by comparison to a baseline established by a model of how fluid crosses the closure device, how fluid is otherwise exchanged within the chambers (e.g., exchange through natural or artificial perfusion) and/or by example measurements obtained in tests of the device.

In some embodiments, the baseline model includes calculation based on an anticipated equilibrium or near-equilibrium distribution of dielectric contrast agent. For example, in some embodiments, dielectric contrast agent is injected into one of two compartments, neither of which is actively perfused. In such embodiments, the baseline is based on a distribution of the dielectric contrast agent in, e.g., equal concentrations in either compartment. Additionally or alternatively, a target level of contrast agent concentration to be maintained in event of successful closure is modeled, e.g., a concentration twice that expected in the case of failed closure, when the closure is expected to cut the combined volume of the two compartments in half.

In some embodiments, the compartments are perfused relatively slowly compared to the rate of mixing/diffusion, and the model models concentration as the result of a dynamic balance of perfusion and of fluid exchange between compartments.

In this example, the impedance change is measured from within the LAA, that is, on the side to which contrast agent is injected. The measuring electrode is optionally the LAAO device itself. It is noted that the measuring electrode need not measure only from the contrast agent-receiving chamber (the LAA), since as the contrast agent leaks across it, the pumping action of the heart quickly dilutes and/or clears the leaking contrast agent.

However, an electrode located on the “leaked to” side (e.g., the atrium) nonetheless is potentially capable of detecting the inverse signal. In the heart, for example, there may be a transient signal occurring with the frequency of the heart rate, as fluid is ejected from the LAA and/or accumulates in the left atrium during one period of the heartbeat, and is diluted and/or purged from the atrium during another period of the heartbeat. The signal may be larger and/or faster when there is a leak; the signal may be weak or non-existent when there is no significant leak. Optionally, a plurality of electrodes are placed in the vicinity of the device-closed aperture (for example, distributed along a straight, circular, or lasso-shaped electrode probe). Each such electrode potentially detects a change in impedance (e.g., a transient change) influenced by its relative proximity to the leak. In some embodiments, this is used to increase sensitivity of leak detection, and/or to help localize leaks to positions nearest electrodes that detect the largest and/or fastest change (optionally, an estimate of leak position is part of the leak classification). It should be understood that reverse-filling is optionally detected additionally or alternatively by a plurality of electrodes: for example, electrodes within the contrast agent-filled chamber detect inflow of the non-contrast (normal) fluid (e.g., blood) in bursts of higher amplitude and/or faster time course when they are positioned nearer to the leak that allows the inflow.

At block 1108, in some embodiments, the impedance classification is presented (e.g., displayed on a computer display) to an operator. The presentation optionally comprises presentation of a binary classification (e.g., leaky/non-leaky) and/or a scaled classification (e.g., a degree of a severity of leakage between leaky and non-leaky). The presentation optionally comprises an estimate of leak position. Optionally, classification is skipped; for example, the raw impedance time course of an electrode is presented, with interpretation left to the experience of an operator. Presentation, in some embodiments, uses the user interface of a computer, and takes the form of, e.g., an image, text, color (e.g., warning color), sound, or other sensory stimulus.

### LAAO Device Implantation Overview

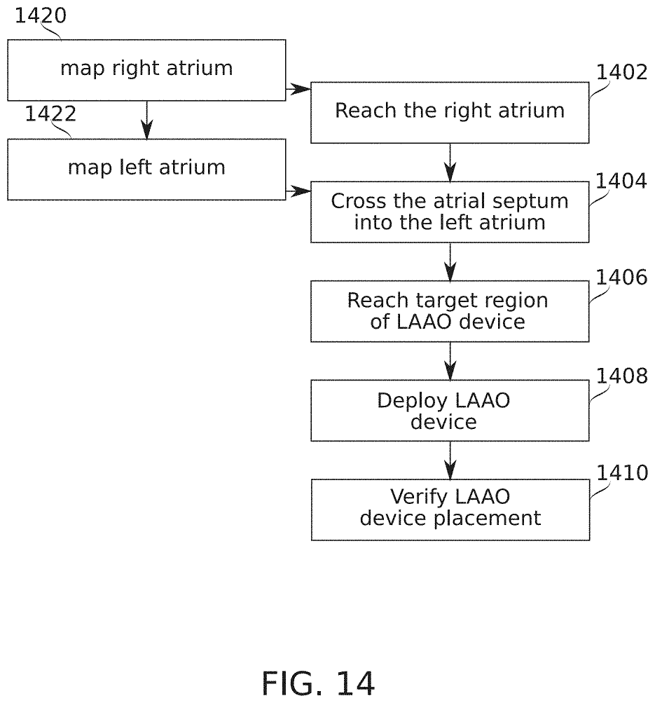

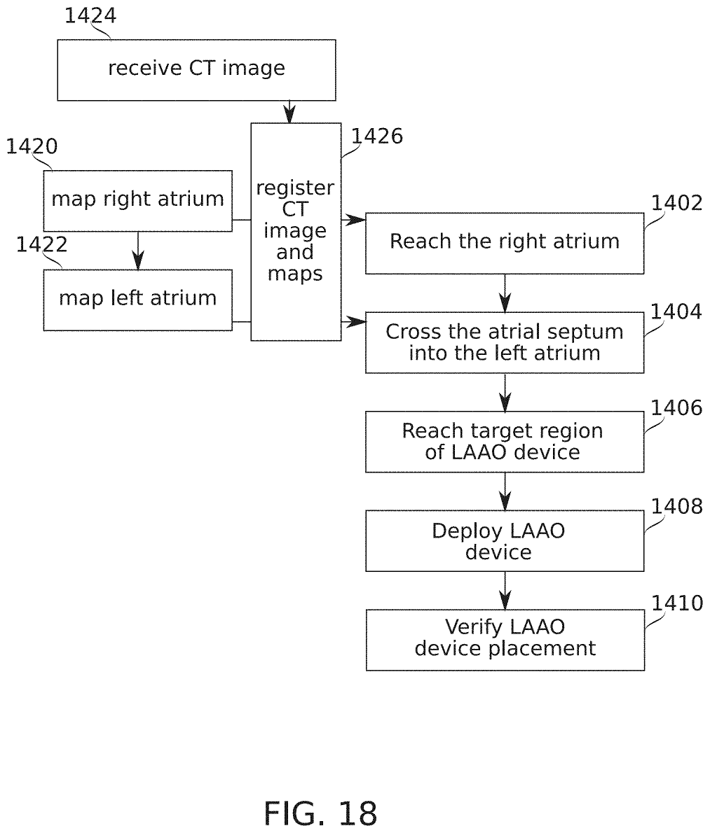

Reference is now made to FIGS. 14 and 18, which each show an overview flowchart of a method of deploying an LAAO (left atrial appendage occlusion) device, according to some embodiments of the present disclosure. A system used for carrying out the methods of FIGS. 14 and 18 optionally corresponds to the system of FIG. 7. Blocks of FIGS. 14 and 18 are the same, apart from the addition in FIG. 18 of blocks 1424 and 1426, which in some embodiments may affect particular implementation details of remaining blocks.

Blocks 1420, 1422 represent an optional method of obtaining heart structural data, comprising mapping the right atrium at block 1420, and mapping the left atrium at block 1422. Mapping is performed, e.g., by use of an electrode probe mapping distributions of electrical field potentials throughout the mapped chambers. Optionally, a same electrode probe is used to first map the right atrium, and then (after septal crossing) the left atrium. It should be understood that blocks 1402-1410 are optionally performed using structural data from other sources of structural information, for example, magnetic positioning data, ultrasound imaging, and/or X-ray imaging. In some embodiments (e.g., as illustrated in FIG. 18), structural information is augmented by receiving a CT image (block 1424), and, at block 1426, registering the maps of blocks 1420 and/or 1422 to the CT image. Examples related to CT image use are described in relation to FIGS. 17A-17C, herein.

Blocks 1402-1410 relate in particular to actions involving of a delivery sheath (catheter) which delivers the LAAO device into the LAA, and/or the LAAO device itself. The minimally invasive delivery approach shown is from the right atrium (e.g., from the inferior or superior vena cava) across the interatrial septum, and from there to the LAA itself. Embodiments of the present disclosure are not limited to this delivery approach, however, as an example, this approach allows placing several of the methods described herein within a larger context.

At block 1402, in some embodiments, the device delivery sheath reaches the right atrium. The structure of the right atrium is mapped out (e.g., at block 1420) fully or partially. Structural mapping includes, in particular, structures relevant to navigation of the device delivery sheath, such as the inferior vena cava and interatrial septum. Optionally, the position of the device delivery sheath is tracked using electrical field measurements made using one or more electrodes on the device delivery sheath, or using the LAAO device itself (partially advanced from the sheath, e.g., to a “mimicking” configuration) as an electrode, for example as described, e.g., in relation to block 105 of FIG. 1A. Optionally, the position of the device delivery sheath is estimated using information about its mechanical behavior (e.g., implemented as a model of how it responds to physical constraints imposed by surrounding tissue and/or actuation commands), for example as described in relation to FIGS. 9A-9D.

At block 1404, in some embodiments, the interatrial septum is crossed by the device delivery sheath. This optionally comprises guided targeting of an interatrial septum crossing location, for example as described in relation to FIGS. 9A-9D, and optionally comprises tests to avoid accidental penetration of the aorta, for example as described in relation to FIG. 9E. Pre-crossing guidance may be provided using images; for example, as shown and/or described in relation to FIG. 13C.

At block 1406, in some embodiments, the device delivery sheath is navigated to a target region of the LAA. Navigation guidance may be provided using images; for example, as shown and/or described in relation to FIG. 13A. A successful navigation result is shown, for example, in FIG. 13A. Navigation of the device optionally comprises use of the LAAO device itself as a position sensing device, for example as described in relation to FIGS. 1A-1F, 2, and/or 3. The position sensing is used, in some embodiments, to monitor and/or guide movements of the device delivery sheath.

At block 1408, in some embodiments, the LAAO device is deployed. Optionally, deployment is monitored and/or guided; for example as described in relation to FIGS. 10A-10D, 11A-11D, and/or 12A-12D. Aspects of FIGS. 4A-4C, 5A-5C, and/or 6A-6C may also be relevant, depending on how the particular LAAO device is configured. Deployment guidance may be provided using images; for example, as shown and/or described in relation to FIG. 13B.

At block 1410, in some embodiments, deployment of the LAAO device is verified. Optionally, verification includes checks described, for example, in relation to FIGS. 10A-10D and/or 11A-11D. Verification guidance may be provided using images; for example, as described in relation to FIG. 13B.

In some embodiments, verification includes one or more of verifying device position, testing its anchoring, confirming its size, and confirming adequate sealing. Anchor testing optionally comprises measuring movement of a deployed LAAO device (for example, as determined from electrical impedance measurements) as a device operator applies a moderate amount of force on it (e.g., tugs on it via an attached catheter). Movement from a starting position (optionally indicated as a starting position by the device operator) may be shown, e.g., as a bar, for example a bar scaled from 0-5 mm. If movement upon exertion of force exceeds a certain threshold (e.g., more than 5 mm, more than 10 mm, more than 20 mm), it indicates a deployment failure, since a properly anchored device should not be moveable more than a relatively small amount.

### Fossa Identification

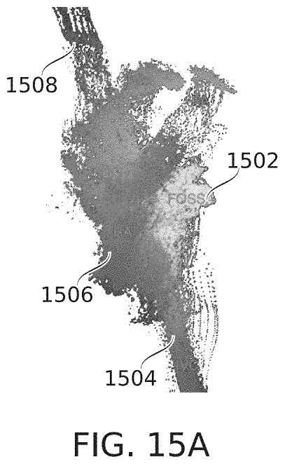

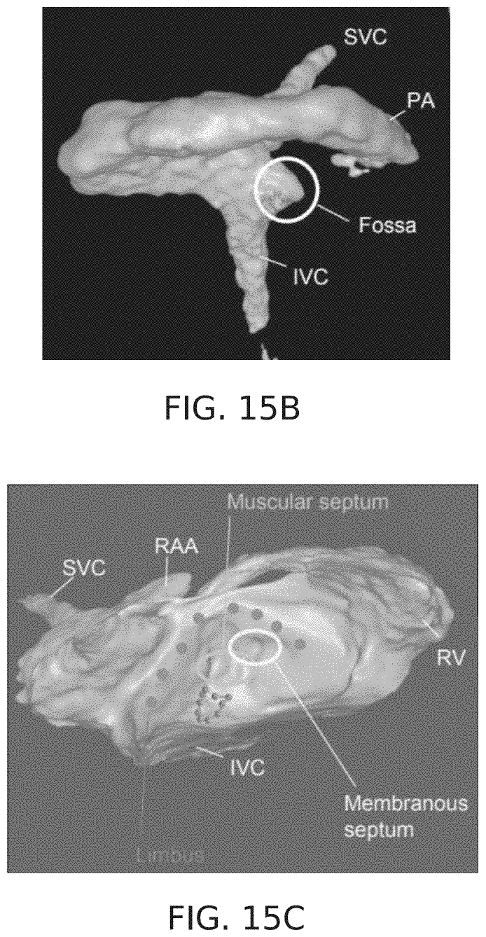

Reference is now made to FIG. 15A, which schematically represents a cloud of position measurements made within a right atrium, from which the position of a fossa 1502 is estimated, according to some embodiments of the present disclosure. Position measurements are made, e.g., using electrodes of an electrode catheter, sensing impedance with respect to a plurality of electrical fields generated to pass through the right atrium.

Indicated in FIG. 15A are position measurements from within the inferior vena cava 1504, from within the superior vena cava 1508, from within the main body of the right atrium 1506 itself, and from within a region near to the fossa ovalis 1502. Insofar as the geometry of the right atrium includes certain characteristic features, measurement positions corresponding to the position of the fossa 1502 can be determined from heuristic features of being, e.g.:
In a medial direction, relative to a point of entry to the right atrium (e.g., from the inferior vena cava).
On a vertical wall in the medial direction.
At most distant positions (relative to a point of entry) in the medial direction along the vertical wall.

Optionally, fossa indications to an operator comprise a graded indication; e.g., color coded; wherein locations are indicated as being “more fossa-like” depending on how distant they are from a point which is most medially distant along the vertical wall in the medial direction. Reference is now made to FIGS. 15B-15C, which schematically represents a reconstructed 3-D view of a right atrium externally (FIG. 15B) and of a cutaway view of a right atrium looking toward the interatrial septal wall (FIG. 15C), according to some embodiments of the present disclosure. In FIG. 15B, positions of the superior vena cava (SVC), pulmonary artery (PA), inferior vena cava (IVC) and fossa ovalis (Fossa) are indicated. The fossa comprises an apex which is a location at which a device such as a catheter and/or device delivery sheath optionally crosses the interatrial septum.

The cutaway view of FIG. 15C focuses on looking at the septal wall comprising the fossa, including within the area of the fossa indications of the muscular septum and the membranous septum. Also shown are the limbus, which is optionally an additional morphological characteristic which helps in the identification of the fossa. Furthermore indicated are portions of the right atrial appendage (RAA) and right ventricle (RA), in addition to the SVC and IVC.

Locating the fossa from measurements made from within the right atrium is a potential advantage for a procedure, in order to assist in establishing relevant targets for crossing, for example, as a constraint on options for crossing considered for methods described in relation to FIGS. 9A-9E, herein.

### Left Atrial Appendage Sizing

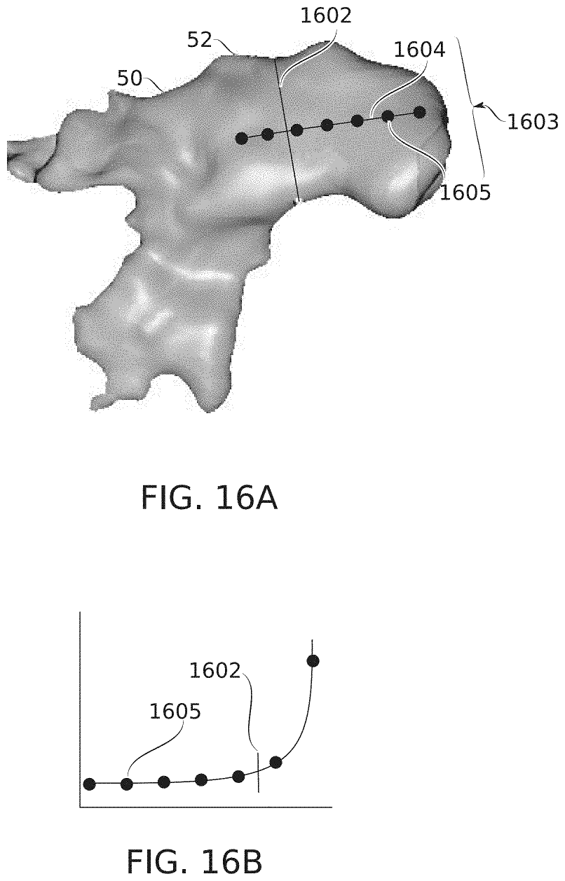

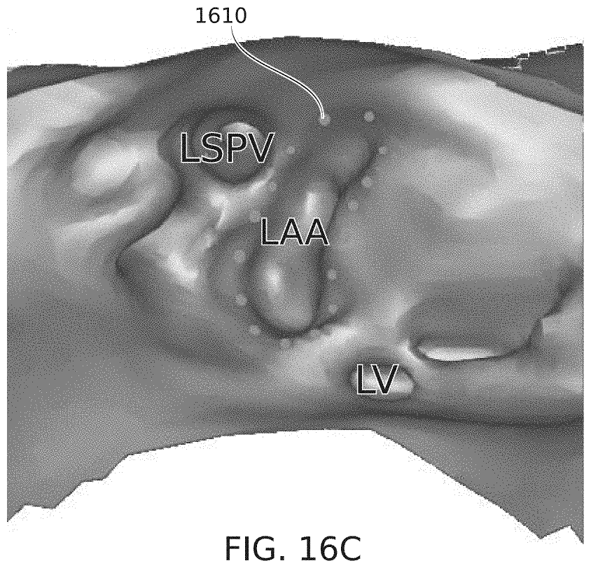

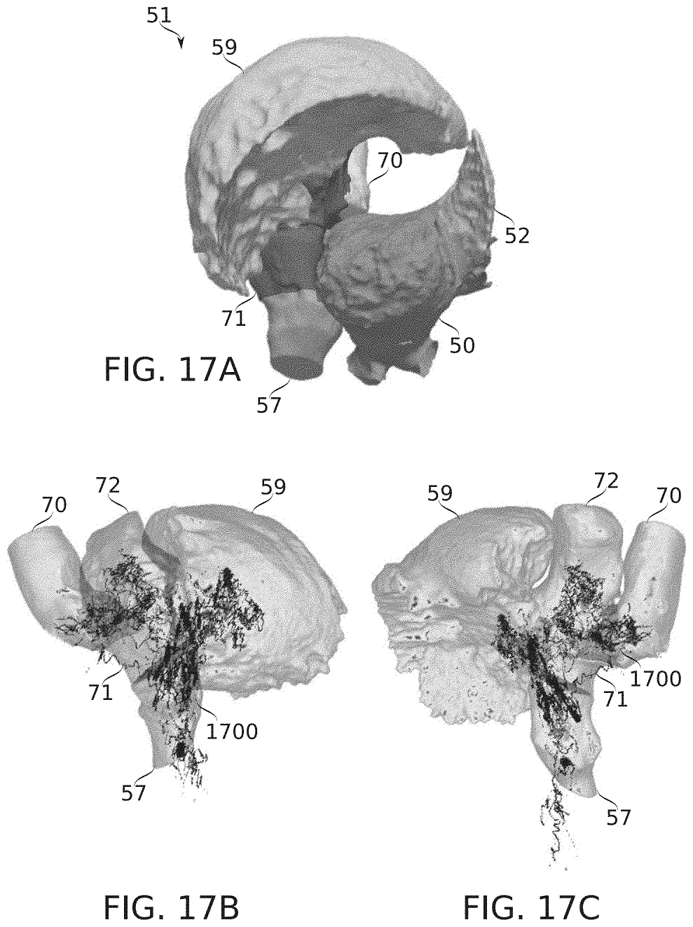

Reference is now made to FIG. 16A, which schematically represents a method of sizing an LAA 52, according to some embodiments of the present disclosure. Reference is also made to FIG. 16B, which schematically represents relative rates of dilution (on the vertical axis) of an impedance and/or dielectric contrast agent as a function of measurement position 1605, according to some embodiments of the present disclosure.

In some embodiments, a targeted position of implantation of an LAAO device positions its back plane at the ostium of the LAA. Optionally, the ostium position of the LAA is found by inserting an injection catheter (e.g., a pigtail catheter) configured with measurement electrodes within the LAA. In FIG. 16A, measurement position dots 1605 represent electrode positions of such a catheter inserted into the LAA 52. Upon injection of a dielectric and/or impedance contrast agent such as saline and/or iodine, the electrodes at positions 1605 register a change in measured values. The change returns to baseline with a certain rate dependent on the rate of dilution at the electrode position 1605, optionally expressed, for example, in terms of % return to baseline per heartbeat. At about the place where the LAA 52 begins to open up into the main lumen of the left atrium 53, the beginning of a relatively rapid rise in dilution rate may be observed (for example, at about position 1602). This position, in some embodiments, is selected as the target back plane position, for example, a backplane as described in relation to FIGS. 4H-4J, herein.

In some embodiments, a width 1603 across LAA 52 is measured corresponding to the width at position 1602. Width 1603 is optionally used to assist in selecting the size of an LAAO device, for example, a device which is at least as big in diameter as width 1603 in unconstrained expansion (and preferably slightly wider, e.g., by at least about 10%).

Reference is now made to FIG. 16A-C, which represents a cutaway view of a portion of the internal lumenal surface of a left atrium, according to some embodiments of the present disclosure. In some embodiments, an ostium into the LAA (dotted line 1610) is markedly non-circular. Optionally, a perimeter length of the ostium at about position 1602 (FIG. 16A) is determined, e.g., a perimeter around dotted line 1610. The width of an LAAO device selected for implantation is adjusted to take into account that the LAAO device, upon expansion, may tend to distort the ostium into a more circular shape, resulting in a reduce maximum width, and an expanded minimum width.

Also shown in FIG. 16C are the ostium of the left superior pulmonary vein (LSPV) and a partial view into the left ventricle (LV).

Registration of CT images and Electrical Position Measurements

Reference is now made to FIG. 17A, which illustrates a segmented CT image of a portion of a heart 51, according to some embodiments of the present disclosure. Reference is also made to FIGS. 17B-17C, which illustrate registration of electrically-measured probe positions represented as a point cloud 1700 (that is, the black dots represent measurements of a measurement cloud) to a segmented CT image of a portion of a heart 51.

Differently shaded regions of FIG. 17A represent different corresponding structures of a heart 51, including right ventricle 59, inferior vena cava 57, superior vena cava 70, right atrium 71, left atrium 50, and left atrial appendage 52. In some embodiments, segmentation and structure identification are carried out automatically; optionally manual segmentation and structure identification are performed.

Semitransparent regions in FIGS. 17B-17C show some of the structures of a heart 51 from different views, including right ventricle 59, inferior vena cava 57, superior vena cava 70, right atrium 71, and right atrial appendage 72. The locations marked in black represent a point cloud 1700 generated from position measurements made using an electrode probe (via measurement of electrical signals) as it visited different portions of the heart structures indicated. As used herein, the term electrical position measurements refers to position measurements made using an electrode probe (via measurement of electrical signals). Measurements of electrical signals may include measurements of current, voltage, and/or impedance by the electrode-carrying probe. While measurements within a right atrium 71 and right atrial appendage 72 are shown, it should be understood that similar registration may be performed for other heart structures; for example, including left atrium 50 and left atrial appendage 52.

Registration of a CT image and/or segmented structures thereof and point cloud 1700 is optionally performed manually. Optionally, automatic registration is performed, for example, by using constraints such as that initial points are measured near an entrance to the heart (e.g., inferior vena cava 57), and/or that the point cloud 1700 should be confined within the bounds of the segmented structures of the CT image. In some embodiments, a centering constraint is used, e.g., a constraint that maximizes wall distance of points in point cloud 1700 while remaining within the segmented structures of the CT image. Examples of systems and methods for registration of intra-body electrical readings (e.g., of probe position) with a pre-acquired three dimensional image are described, for example, in International Patent Publication No. WO2018/078540, the contents of which are included by reference herein in their entirety.

In some embodiments, registration is performed as new data in point cloud 1700 are acquired. By displaying the registered image/point cloud combination, this may be used, for example, to provide CT image-guided navigation of an electrode bearing probe within the heart 51.

Sheath Assembly, Dilator, and Pigtail Catheter

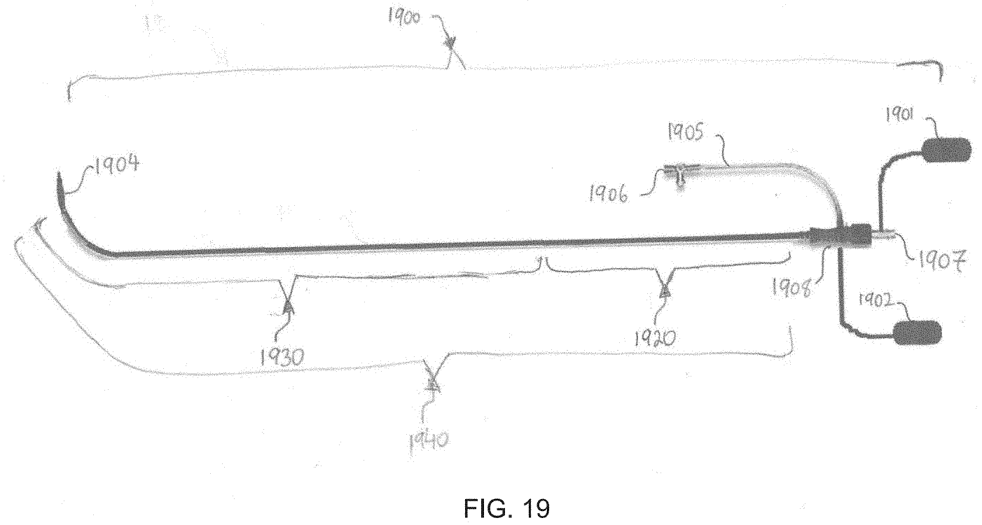

Reference is now made to FIG. 19, which schematically depicts a dilator 1904 inserted into a sheath assembly 1900, according to some embodiments of the present disclosure.

In some embodiments, sheath assembly 1900 and dilator 1904 are configured for performing a trans septal crossing, for example as may be performed to introduce instruments into the left atrium of a heart from a right-atrial access position during a minimally invasive intervention within the left atrium of a heart. More particularly, in some embodiments, sheath assembly 1900 and dilator 1904 are used as part of a procedure for sealing the left atrial appendage (LAA) of the heart.

Functions of sheath assembly 1900 include guiding dilator 1904 to the site of the transseptal crossing, and optionally performing sensing which assists in identifying the site of the transseptal crossing. After septal crossing (e.g., by use of dilator 1904), sheath assembly 1900 may provide a guide for introduction of instruments such as an LAA occluding device into the left atrium. Optionally, sheath assembly 1900 performs sensing within the left atrium which is used to guide the LAA occluding device to its deployment position, and/or to verify LAA occluding device deployment based on leak detection.

In some embodiments, after deployment of an LAA occluding device, sheath assembly 1900 performs sensing within the left atrium which is used to assess the quality of LAA occluding device deployment. Optionally, sheath assembly 1900 is used to introduce contrast agent to the left atrium which assists in assessing the quality of LAA occluding device deployment.

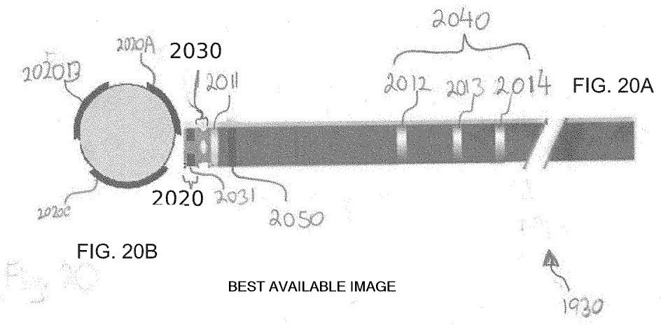

Features of sheath assembly 1900 which support its sensing functions are further described, for example, in relation to FIGS. 20A-20B, herein.

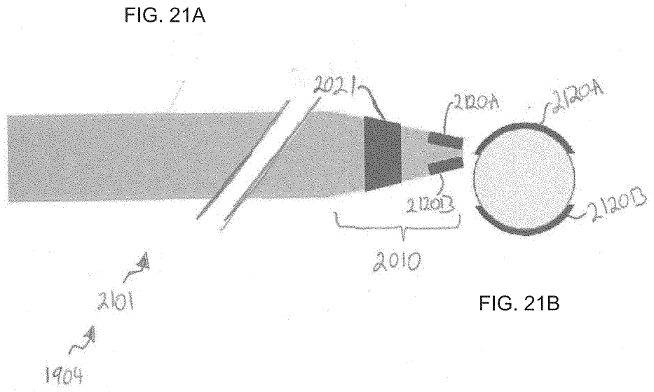

Functions of dilator 1904 include creating and/or widening a septal wall aperture through which instrumentation is to be introduced, and optionally sensing which assists in identifying the site of the transseptal crossing. Details of dilator 1904 supporting these functions are further described, for example, in relation to FIGS. 21A-21B, herein.

In some embodiments, sheath assembly 1900 comprises a tubular sheath body 1940 comprising a distal, more flexible portion 1930, and a proximal, stiffer portion 1920. Stiffening the proximal shaft potion has the potential advantage of enhancing kink resistance and/or pushability of the device. Leaving the distal shaft portion more flexible has potential advantages for steerability, reduction of traumatic risk, and/or maintaining a smaller outer diameter and/or larger inner diameter.

Overall, tubular sheath body 1940 is optionally provided with a working length in a range of about 750 mm to 1000 mm. Outer diameter may be, for example, in a range from about 10 French gauge to about 14 French gauge (about 3.333 mm-4.667 mm; one French gauge unit is equal to ⅓ mm). Inner diameter may be, for example, in a range from about 8.5 French gauge to about 12 French gauge (about 2.833 mm-4 mm).

Proximal stiffer portion 1920, in some embodiments, is about 450 mm long, with the remainder of the working length comprising distal more flexible portion 1930.

Sheath assembly 1900 also comprises haemostasis valve 1908. In some embodiments, sheath assembly 1900 comprises side port 1905 (connected to haemostasis valve 1908), through which fluid materials (e.g., contrast agent) may be optionally introduced into the lumen of tubular sheath body 1904, for example as described in relation to FIGS. 20A-20B. Side port 1905 is optionally terminated by three-way stopcock 1906.

Sheath assembly 1900 comprises connector 1902 which allows making electrical sensing connections to electrodes (e.g., electrodes 2020A-2020C, 2050) positioned upon a distal section of sheath body 1940, for example as further described in relation to FIG. 20A-20B.

In some embodiments, dilator 1904 comprises connector 1901 which allows making electrical sensing connections to electrodes 2111, 2112, 2113 positioned upon a distal section of sheath body 1940, for example as further described in relation to FIGS. 21A-21B. The proximal end of dilator 1904 comprises Luer lock 1907.

Reference is now made to FIGS. 20A-20B, which schematically depict views of a distal portion 1930 of a sheath assembly 1900, according to some embodiments of the present disclosure.

In some embodiments, sheath distal portion 1930 comprises one or more sensing electrodes (e.g., two or more sensing electrodes). In some embodiments, at least two of the electrodes are more particularly configured to sense contact impedance; that is, changes in impedance which are characteristic upon the distal tip making contact with internal tissue (e.g., an interatrial septum). In some embodiments, a plurality of sensing electrodes (e.g., electrodes 2020A, 2020B, 2020C, shown in an axial view in FIG. 20B) are arranged in a pattern extending circumferentially around a section 2020 of sheath distal portion 1930 (e.g., around a periphery of section 2020). The circumferential arrangement potentially provides some resolving power to distinguish the conductive environment in different radial directions. For navigation and/or target finding, this can help to detect, e.g., the side of closest approach to a wall of the body cavity in which the sheath is being navigated—for example, as a cavity wall is approached, the impedance measured by an electrode closest to it goes up faster and/or to a higher level than more distant electrodes. For leak detection, a radial arrangement of electrodes potentially helps to distinguish the radial direction from which leaking contrast fluid is flowing.

In some embodiments, at least one (e.g., one, two, or more) circumferential (band) electrode 2050 is optionally used—additionally or alternatively to sensing electrodes 2020A-2020C—as a sensing electrode in its own right, and/or as a reference electrode for any of sensing electrodes 2020A-2020C. In some embodiments, electrode 2050 is placed proximal to the sensing electrodes, and configured to sense non-contact impedance—that is, impedance which is not sensitive to contact of the distal tip with internal tissue. Used as a reference electrode for sensing electrodes 2020A-2020C, this potentially allows isolating contact impedance changes from other impedance changes which both types of electrodes experience. In some embodiments, electrode 2050 is grounded.

In some embodiments, electrode 2050 is positioned about 6 mm from the distal terminus of distal portion 1930 (or another distance, e.g., at least 5 mm) and is about 1 mm in thickness (along a longitudinal axis of distal portion 1930). Section 2020 is optionally about the same longitudinal thickness (e.g., accommodating electrodes 2020A-2020C sized within a range of about 0.5 mm −2 mm), with the electrodes 2020A-2020C being each, for example, about 1 mm long in the circumferential direction, and/or sized to take up about ⅕ of the total circumference (i.e., 2π/5 radians). Each of the electrodes 2020A-2020C is optionally at a same distance from the distal terminus of distal portion 1930, e.g., a distance between about 0.5 mm and about 2 mm.

In some embodiments, distal portion 1930 comprises one or more radiopaque markers (e.g., 2, 3 or more markers); for example, marker 2011 placed within 6 mm of the distal terminus of distal portion 1930, and/or markers 2040, placed more proximally, e.g., 20 mm, 27 mm, and 33 mm proximal of the distal terminus of distal portion 1930, for radiopaque markers 2012, 2013, and 2014, respectively. In some embodiments radiopaque markers are placed at distances between 15 and 40 mm from the distal terminus of distal portion 1930. Other markers may be placed, for example, at about 24 mm and/or about 30 mm proximal of the distal terminal of distal portion 1930. Distances may be selected to assist in converting a device delivered through the distal portion into a known state of deployment. For example, under X-ray imaging, the alignment of a distal side of the device to a radiopaque marker may indicate whether the device is in a point- or ball-shaped deployment stage. There may be markers provided at different positions, for use with different sized devices.

The thickness of the radiopaque markers may be, for example, about 1 mm along the longitudinal axis of distal portion 1930. Radiopaque markers optionally comprise any (preferably biocompatible) material opaque to X-ray radiation, for example, gold or silver.

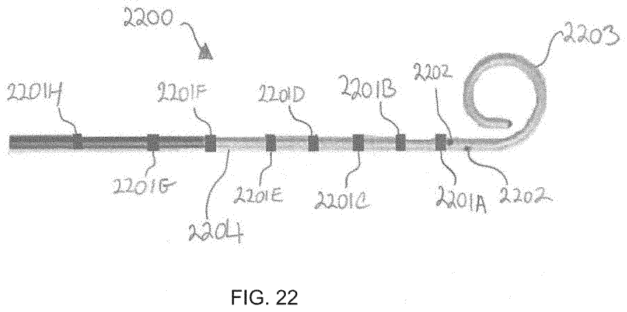

In some embodiments, an optional section 2030 adjacent to the distal terminus of distal portion 1930 comprises a plurality of circumferentially arranged apertures 2031. These apertures 2031 4may be used, for example, to help distribute injected dielectric contrast agent (that is, as irrigation holes). In some embodiments, the irrigation holes are positioned between the more distal and more proximal electrodes; e.g., longitudinally between electrode 2050 and electrodes 2020A, 2020B, and 2020C. Optionally, the sheath distal portion 1930 itself is used for making LAA depth measurements, for example as described in relation to pigtail catheter 2200 of FIG. 22, and then the apertures 2031 allow dielectric contrast fluid to readily escape from the catheter even if the distal end itself is occluded. In some embodiments, sheath distal portion 1930 is provided with additional electrodes spaced along its longitudinal axis, for example as is described in relation to the pigtail catheter 2200.

Optionally, electrodes of sheath 1900 are connected to an apparatus configured to determine a difference between impedance sensed by the distal electrodes 2020A-2020C and impedance sensed by the proximal electrode 2050, and monitor time development of said difference after irrigation of a body part (such as an LAA) in the vicinity of the distal end.

Reference is now made to FIGS. 21A-21B, which schematically depict views of a dilator distal portion 2101 of a dilator 2010, according to some embodiments of the present disclosure.

In some embodiments, dilator distal portion 2101 comprises a tapered dilating portion 2010, which thickens from a distal to proximal direction to help dilate an aperture in a septal wall as it is advanced into it. In some embodiments, the distal side of the taper is about 1 mm in diameter (3 French gauge units), and the proximal side about 3 mm in diameter (e.g., 8-10 French gauge units) There may be provided a first and a second dilator for use in sequence (e.g., extracting the first dilator fully from sheath 1900 before inserting the second to be used). The second dilator has a larger-diameter taper. The first dilator may be particularly adapted (e.g., by the size of an internal lumen of the first dilator) to housing a needle which make an initial puncture by protruding from an end of the dilator. The second dilator may be adapted instead to the delivery of a larger-diameter device, such as a device which is to be delivered across the septal wall (e.g., adapted by having a larger internal diameter than the first dilator). Additionally or alternatively, dividing the dilation task among two dilators optionally allows providing shorter taper lengths for a given taper slope, and a correspondingly reduced distance of penetration of the dilator (which may have an advantage, e.g., for safety).

In some embodiments, dilating portion 2010 is provided with one or more electrodes along its taper; for example (more proximal) band electrode 2021, and/or a plurality of (more distal) circumferentially arranged electrodes 2120A, 2120B. These electrodes may be configured to sense impedance, and to differentially sense contact impedance, as also described for the electrodes of the sheath 1900. Examples of dimensions of these electrodes include about 1 mm thickness along a longitudinal axis of distal portion 2101, and placement with their centers about 0.5 mm proximal to a distal terminus of distal portion 2101 (e.g., for circumferentially arranged electrodes 2120A, 2120B), or about 2.5 mm proximal to that distal terminals (e.g., for band electrode 2021). The circumferentially arranged electrodes 2120A, 2120B may be each sized to extend about ⅓ of the e circumference where they are positioned (i.e., 2π/3 radians). Potential uses of the different electrode arrangements (band vs. spaced circumferential) are as described, e.g., in relation to FIGS. 20A-20B.

Dilator distal portion 2101 is optionally configured to contain and/or receive a needle (e.g., an 18-gauge needle), which can be advanced out of the end of distal portion 2101 to create an initial puncture, e.g., of the interatrial septum.

Optionally, electrodes of dilating portion 2010 are connected to an apparatus configured to determine a difference between impedance sensed by the distal electrodes 2120A, 2120B and impedance sensed by the proximal electrode 2021, and monitor time development of said difference after irrigation of a body part (such as an LAA) in the vicinity of the distal end.

Reference is now made to FIG. 22, which schematically depicts a pigtail catheter 2200, according to some embodiments of the present disclosure.

In some embodiments, pigtail catheter 2200 comprises a curled end 2203, which provides a potential advantage for position stabilizing and/or restricting fluid jetting upon injection of dielectric contrast fluid through pigtail catheter 2200. Curled end 2203 form a coil or loop; for example, about 13 mm high and about 11 mm long when deployed.

Fluid injected into a lumen of pigtail catheter 2200 escape, e.g., through holes 2202 (of which two are indicated; there may be any number of them).

Spaced out along longitudinally along the distal body 2204 of pigtail catheter 2200 are a plurality of electrodes 2201A-2201H. There may be provided, for example, 8 electrodes, each 1 mm in width along a longitudinal axis of pigtail catheter 2204. The respective positioning of these electrodes may be, for example, centered about 12 mm, 16 mm, 20 mm, 24 mm, 28 mm, 35 mm, 40 mm, and 45 mm proximal to a distal side of curled end 2203. Whatever distances are selected for the electrodes may be used to help determine the depth of a closed-ended structure (such as an LAA) to which pigtail catheter 2200 is inserted, in the following fashion:

Curled end 2203 is inserted up to the end of the LAA, until it is prevented from advancing further by contact with the walls of the LAA. A dielectric contrast agent such as saline is then injected via holes 2202, diluting away the fluid (blood) which is normally present. Electrodes near the injection site will sense a change in their electrical environment (e.g., a rise or fall in local impedance) corresponding to the inflow of the dielectric contrast agent. As more fluid is injected, this effect is sensed on more proximally positioned electrodes. However, electrodes which are outside the closed-ended structure will see no or a substantially lessened effect, due to the effect of dilution and/or blood flow preventing a high concentration of the contrast agent from building up around them. Knowing the sensing data, and knowing where the electrodes are, allows calculation to estimate the depth of the structure into which the pigtail catheter has been inserted.

In some embodiments, any two or more of sheath 1900, dilator 1904 and pigtail 220 are provided together as a kit. The dilator 1904 may be longer than the sheath 1900 so that at least 40 mm of it protrudes from sheath 1900 when it is fully inserted thereto. The kit may be provided together with sensing apparatus configured to determine impedance differences between more proximal and more distal electrodes.

### General

It is expected that during the life of a patent maturing from this application many relevant implantable and/or endolumenally operated medical devices will be developed; the scope of the term implantable and/or endolumenally operated medical devices is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and is brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1-45. (canceled)

46. A system for guiding a medical implement comprising an electrically conductive portion inside a body cavity, comprising:
the medical implement;
an electrical field measurement controller;
an electrically conductive structure configured to connect the electrical field measurement controller to an electrically conductive portion of the medical implement, and also to mechanically manipulate the medical implement; and
a processor configured to:
access a rule for transforming electrical field measurements to positions,
receive from said electrical field measurement controller via said conductive structure a set of implement electrical field measurements measured within the body cavity, and
estimate a position of the electrically conductive medical implement using the set of implement electrical field measurements and the rule.

47. (canceled)

48. The system of claim 46, wherein the medical implement comprises an implantable occlusive device.

49. The system of claim 46, wherein the electrically conductive structure comprises a cable, tube, and/or strut operable to extrude a device from an electrically insulating catheter sheath.

50. The system of claim 46, wherein the medical implement comprises an electrically conductive portion having a surface at least 10 mm in one or more dimensions, and is configured to expose the surface of the electrically conductive portion when fully deployed.

51. The system of claim 46, wherein the medical implement comprises a medical implant device configured to attach to and be left in the body.

52. The system of claim 46, wherein the electrical field measurement controller is configured to measure current, voltage, and/or impedance.

53. The system of claim 46, further comprising:
a multi-electrode probe;
electrical field generating electrodes; and
an electrical field generating controller, configured to generate electrical fields using the electrical field generating electrodes.

54. The system of claim 53, wherein the electrical field measurement controller is configured to measure electrical field between a ground electrode and at least two of the electrodes of the multi-electrode probe.

55. The system of claim 54, wherein the processor is further configured to:
receive from the electrical field measurement controller a first set of electrical field measurements measured within the body cavity; and
generate the rule for transforming electrical field measurements to positions based on the first set of electrical field measurements.

56. The system of claim 54, wherein the ground electrode is a body surface electrode.

57. The system of claim 53, wherein the electrical field generating electrodes are body surface electrodes.

58. The system of claim 53, wherein the medical implement comprises an electrically conductive portion at least twice as big as each of the measuring electrodes of the multi-electrode probe in one or more dimensions, and is configured to expose the electrically conductive portion when fully deployed.

59. The system of claim 53, wherein the first set of electrical measurements comprises measurements of one or more electrical fields extending through the body cavity, measured using electrodes of the multi-electrode probe.

60. The system of claim 59, wherein the rule for transforming electrical field measurements to positions transforms electrical field readings of the first set of electrical field measurements to positions of electrodes of the multi-electrode probe.

61. The system of claim 53, wherein the processor is configured to generate the rule using inter-electrode distances of the multi-electrode probe.

62. A method of guiding an electrically conductive medical implement inside a body cavity, the method comprising:
moving the electrically conductive medical implement to at least partially extrude it from an insulating sheath, thereby at least partially expanding a shape of the electrically conductive medical implement;
receiving indications of measurements of a plurality of electrical fields measured using the conductive medical implement as a measuring electrode; and
adjusting the moving, based on the received indications;
wherein the moving is performed by exerting force through mechanical manipulation of a conductive structure mechanically interconnected with the conductive medical implement, and the measurements are of electrical signals transmitted from the conductive medical implement and along the conductive structure.

63. The method of claim 62, wherein the moving and measuring are performed simultaneously.

64. The method of claim 62, wherein the conductive medical implement comprises an implantable occlusive device.

65. The method of claim 62, wherein the conductive medical implement comprises an electrically conductive portion having a surface at least 10 mm in one or more dimensions.

66. The method of claim 62, wherein the electrically conductive portion is exposed by the moving upon being at least partially extruded.
